(19) United States
(12) Patent Application Publication
(10) Pub. No.: US 2008/0044484 A1
(43) Pub. Date: Feb. 21, 2008

(54) Use of polymeric nanoparticles for vaccine delivery

(75) Inventor: Boris R. Minev, San Diego, CA (US)

Correspondence Address: DLA PIPER US LLP, 4365 EXECUTIVE DRIVE, SUITE 1100, SAN DIEGO, CA, 92121-2133, US

(73) Assignee: THE REGENTS OF THE UNIVERSITY OF CALIFORNIA, Oakland, CA (US)

(21) Appl. No.: 11/820,753

(22) Filed: Jun. 19, 2007

Related U.S. Application Data
(63) Continuation-in-part of application No. 11/631,557.
(60) Provisional application No. 60/586,847, filed on Jul. 8, 2004.
(60) Provisional application No. 60/586,900, filed on Jul. 8, 2004.
(60) Provisional application No. 60/586,997, filed on Jul. 8, 2004.
(60) Provisional application No. 60/586,914, filed on Jul. 8, 2004.

(51) Int. Cl. C07K 14/00; A61K 38/02; A61K 38/08; A61K 38/10; C07K 2/00; C07K 7/00; A61K 38/16; A61K 9/14; A61P 35/00

(52) U.S. Cl. 424/499; 514/12; 514/13; 514/15; 514/2; 530/300; 530/324; 530/326; 530/328

(57) ABSTRACT

The invention relates generally to the treatment and prevention of human cancer and viral diseases. More specifically, this invention relates to development of a new generation of vaccines that rely on eliciting cellular immune responses, specifically induction of cytotoxic T lymphocytes (CTL), against cancer cells and virus-infected cells via administration of a polymeric nanoparticle containing a vaccine comprising a fusion peptide or a modified peptide. Such a fusion peptide is composed of an insertion signal sequence and a peptide derived from a tumor antigen or a viral antigen, which improves antigen presentation and induces CTL with higher efficiency against cancer cells and virus-infected cells. An exemplary peptide utilized in the invention is Mart-1:27-35 peptide.

### CROSS REFERENCE TO RELATED APPLICATION(S)

This application is a continuation-in-part of U.S. Ser. No. 11/631,557, filed on Jan. 3, 2007, which is a 35 U.S.C. §371 National Stage application of PCT Application No. PCT/US2005/024216 filed Jul. 8, 2005, which claims the benefit under 35 U.S.C. §119(e) to U.S. Application Ser. No. 60/586,847, filed Jul. 8, 2004, now abandoned, to U.S. Application Ser. No. 60/586,900 filed Jul. 8, 2004, now abandoned, to U.S. Application Ser. No. 60/586,997 filed Jul. 8, 2004, now abandoned, and to U.S. Application Ser. No. 60/586,914 filed Jul. 8, 2004, now abandoned; and claims the benefit of priority under 35 U.S.C. §119(e) of U.S. Ser. No. 60/815,410, filed Jun. 20, 2006. The disclosure of each of the prior applications is considered part of and is incorporated by reference in the disclosure of this application.

### GOVERNMENTAL INTERESTS

This invention was made in part with government support under Grant No. W81XWH-04-1-0863 awarded by the U.S. Army Medical Research and Material Command. The government has certain rights in this invention.

### FIELD OF THE INVENTION

The present invention relates generally to the treatment and prevention of human cancer or viral disease and, more specifically, to use of nanoparticles as antigen delivery vehicles for synthetic vaccines against pathogens or cancers.

### BACKGROUND INFORMATION

Cytotoxic T lymphocytes (CTL) appear to be among the most direct and effective elements of the immune system that are capable of generating anti-tumor immune responses. Tumor cells expressing the appropriate tumor-associated antigens can be effectively recognized and destroyed by these immune effector cells, which may result in dramatic clinical responses. Both the adoptive transfer of tumor-reactive CTL and active immunization designed to elicit CTL responses have been reported to lead to significant therapeutic anti-tumor responses in patients with cancer.

In recent years, peptides derived from tumor-associated antigens (TAA) have been identified for a variety of human cancers. Thus far, however, effective peptide vaccination of patients with cancer has been limited to very few trials. A major obstacle for effective immunotherapy of cancer is that most TAA described to date are expressed in one or a few tumor types, and among patients with these types of tumors, TAA expression is not universal. Tumor cells express a variety of antigens on their surface, in context with the MHC molecules. In many cases however, tumor cells cannot induce an effective T cell response through naive T cells, partly because they lack the necessary co-stimulatory molecules. The relative paucity of responsiveness after conventional peptide vaccination may also be due to the fact that the peptides do not efficiently enter the antigen-presenting cells and do not translocate through the endoplasmic reticulum membrane in order to associate with the MHC molecules. In addition, the immunogenic tumor peptides have short half-life in vivo, and their affinity to class I MHC molecules is not optimal.

Significant advances in biotechnology and biochemistry have led to the discovery of a large number of cancer vaccines based on peptides and proteins. However, the development of suitable and efficient carrier systems remains a major challenge since the vaccine bioavailability is limited by enzymatic degradation. Polymeric nanoparticles, defined as solid particles with a size in the range of 10-1000 nm, may allow encapsulation of the vaccines inside a polymeric matrix, protecting them against enzymatic and hydrolytic degradation. In addition, the nanoparticle vaccine approach offers the possibility of providing tailor-made properties of the vaccine materials that may improve their function—variables include particle size, surface charge, and hydrophobicity.

A widespread barrier for the activity of peptides that function intracellularly is cytoplasmic delivery. The biomolecules usually enter cells through the process of fluid-phase or receptor-mediated endocytosis and are initially localized in the endosomal compartment. A high percentage of these biomolecules are subsequently trafficked to lysosomes, which results in high levels of protein degradation, limiting antigen delivery. Thus, there is a significant need to design and synthesize carriers that can enhance the intracellular delivery of biotherapeutics, in particular to overcome the important barrier of lysosomal trafficking. The issue of cytoplasmic delivery is particularly important for vaccine development, where antigenic peptides must reach the cytoplasm of antigen-presenting cells (APC) to enter the MHC class I pathway for subsequent stimulation of CD8+ cytotoxic T lymphocytes (CTL). The use of endosomal releasing proteins and peptides in gene and protein delivery systems has been widely investigated, but potential limitations of cost, stability, and immunogenicity make alternative synthetic carrier systems highly desirable.

Dendritic cells (DCs) are the most potent professional antigen presenting cells (APCs), and help trigger T-cell mediated immune response. Therefore, immunotherapy utilizing DCs has become a promising therapeutic modality in recent years. Therefore, there remains a need in the art to enhance and prolong the antigen presentation by human DCs by using nanoparticles in cancer immunotherapy.

### SUMMARY OF THE INVENTION

The invention relates generally to the treatment and prevention of human cancer and viral diseases. More specifically, the invention relates to development of a new generation of peptides and peptide vaccines delivered via polymeric nanoparticles for cancer and viral diseases that rely on eliciting cellular immune responses against cancer cells and virus-infected cells.

In one aspect, the invention provides nanoparticles containing peptides that induce the activity of CTL against cancer cells. In one embodiment, the invention provides nanoparticles containing peptides and peptide vaccines derived from MART-1. In another embodiment, the invention provides nanoparticles containing peptides and peptide vaccines derived from OFA/iLRP. In yet another embodiment, the invention provides nanoparticles containing peptides and peptide vaccines derived from STEAP. In yet another embodiment, the invention provides nanoparticles containing non-HLA-A2 peptides and peptide vaccines derived from PRAME.

The peptides of the invention may be modified using any of the approaches described in the invention. In one embodiment, the peptides are operably linked to a signal sequence.

In another aspect, the invention provides a method of treating or preventing cancer by administering a nanoparticle containing a class I restricted peptide. The cancer may be any type of cancer expressing the antigens PRAME, OFA/iLRP, STEAP, SURVIVIN, or MART-1. In one embodiment, the cancer is lung cancer. In another embodiment, the cancer is breast cancer. In yet another embodiment, the cancer is prostate cancer. In yet another embodiment, the cancer is a brain tumor.

In another aspect, the invention provides a nanoparticle containing one or more fusion peptides for treating or preventing cancer or virus-infected cells. Such fusion peptides are composed of an insertion signal sequence and an antigen-derived peptide, which improves antigen presentation and/or induces antitumor and antiviral CTL with higher efficiency. The vaccines of the invention are useful for treating or preventing cancer or virus-infected cells as described herein.

In certain embodiments, the invention nanoparticles may further be administered in combination with a therapeutic agent. Exemplary therapeutic agents include, but are not limited to anti-inflammatory agents, antimicrobial agents, antihistamines, chemotherapeutic agents, antiangiogenic agents, immunomodulators, therapeutic antibodies or protein kinase inhibitors, e.g., a tyrosine kinase inhibitor. Other therapeutic agents that may be administered in combination with invention nanoparticles include protein therapeutic agents such as cytokines, immunomodulatory agents, anticancer agents and antibodies.

In another aspect, the invention provides kits comprising the compositions of the invention. In one embodiment, the kit further provides instructions for practicing the methods of the invention.

### BRIEF DESCRIPTION OF THE DRAWINGS

FIG. 1 sets forth the amino acid sequence of the full length PRAME protein (SEQ ID NO: 1).

FIG. 2 sets forth the nucleic acid (SEQ ID NO: 78) and amino acid sequence of the full length OFA/iLRP protein (SEQ ID NO: 70).

FIG. 3 sets forth the amino acid sequence of the full length STEAP protein (SEQ ID NO: 95).

FIG. 4 shows the results of loading/pulsing T2 cells with peptide constructs composed of synthetic signal sequences attached to amino-terminus or to carboxy-terminus of HER2/neu₄₈₋₅₆. T2 cells were loaded (left column) or pulsed (right column) with ES-HER₄₈₋₅₆ (♦), HER₄₈₋₅₆-ES (⋄), IS-HER₄₈₋₅₆ (▾), HER₄₈₋₅₆-IS (∇) or HER₄₈₋₅₆ (▪). At different periods after loading, T2 cells were used as targets in ⁵¹Cr-release assays for the CTL.

FIG. 5 shows the results of loading/pulsing T2 cells with peptide constructs composed of synthetic signal sequences attached to amino-terminus or to carboxy-terminus of HER2/neu₃₆₉₋₃₇₇. T2 cells were loaded (left column) or pulsed (right column) with ES-HER₃₆₉₋₃₇₇ (♦), HER₃₆₉₋₃₇₇-ES (⋄), IS-HER₃₆₉₋₃₇₇ (▾), HER₃₆₉₋₃₇₇-IS (∇) or HER₃₆₉₋₃₇₇ (▪). At different periods after loading, T2 cells were used as targets in ⁵¹Cr-release assays for the CTL.

FIG. 6 shows the results of loading/pulsing T2 cells with peptide constructs composed of synthetic signal sequences attached to amino-terminus or to carboxy-terminus of HER2/neu₆₅₄₋₆₆₂. T2 cells were loaded (left column) or pulsed (right column) with ES-HER₆₅₄₋₆₆₂ (♦), HER₆₅₄₋₆₆₂-ES (⋄), IS-HER₆₅₄₋₆₆₂ (▾), HER₆₅₄₋₆₆₂-IS (∇) or HER₆₅₄₋₆₆₂. At different periods after loading, T2 cells were used as targets in ⁵¹Cr-release assays for the CTL.

FIG. 7 shows the results of loading/pulsing T2 cells with peptide constructs composed of synthetic signal sequences attached to amino-terminus or to carboxy-terminus of HER2/neu₇₈₉₋₇₉₇. T2 cells were loaded (left column) or pulsed (right column) with ES-HER₇₈₉₋₇₉₇ (♦), HER₇₈₉₋₇₉₇-ES (⋄), IS-HER₇₈₉₋₇₉₇ (▾), HER₇₈₉₋₇₉₇-IS (∇) or HER₇₈₉₋₇₉₇ (▪). At different periods after loading, T2 cells were used as targets in ⁵¹Cr-release assays for the CTL.

FIG. 8 shows the results of loading/pulsing T2 cells with peptide constructs composed of HER2/neu₄₈₋₅₆ incorporated into synthetic signal sequences. T2 cells were loaded (left column) or pulsed (right column) with HE₄₈₋₅₆-IN-AF (●), HER₄₈₋₅₆-IN-ES (▴) or HER₄₈₋₅₆ (▪). At different periods after loading, T2 cells were used as targets in ⁵¹Cr-release assays for the CTL.

FIG. 9 shows the results of loading/pulsing T2 cells with peptide constructs composed of HER2/neu₃₆₉₋₃₇₇ incorporated into synthetic signal sequences. T2 cells were loaded (left column) or pulsed (right column) with HER₃₆₉₋₃₇₇-IN-AF (●), HER₃₆₉₋₃₇₇-IN-ES (▴) or HER₃₆₉₋₃₇₇ (▪). At different periods after loading, T2 cells were used as targets in ⁵¹Cr-release assays for the CTL.

FIG. 10 shows the results of loading/pulsing T2 cells with peptide constructs composed of HER2/neu₆₅₄₋₆₆₂ incorporated into synthetic signal sequences. T2 cells were loaded (left column) or pulsed (right column) with HER₆₅₄₋₆₆₂-IN-AF (●), HER₆₅₄₋₆₆₂-IN-ES (▴) or HER₆₅₄₋₆₆₂ (▪). At different periods after loading, T2 cells were used as targets in ⁵¹Cr-release or the CTL.

FIG. 11 shows the results of loading/pulsing T2 cells with peptide constructs composed of HER2/neu₇₈₉₋₇₉₇ incorporated into synthetic signal sequences. T2 cells were loaded (left column) or pulsed (right column) with HER₇₈₉₋₇₉₇-IN-AF (●), HER₇₈₉₋₇₉₇-IN-ES (▴) or HER₇₈₉₋₇₉₇ (▪). At different periods after loading, T2 cells were used as targets in ⁵¹Cr-release or the CTL.

FIG. 12 shows the results of loading/pulsing breast cancer cells MDA-MB-231 with peptide constructs composed of synthetic signal sequences attached to amino-terminus or to carboxy-terminus of HER2/neu₃₆₉₋₃₇₇. MDA-MB-231 cells were loaded (left column) or pulsed (right-column) with ES-HER/neu₃₆₉₋₃₇₇ (♦), HER2/neu₃₆₉₋₃₇₇-ES (⋄), IS-HER2/neu₃₆₉₋₃₇₇ (▾), HER2/neu₃₆₉₋₃₇₇-IS (∇) or HER2/neu₃₆₉₋₃₇₇ (▪). At different periods after loading, MDA-MB-231 cells were used as targets in ⁵¹Cr-release assays for the CTL.

FIG. 13 shows the results of loading/pulsing breast cancer cells MDA-MB-231 with peptide constructs composed of synthetic signal sequences attached to amino-terminus or to carboxy-terminus of HER2/neu₆₅₄₋₆₆₂. MDA-MB-231 cells were loaded (left column) or pulsed (right-column) with ES-HER2/neu₆₅₄₋₆₆₂ (♦), HER2/neu₆₅₄₋₆₆₂-ES (⋄), IS-HER2/neu₆₅₄₋₆₆₂ (▾), HER2/neu₆₅₄₋₆₆₂-IS (∇) or HER2/neu₆₅₄₋₆₆₂ (▪). At different periods after loading, MDA-MB-231 cells were used as targets in ⁵¹Cr-release assays for the CTL.

FIG. 14 shows the results of loading/pulsing breast cancer cells MDA-MB-231 with peptide constructs composed of HER2/neu₃₆₉₋₃₇₇ incorporated into synthetic signal sequences. MDA-MB-231 cells were loaded (left column) or pulsed (right column) with HER₃₆₉₋₃₇₇-IN-AF (●), HER₃₆₉₋₃₇₇-IN-ES (▴) or HER₃₆₉₋₃₇₇ (▪). At different periods after loading, MDA-MB-231 cells were used as targets in ⁵¹Cr-release assays for the CTL.

FIG. 15 shows the results of loading/pulsing breast cancer cells MDA-MB-231 with peptide constructs composed of HER2/neu₆₅₄₋₆₆₂ incorporated into synthetic signal sequences. MDA-MB-231 cells were loaded (left column) or pulsed (right column) with HER₆₅₄₋₆₆₂-IN-AF (●), HER₆₅₄₋₆₆₂-IN-ES (▴) or HER₆₅₄₋₆₆₂ (▪). At different periods after loading, MDA-MB-231 cells were used as targets in ⁵¹Cr-release assays for the CTL

FIG. 16 is a graph showing loading of dendritic cells with HER2/neu-derived peptides fused to synthetic signal sequences.

FIG. 17 is a graph showing loading of dendritic cells with HER2/neu-derived peptides included within synthetic signal sequences.

FIG. 18 illustrates the path of transport of peptides into a cell and expression of the peptide on the cell surface.

FIG. 19 sets forth the amino acid sequence of the full length SURVIVIN protein (SEQ ID NO: 193).

FIG. 20 is a schematic diagram showing the generation of human DCs.

FIG. 21 is a graphical diagram showing the flow cytometric phenotype of human imDCs and mDCs generated in this study. The imDCs were collected on day 7 before adding LPS, and mDCs were harvested on day 9. Washed with PBS and stained with cross-reactive anti-human monoclonal antibodies against FITC or PE labeled anti-human HLA-DR, CD80, CD83 and CD86. Meanwhile, FITC-mouse IgG1, PE-mouse IgG1, and PE-mouse IgG2a were used for the isotype controls. Gates were set to exclude debris and nonviable cells. The imDCs have low amounts of MHC II, CD86 and CD83, and the mDCs show an up-regulation of MHC II, CD86 and CD83.

FIG. 22 is a pictorial diagram showing scanning electron microscopy of the nanoparticles. Bar represents 1000 nm. Magnification 60,000×.

FIG. 23 is a pictorial diagram showing internalization of PLGA nanoparticles containing Mart-1: 27-35 peptide in human imDCs. The imDCs were analyzed after 1 h incubation with nanoparticles containing Mart-1:27-35/coumarin 6 (A), and coumarin 6 only without any nanoparticles (B) then washed briefly with PBS, the cells were fixed in 1% paraformaldehyde. These slides were visualized under a fluorescence microscope.

FIG. 24 is a graphical diagram showing FACS analysis of human imDCs incubated with nanoparticles containing fluorescein (coumarin 6). The FACS analysis was performed on a FACScan and using the Cell Quest software (BD Bioscience, San Jose, Calif.) for data analysis. Human imDCs on day 7 were harvested and incubated with nanoparticles containing Mart-1/coumarin 6 for 1 h at 37° C., then washed, and resuspended in PBS containing 0.5% BSA. Gates were set to exclude debris and nonviable cells. The number within the histogram plot (100%) represents the percentage of nanoparticles-fluorescence human DCs based on the whole human DCs population.

FIGS. 25A and 25B are graphical diagrams showing phenotypic analysis of human DCs with and without PLGA nanoparticles. Human imDCs generated from HLA-A2 positive healthy donors were loaded with nanoparticles (100 μg/ml) containing Mart 1: 27-35 peptide in the absence (for imDCs) or presence (for mDCs) of LPS 100 ng/ml. The human DCs on day 7 were harvested and divided into the following two groups: Non-NPs group and NPs group. The former group containing two subgroups (1). imDCs; (2). imDCs+LPS-48 h; the later group also containing two groups (3). imDCs+NPs-1h; (4). imDCs+NPs-48 h. Samples in group (1), those are imDCs only on day 7; imDCs treated with LPS for 48 h were included in group (2); the imDCs incubated with NPs for 1 h and 48 h, respectively were included in group 3 and 4, All the DCs samples at different time points were collected for testing the surface markers of HLA-DR, CD80, CD83, and CD86 on the human DCs. The pregated DCs containing NPs (solid area) while the open plots represent the human imDCs stained with isotype controls (FITC-IgG1, PE-IgG1, and PE-IgG2a).

FIG. 26 is a graphical diagram showing 7 PLGA nanoparticles prolonged Mart-1:27-35 presentation by human DCs. Human DCs were incubated with Mart-1:27-35 (0.5 μg/ml), PLGA nanoparticles containing Mart-1:27-35, or nanoparticles without any peptide (control nanoparticles, CNP), and then cocultured with TIL1235 cells at a ratio of 1:1 for ELISpot assay. Data shows mean±SD (n=3).

FIG. 27 sets forth the amino acid sequence of the full length MART-1 protein (SEQ ID NO: 195).

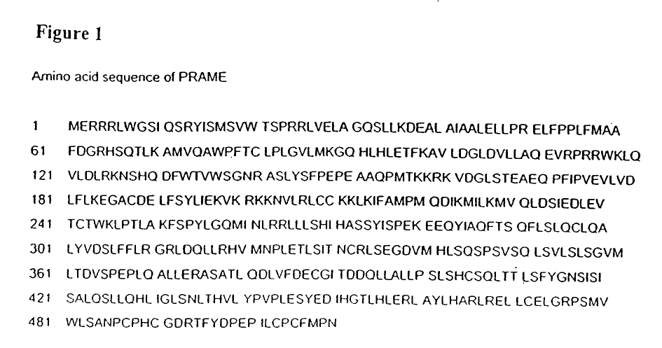

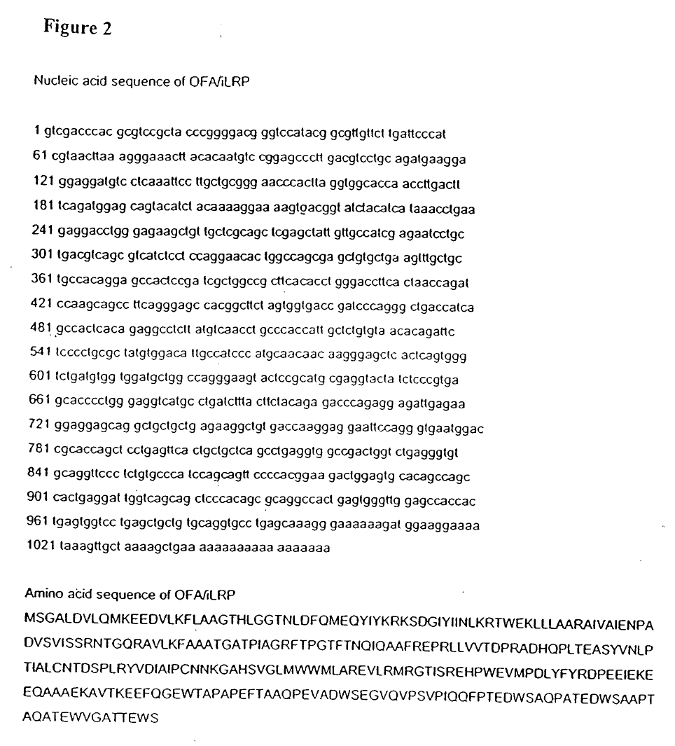

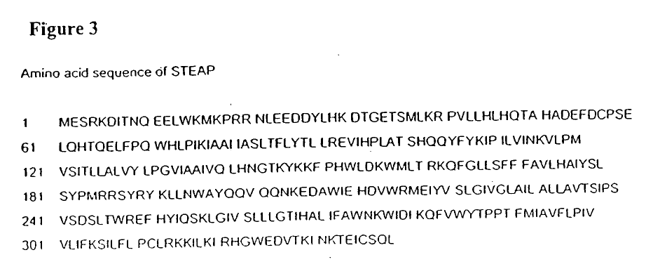

### DETAILED DESCRIPTION OF THE INVENTION

The present invention relates generally to the treatment and prevention of human cancer and viral diseases and, more specifically, to use of nanoparticles to enhance and prolong the antigen presentation by human DCs as a means to develop a new generation of vaccines and vaccine delivery systems for cancer and viral diseases that rely on eliciting cellular immune responses.

Dendritic cells (DCs) represent a class of professional antigen-presenting cells, and have been used for cancer immunotherapy in clinical trails. Peptides derived from tumor antigens can be loaded onto human DCs. Unfortunately, this kind of binding complex of MHC class I/peptide, can only last a few hours, which has been a major problem in vaccine efficacy. Accordingly, in one embodiment, Poly (D,L-lactide-co-glycolide) (PLGA) nanoparticles have been engineered containing MHC class I-restricted Mart-1:27-35 peptide (SEQ ID NO: 194) using emulsion solvent evaporation techniques. As used herein, “Mart-1:27-35” refers to a peptide consisting of amino acid residues 27-35 of the full length Mart-1 protein (SEQ ID NO: 195). PLGA has generated immense interest because of its favorable properties, which include good biocompatibility, biodegradability, and mechanical strength. In addition, PLGA is easy to formulate into different devices for carrying a variety of drug classes such as DNA, peptides, proteins, and micromolecules.

Immature human DCs take up PLGA nanoparticles very easily and present antigens from encapsulated peptides. Thus, in one embodiment, PLGA nanoparticles containing Mart-1:27-35 peptide (SEQ ID NO: 194) are used for enhancing and prolonging antigen presentation by human DCs upon phagocytosis of nanoparticles, when compared with human DCs loaded with soluble Mart-1 peptide. These data may be of high significance for cancer immunotherapy because nanoparticles can markedly enhance and prolong the antigen presentation by human DCs, which is very important for the success of the immunotherapeutic approaches.

Antitumor and antiviral cytotoxic T lymphocytes (CTL) can recognize and kill cancer cells and virus-infected cells, but only if they recognize complexes of peptides associated with the major histocompatibility complex (MHC) class I molecules on the cell surface. CTL appear to be among the most direct and effective elements of the immune system that are capable of generating anti-tumor immune responses. Tumor cells expressing the appropriate tumor-associated antigens can be effectively recognized and destroyed by these immune effector cells, which may result in dramatic clinical responses in a limited number of patients. The paucity of responsiveness in most patients may be due to the inefficient presentation of the antigens used to immunize patients with cancer. Consequently, methods to overcome this obstacle should lead to a marked improvement in antigen presentation and induction of potent anti-tumor CTL.

The recognition of antigens by specific CTL is essential for a successful anti-cancer response. CTL recognize peptides generated from intracellular proteins that are presented by MHC class I molecules on the cell surface. In the cytosol, intracellular proteins are degraded to peptide fragments by multicatalytic protease complexes, the proteasomes. For binding and stabilization of MHC class I molecules these peptides are translocated across the membrane of the endoplasmic reticulum (ER) by the TAP peptide transporter in an ATP-dependent fashion. Following biosynthesis into the ER membrane, MHC class I molecules transiently associate with various helper molecules (chaperones) that facilitate folding and peptide loading. After successful peptide loading MHC class I molecules leave the ER to the cell surface. Once on the cell surface, the peptide is recognized by CTL, which can then kill the cancer cell or virus-infected cell. The present invention, therefore, provides novel peptides, which induce CTL against the cancer or virus on the surface of which the peptides are present, for treatment and prevention of human cancer and virus-infected cells.

One of the most promising of these new antigens, PRAME, is a member of the cancer/testis family of antigens. PRAME is a particularly attractive antigen because it is widely expressed in many different tumor types, but not in normal tissues, except testis. This antigen is detectable in many lung cancers, as well as in melanoma, renal cell cancer, breast cancer, acute leukemias, and multiple myeloma. Undesirable autoimmune reactivity against the few tissues expressing PRAME at low levels is not to be expected, because expression levels are too low to ensure CTL recognition, as shown in vitro with human MAGE-specific CTL and in vivo in a murine p53 model. The high immunogenicity of PRAME, and its broad tumor expression make this protein a very promising target for tumor-specific vaccination strategies.

Accordingly, in one embodiment, the invention provides nanoparticles containing PRAME-derived peptides for inducing CTL against cancer or virus-infected cells. By “PRAME-derived sequence” is meant an amino acid sequence with: (i) terminal modifications to inhibit proteolytic degradation of the PRAME peptides; (ii) amino-acid substitutions at HLA-A2.1 binding anchor positions to enhance MHC Class I binding affinity of the PRAME peptides; (iii) amino acid substitutions at NON-anchor positions to enhance the T cell receptor binding affinity for the peptide-MHC complex, or (iv) insertion signal sequences to enhance the immunogenicity of the PRAME peptides.

Four peptides within the PRAME protein sequence have been utilized in the invention to design optimized synthetic vaccines (Table 1).

TABLE 1

HLA-A2.1-restricted peptides,
identified within the PRAME sequence

| | | |
|---|---|---|
| PEPTIDES | SEQUENCE | REFERENCE |

| | | |
|---|---|---|
| PRAME₁₀₀₋₁₀₈ | VLDGLDVLL | Kessler, J. (J.Exp.Med. 193:73-88, 2001) |
| | (SEQ ID NO: 2) | |
| PRAME₁₄₂₋₁₅₁ | SLYSFPEPEA | Kessler, J. (J.Exp.Med. 193:73-88, 2001) |
| | (SEQ ID NO: 3) | |
| PRAME₃₀₀₋₃₀₉ | ALYVDSLFFL | Kessler, J. (J.Exp.Med. 193:73-88, 2001) |
| | (SEQ ID NO: 4) | |
| PRAME₄₂₅₋₄₃₃ | SLLQHLIGL | Kessler, J. (J.Exp.Med. 193:73-88, 2001) |
| | (SEQ ID NO: 5) | |

Another promising target is the oncofetal antigen (OFA/iLRP) identified as a 37-44 kDa immunogenic glycoprotein expressed in all human tumors examined and also in embryos/early fetuses, but not in term fetus, neonate or adult normal tissues. It was found that OFA reappears as an immunogen in early tumor development, which gives all tumor cells the capacity to activate OFA-specific CTL. Recently, it was found that an oncofetal antigen (OFA/iLRP) could induce in vitro OFA/iLRP-specific effector and regulatory T lymphocytes in patients with cancer. OFA/iLRP is expressed during early to mid-gestation fetal development and re-expressed as a surface antigen by tumor cells soon after transformation. The antigen is detectable on all types of human and rodent tumors tested, but cannot be detected on normal cells.

In one embodiment, the invention provides the identification of class I-restricted peptides derived from the widely expressed tumor antigen OFA/iLRP. These natural and modified peptides might be used directly to immunize patients with cancer. Dendritic cells loaded with the OFA/iLRP peptides can also be used to elicit powerful anti-tumor immune responses. In addition, the OFA/iLRP-specific CTL might be extremely useful for cellular immunotherapy of cancer.

Dendritic cells (DCs) are the most potent antigen-presenting cells (APCs) and play a critical role in initiating primary T cell responses (Banchereau, 1998 #1). DCs not only play important roles in regulating immune responses in cancer, but also have the ability to initiate and maintain primary immune responses. All endocytic cells, including DCs, macrophages and in some circumstances B cells, can present low levels of exogenous antigens. They efficiently target internalized proteins to the MHC class I presentation pathway, making antigen internalization a prerequisite for cross-presentation (Shen, 2006 #9). The immature DCs are characterized by high endocytic activity and low T-cell activation potential. DCs display an extraordinary capacity to stimulate naive T cells and initiate the primary immune response through the activation of lymphocytes (Banchereau, 2004 #4; Banchereau, 1998 #37; Banchereau, 1998 #8).

Once exposed to foreign pathogens, the immature DCs are rapidly activated and become mature DCs, which show strong expression of HLA-DR, and costimulatory molecules (CD40, CD80, CD86) as well as a specific maturation marker, CD83 (Cella, 1997 #43; Sallusto, 1994 #51; Zhou, 1996 #1). Pilot clinical trials indicated that DCs loaded with tumor antigens or whole tumor cell derivatives could induce tumor-specific immune responses in various cancers including B-cell lymphoma, melanoma and prostate cancer (Sauer, 2005 #1; Sauer, 2005 #1; Rosenblatt, 2005 #31). Owing to the very low number of DCs in the blood circulation, a variety of sources have been used to generate DCs including monocytes, CD34+ stem cells and even leukaemic blast cells. DCs are being studied as a platform for the design of cancer vaccines. When the immune system recognizes the tumor, tumor-associated antigens (TAAs) are internalized, processed and presented by the antigen-presenting cells (APCs) as antigenic epitope peptides in the context of human leukocyte antigen (HLA) molecules (Lanzavecchia, 2001 #5). However, when the DCs are incubated with tumor antigens, the complexes of MHC class I molecule and antigen (peptide) may only be on the cell surface for a few hours. Thus, brief tumor antigen presentation by DCs in vivo has been a major problem in vaccine efficacy.

Recently, another novel gene that is highly expressed in many types of cancer was identified. This gene, named STEAP for six-transmembrane epithelial antigen of the prostate, is found in multiple cancers, including prostate, bladder, colon, ovarian, and Ewing sarcoma. The discovery of immunogenic peptides derived from STEAP is innovative and holds great promise. STEAP may be an ideal target for T-cell-mediated immunotherapy of advanced cancer, as STEAP is highly expressed at all stages of many cancers, including metastases; there is little or no expression of STEAP in normal human tissues; STEAP has cell surface localization and predicted secondary structure; and STEAP is not modulated by hormones, a property that is beneficial when managing hormone-refractory prostate cancer or during anti-androgen therapy for advanced metastatic disease. Protein analysis located STEAP at the cell surface of prostate cancer cells. Its strong expression in many cancers, little or no expression in normal tissues, and cell surface localization suggest that STEAP may be an ideal target for the immunotherapy of cancer.

Accordingly, in one embodiment, the invention provides immunogenic STEAP-derived peptide sequences that can be used for therapy of a variety of cancers. STEAP-specific CTL were also generated in vitro by direct immunization of blood cells from healthy volunteers and from patients with cancer. The STEAP-specific CTL were found to kill STEAP-expressing cancer cells in vitro. In addition, the invention demonstrates further enhancement of the immunogenicity of these peptides by specific modifications of their sequence.

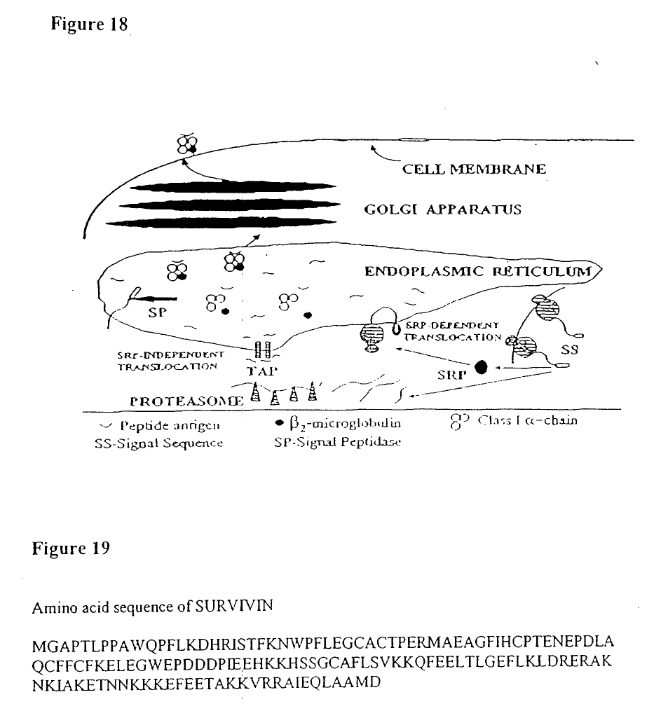

CTL play an important role in eradicating tumor cells and virus-infected cells. Unlike antibodies, which bind foreign proteins in their native form, CTL recognize short fragments of intracellular antigens, 8-10 amino acids in length, complexed with MHC Class I molecules. Cytosolic peptides are transported across the endoplasmic reticulum (ER) membrane with the help of the ATP-dependent transporters associated with antigen processing (TAP). Peptides complexed with Class I molecules in the ER are then transported to the cell surface for recognition by CTL. Studies with cell lines with deficits in antigen processing, (e.g., human T2 and murine RMA-S) have confirmed that TAP proteins are intimately involved in peptide transport. Alternatively, the translocation of processed proteins from the cytosol across the endoplasmic reticulum (ER) membrane is accomplished by endoplasmic reticulum-insertion signal sequences. As soon as the signal sequence of a growing polypeptide chain has emerged from the ribosome, it is bound by the signal recognition particle (SRP) and the complex is specifically targeted to the ER membrane by an interaction with the membrane bound SRP receptor (FIG. 18). An additional targeting pathway is the signal sequence receptor complex, which is a major protein of the eukaryotic ER membrane. While translocation usually occurs during translation, protein precursors have also been shown to be imported into the ER after their synthesis has been completed. After translocation, peptides complexed with class I molecules in the ER are transported to the cell surface for recognition by the CTL.

The T cell epitopes identified in the invention were utilized to construct fusion peptides with natural or artificial signal sequences. The effectiveness of the following signal sequences were compared in improving the antigen presentation: a) one from early region 3 of the adenovirus type 2, b) one from interferon gamma and c) several artificial sequences, generated according to the structure and the distribution frequency of the amino acids in the natural signal sequences. Since the hydrophobicity of the fusion peptides is higher than that of the minimal peptide, a set of control fusion peptides with signal sequences situated on the carboxy-terminus of the minimal peptides was used. Thus, determination was possible of whether an improved immune response generated with fusion peptides is due only to the higher hydrophobicity of the fusion peptide, or it is related to a better translocation of the minimal peptide through the ER-membrane. Since signal sequences do not contain specific amino acid residues other than a hydrophobic region of about eight residues, it was tested whether replacing this region with the hydrophobic HER2/neu-derived peptides would result in a more efficient presentation of these epitopes. To study the generality of the signal sequence approach similar constructs were designed utilizing several HER2/neu-derived peptides. The amino-acid sequences of the synthetic peptide constructs utilizing the epitopes HER2/neu₄₈₋₅₆, HER2/neu₃₆₉₋₃₇₇, HER2/neu₆₅₄₋₆₆₂, and HER2/neu₇₈₉₋₇₉₇ are shown in tables 32-35.

In one embodiment, the invention peptides are administered to a subject as fusion peptides containing a signal sequence. The PRAME-derived, OFA/iLRP derived, STEAP-derived, or SURVIVIN-derived peptide antigen is attached to, or incorporated into a synthetic insertion signal sequence, which can improve the translocation of the peptide antigen into the ER. Such fusion peptides can be used to treat patients with cancer by the following approaches: a) Patients can be immunized with fusion peptides composed of natural or artificial signal sequences and tumor-associated or viral peptide antigens. This way it might be possible to generate specific T-cell responses against the tumor and especially micro-metastases; b) Another way of practicing this invention is to load these peptide constructs into professional antigen-presenting cells and treat patients with these cells. This approach offers the advantage of having the specific antigen presented to the T-cells for a long period of time in the context of appropriate MHC molecules; and c) Patients can be treated with autologous CTL generated in vitro with fusion peptide-loaded dendritic cells or other antigen-presenting cells.

The term “signal sequence,” as used herein, refers to a short amino acid sequence added to an end of an antigenic peptide, or incorporating an antigenic peptide. This modification allows transfer of the antigenic peptide through membranes such as the ER or the cell membrane. The signal sequence is cleaved after the polypeptide has crossed the membrane.

The term “subject” as used herein refers to any individual or patient to which the subject methods are performed. Generally the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

As used herein, the term “treating” means that the clinical signs and/or the symptoms associated with the cancer or melanoma are lessened as a result of the actions performed. The signs or symptoms to be monitored will be characteristic of a particular cancer or melanoma and will be well known to the skilled clinician, as will the methods for monitoring the signs and conditions. For example, the skilled clinician will know that the size or rate of growth of a tumor can monitored using a diagnostic imaging method typically used for the particular tumor (e.g., using ultrasound or magnetic resonance image (MRI) to monitor a tumor).

Immunization with such fusion peptides may be used both for prevention and for treatment of tumors expressing specific tumor antigens. As more specific tumor antigens are revealed, this approach may provide a model for development of more effective vaccines for lung cancer, prostate cancer, melanoma, breast cancer and other tumors. This strategy of immunization may also be useful for eliciting CTL responses against viral diseases. The use of common HLA Class I molecules, such as HLA-A2 may make it possible to immunize a large proportion of patients by this strategy. Moreover, the ability to immunize against a minimal peptide, as opposed to complete proteins, may eliminate cross-reactivity with self-antigens or other highly homologous proteins.

The term “cancer” as used herein, includes any malignant tumor including, but not limited to, carcinoma and sarcoma. Cancer arises from the uncontrolled and/or abnormal division of cells that then invade and destroy the surrounding tissues. As used herein, “proliferating” and “proliferation” refer to cells undergoing mitosis. As used herein, “metastasis” refers to the distant spread of a malignant tumor from its sight of origin. Cancer cells may metastasize through the bloodstream, through the lymphatic system, across body cavities, or any combination thereof. The term “cancerous cell” as provided herein, includes a cell afflicted by any one of the cancerous conditions provided herein. Thus, the methods of the present invention include treatment of benign overgrowth of melanocytes, glia, brain tumors, prostate cancer, breast cancer, and lung cancer. The term “carcinoma” refers to a malignant new growth made up of epithelial cells tending to infiltrate surrounding tissues, and to give rise to metastases.

Accordingly, in one embodiment, the invention provides fusion peptides composed of insertion signal sequences and peptides derived from the breast cancer antigen HER2/neu. The fusion peptides improve antigen presentation and induce antitumor CTL with higher efficiency against breast cancer. The addition of a synthetic signal sequence at the NH₂-terminus, but not at the COOH-terminus, of the HER2/neu epitopes greatly enhanced their presentation in T2 target cells, breast cancer cells and dendritic, cells. Importantly, peptide constructs, composed of the HER2/neu epitopes replacing the hydrophobic part of the signal sequences were the most effective. The efficiency of the signal sequences in facilitating the HER2/neu peptide presentation was confirmed also by using cytokine-release assays. The mechanisms involved in the enhancement of antigen presentation by the fusion peptides proved that the effective presentation of the loaded peptide constructs is a result of their efficient loading into the cytosol and not simple binding to the surface HLA molecules.

By “loading” of the fusion peptides into the cytosol of T2 cells, cancer cells and dendritic cells is meant use of a technology called “osmotic lysis of pinocytic vesicles.” T2 cells were exposed to hypertonic medium containing the peptide constructs. Pinocytic vesicles form in this medium, and because of their increased internal osmotic pressure, they break in the cytosol when the cells are placed in hypotonic culture medium. The invention is based on a hypothesis that the signal sequence will translocate the minimal tumor-specific peptide from the cytosol into the ER, improving its presentation to CTL.

HER2/neu proto-oncogene, expressed in breast cancer and other human cancers, encodes a tyrosine kinase with homology to epidermal growth factor receptor. HER2/neu protein is a receptor-like transmembrane protein comprising a large cysteine-rich extracellular domain that functions in ligand binding, a short transmembrane domain, and a small cytoplasmic domain. HER2/neu is amplified and expressed in many human cancers, largely adenocarcinomas of breast, ovary, colon, and lung. In breast cancer, HER2/neu over-expression is associated with aggressive disease and is an independent predictor of poor prognosis. HER/neu is considered a possible target for T-cell-mediated immunotherapy for several reasons: (i) the protein is large (1255 amino acids), contains epitopes appropriate for binding to most MHC molecules and thus is potentially recognizable by all individuals; (ii) HER2/neu is greatly over-expressed on malignant cells and thus T-cell therapy may be selective with minimal toxicity; (iii) the oncogenic protein is intimately associated with the malignant phenotype and with the aggressiveness of the malignancy, especially in breast and ovarian carcinomas.

As shown in the Examples below, peptide signal sequences could improve presentation of the human tumor antigen HER2/neu. Since the transport of antigenic peptides from the cytosol to the endoplasmic reticulum (ER) is a limiting step in the processing of Class I-restricted antigens, bypassing this step of antigen processing is a clear advantage, resulting in more effective generation of CTL specifically directed against human cancers and viral diseases.

Signal sequences consist of three regions with specific characteristics shared by both eukaryotes and prokaryotes: (i) basic N-terminal region (n-region, pre-core, 1-3 positively charged residues); (ii) central hydrophobic region (h-region, core, 8-12 hydrophobic residues); and (iii) polar C-terminal region (c-region, post-core, 5-7 residues with higher average polarity than the h-region). The central hydrophobic region is the true hallmark of the signal sequences.

The primary structure is not critical to signal sequence functions. Comparison to all known signal sequences reveals no regions of strict homology. The cleavage site shows the strongest conservation, probably because it must be recognized by the signal peptidase.

Accordingly, the present invention provides peptide constructs composed of signal sequences, situated on the amino-terminus or the carboxy-terminus of several HER2/neu-derived peptides. In addition, the invention provides fusion peptides composed of natural or artificial signal sequences and HER2/neu peptides, replacing the hydrophobic part of the signal sequences.

Natural Signal Sequences:

| | |
|---|---|
| E3/19 adenoviral signal sequence: | (SEQ ID NO: 68) |
| MRYMILGLLALAAVCSA | |
| Gamma interferon signal sequence: | (SEQ ID NO: 69) |
| MTNKCLLQIALLLCFSTTALS | |

In performance of the present invention, the hydrophobic region of some signal sequences (natural and artificial) was replaced. Where this was performed, the following was noted: signal sequences do not contain specific amino acid residues other than a hydrophobic region of 8-12 residues; cleavage usually occurs after a small non-polar residue, which is the case with Val in position 9; Ala is the most abundant residue, associated with the cleavage site; the spacer of five Ala residues contributes to the predicted β-turn, which is found immediately before or after the cleavage site. The β-turn is thought to be important for peptidase access to the cleavage site.

In one embodiment, protein signal sequences are fused to HLA Class I-restricted minimal peptides for the development of synthetic vaccines against neoplastic and viral diseases. Immunizing with minimal determinant constructs may avoid the possible oncogenic effect of full-length proteins containing ras, p53 or other potential oncogenes. In addition, in vivo or in vitro immunization with peptide antigens “packaged” in dendritic cells or other antigen-presenting cells opens an exciting opportunity for eliciting powerful CTL-responses.

In another embodiment, the invention provides vaccines containing one or more fusion peptides as set forth above. The new vaccines can be used in subjects with advanced metastatic cancers, which are normally resistant to the conventional methods for treatment. Other cancers for which the synthetic vaccines are useful include, but are not limited to, melanoma, gliomas (Schwannoma, glioblastoma, astrocytoma), prostate cancer, renal cancer, breast cancer, lung cancer, acute leukemias, and many other cancers expressing known tumor-associated antigens. Dendritic cells loaded with these vaccines can also be used to elicit powerful anti-tumor immune responses in patients with cancer. In addition, fusion-peptide induced CTL might be extremely useful for cellular immunotherapy of cancer. This new approach may also be used to induce potent anti-viral immune responses.

All of the above-mentioned approaches can be applied using combinations of different tumor-associated or viral peptide antigens. This may allow generation of broader immune responses against the tumor or the virus-infected cell(s).

In another aspect, the methods of the invention are useful for providing a means for practicing personalized medicine, wherein treatment is tailored to a subject based on the particular characteristics of the cancer cells in the subject. The method can be practiced, for example, by contacting a sample of cells from the subject with at least one test peptide, wherein an increase in CTL in the presence of the test peptide as compared to CTL in the absence of the test peptide identifies the peptide as useful for treating the cancer. The sample of cells examined according to the present method can be obtained from the subject to be treated, or can be cells of an established cancer cell line of the same type as that of the subject. In one aspect, the established cancer cell line can be one of a panel of such cell lines, wherein the panel can include different cell lines of the same type of cancer and/or different cell lines of different cancers. Such a panel of cell lines can be useful, for example, to practice the present method when only a small number of cancer cells can be obtained from the subject to be treated, thus providing a surrogate sample of the subject's cancer, and also can be useful to include as control samples in practicing the present methods.

Preferred cell types for use in the invention include, but are not limited to, mammalian cells, including animal (rodents, including mice, rats, hamsters and gerbils), primates, and human cells, particularly cancer cells of all types, including breast, skin, lung, cervix, colorectal, leukemia, brain, etc.

As used herein, the terms “sample” and “biological sample” refer to any sample suitable for the methods provided by the present invention. In one embodiment, the biological sample of the present invention is a tissue sample, e.g., a biopsy specimen such as samples from needle biopsy. In other embodiments, the biological sample of the present invention is a sample of bodily fluid, e.g., serum, plasma, urine, and ejaculate.

Once disease is established and a treatment protocol is initiated, the methods of the invention may be repeated on a regular basis to evaluate whether the level of peptide-specific CTL activity in the subject remains elevated as compared to that which is observed in a normal subject. The results obtained from successive assays may be used to show the efficacy of treatment over a period ranging from several days to months. Accordingly, the invention is also directed to methods for monitoring a therapeutic regimen for treating a subject having cancer. A comparison of the peptide-specific CTL activity prior to and during therapy indicates the efficacy of the therapy. Therefore, one skilled in the art will be able to recognize and adjust the therapeutic approach as needed.

In certain embodiments, the invention nanoparticles may further be administered in combination with a therapeutic agent. Exemplary therapeutic agents include, but are not limited to anti-inflammatory agents, antimicrobial agents, antihistamines, chemotherapeutic agents, antiangiogenic agents, immunomodulators, therapeutic antibodies or protein kinase inhibitors, e.g., a tyrosine kinase inhibitor. Other therapeutic agents that may be administered in combination with invention nanoparticles include protein therapeutic agents such as cytokines, immunomodulatory agents, anticancer agents and antibodies. While not wanting to be limiting, antimicrobial agents include antivirals, antibiotics, anti-fungals and anti-parasitics. When other therapeutic agents are employed in combination with the nanoparticles of the present invention, they may be used for example in amounts as noted in the Physician Desk Reference (PDR) or as otherwise determined by one having ordinary skill in the art.

As used herein the term “cytokine” encompasses chemokines, interleukins, lymphokines, monokines, colony stimulating factors, and receptor associated proteins, and functional fragments thereof. Exemplary cytokines include, but are not limited to, endothelial monocyte activating polypeptide II (EMAP-II), granulocyte-macrophage-CSF (GM-CSF), granulocyte-CSF (G-CSF), macrophage-CSF (M-CSF), IL-1, IL-2, IL-3, IL-4, IL-5, IL-6, IL-12, and IL-13, interferons, and the like and which is associated with a particular biologic, morphologic, or phenotypic alteration in a cell or cell mechanism.

All methods may further include the step of bringing the active ingredient(s) into association with a pharmaceutically acceptable carrier, which constitutes one or more accessory ingredients. The term “pharmaceutically acceptable”, when used in reference to a carrier, is meant that the carrier, diluent or excipient must be compatible with the other ingredients of the formulation and not deleterious to the recipient thereof.

Pharmaceutically acceptable carriers useful for formulating a peptide or synthetic vaccine for administration to a subject are well known in the art and include, for example, aqueous solutions such as water or physiologically buffered saline or other solvents or vehicles such as glycols, glycerol, oils such as olive oil or injectable organic esters. A pharmaceutically acceptable carrier can contain physiologically acceptable compounds that act, for example, to stabilize or to increase the absorption of the conjugate. Such physiologically acceptable compounds include, for example, carbohydrates, such as glucose, sucrose or dextrans, antioxidants, such as ascorbic acid or glutathione, chelating agents, low molecular weight proteins or other stabilizers or excipients. One skilled in the art would know that the choice of a pharmaceutically acceptable carrier, including a physiologically acceptable compound, depends, for example, on the physico-chemical characteristics of the therapeutic agent and on the route of administration of the composition, which can be, for example, orally or parenterally such as intravenously, and by injection, intubation, or other such method known in the art. The pharmaceutical composition also can contain a second (or more) compound(s) such as a diagnostic reagent, nutritional substance, toxin, or therapeutic agent, for example, a cancer chemotherapeutic agent and/or vitamin(s).

The peptides and peptide vaccines of the invention can be incorporated within an encapsulating material such as into an oil-in-water emulsion, a microemulsion, a micelle, mixed micelle, a liposome, a microsphere, a polymeric nanoparticle, or other polymer matrix (see, for example, Gregoriadis, Liposome Technology, Vol. 1 (CRC Press, Boca Raton, Fla. 1984); Fraley, et al., Trends Biochem. Sci., 6:77 (1981), each of which is incorporated herein by reference).

Liposomes, for example, which consist of phospholipids or other lipids, are nontoxic, physiologically acceptable and metabolizable carriers that are relatively simple to make and administer. “Stealth” liposomes (see, for example, U.S. Pat. Nos. 5,882,679; 5,395,619; and 5,225,212, each of which is incorporated herein by reference) are an example of such encapsulating materials particularly useful for preparing a pharmaceutical composition useful for practicing a method of the invention, and other “masked” liposomes similarly can be used, such liposomes extending the time that the therapeutic agent remain in the circulation. Cationic liposomes, for example, also can be modified with specific receptors or ligands (Morishita et al., J. Clin. Invest. 91:2580-2585 (1993), which is incorporated herein by reference). In addition, a polynucleotide agent can be introduced into a cell using, for example, adenovirus-polylysine DNA complexes (see, for example, Michael et al., J. Biol. Chem. 268:6866-6869 (1993), which is incorporated herein by reference).

Nanotechnology can be used to formulate therapeutic agents in biocompatible nanocomposites such as nanoparticles, nanocapsules, micellar systems, and conjugates (Panyam, 2003 #15). Nanoparticles are submicron-sized (e.g., in the range of 10-1000 nm) polymeric colloidal particles. Polymeric nanoparticles allow encapsulation of the peptides or peptide vaccines inside a polymeric matrix, protecting them against enzymatic and hydrolytic degradation. Thus, a therapeutic agent of interest can be encapsulated within their polymeric matrix or adsorbed or conjugated onto the surface (Labhasetwar, 1997 #16). Biodegradable nanoparticles generated from poly(D,L-lactide-co-glycolide) (PLGA) have recently attracted substantial attention because of their clinically proven biocompatibility (Foged, 2002 #17; Little, 2004 #18). These kinds of PLGA nanoparticles have recently been proposed as a potential antigen delivery vehicle for DC-based vaccines against pathogens and cancer (Waeckerle-Men, 2005 #19). In addition, the nanoparticle-vaccine approach provides the ability to customize various properties of the vaccine materials that may improve their function. Such variable properties include, but are not limited to, particle size, pH sensitivity, surface charge, and hydrophobicity.

PLGA nanoparticles not only facilitate the uptake of encapsulated peptides, proteins and DNA, but also potentially can protect peptides, nucleic acids, and protein antigens contained within from extracellular degradation, and therefore increase their delivery efficiency (Panyam, 2003 #20). Useful characteristics of PLGA nanoparticles such as in vivo biodegradability, an adjustable release profile, and the very high encapsulation capacity have stimulated immunologists to explore PLGA nanoparticles as antigen delivery systems for vaccination. In this study, biodegradable PLGA nanoparticles are used to develop human DC-based vaccine with enhanced and prolonged antigen presentation.

Based on previous results, sustained release of the target drug/DNA inside poly(D,L-lactide-co-glycolide) (PLGA) nanoparticles was observed in human umbilical vein endothelial cells (Davda, 2002 #22), prostate cancer cells (Sahoo, 2004 #24), and vascular smooth muscle cells (Panyam, 2003 #25). A double emulsion-solvent evaporation technique was employed to make the PLGA nanoparticles with a size around 220 nm. It was tested whether this characteristic could prolong antigen levels within human DCs for periods surpassing other antigen forms. In this study, PLGA-based nanoparticles were used as a delivery system to carry the tumor antigen Mart-1:27-35 peptide into human DCs in order to acquire prolonged antigen presentation. It was demonstrated that 100% of imDCs phagocytosed NPs after incubating them for just 1 hour with the NP suspension (100 μg/ml). In order to induce an immune response, DCs have to internalize the nanoparticles containing Mart-1:27-35 peptide. Phagocytosis of the nanoparticles was demonstrated by incubating the PLGA nanoparticles containing the fluorescent dye coumarin 6 with imDCs at 37° C. for 1 h.

Based on the data presented herein, PLGA nanoparticles are believed to have a small direct effect on the maturation of human DCs. The results show that DCs exhibited a mild increase in the expression of MHC class II, CD80, CD86 and CD83 when compared with unpulsed controls. A similar result was observed using PLGA nanoparticle formulations and cord blood derived DCs (Diwan, 2003 #37), as well as murine bone marrow derived DCs (Elamanchili, 2004 #33).

Using the proposed PLGA nanoparticles containing Mart-1:27-35 peptide as a delivery system to human DCs offered distinct advantages over the administration of soluble Mart-1:27-35 peptide. The important benefits include: prevention of proteolytic degradation of antigen; higher efficiency of peptide loading; prolonged and enhanced antigen presentation by human DCs. These results are consistent with other reports (Audran, 2003 #36). Consequently, this PLGA formulation containing Mart-1: 27-35 peptide can enhance and prolong MHC class I antigen presentation. As PLGA is safe for humans and already approved by the FDA for clinical use, the present invention may have significant clinical potential in tumor immunotherapy.

The lack of efficient and long-lasting antigen presentation by DCs in vivo has been a major problem in vaccine efficacy. Human DCs have recently been developed for clinical use to treat patients with infections and malignant diseases. Various attempts to deliver tumor antigens to human DCs, as well as routes and schedules of administration to cancer patients, are currently being analyzed in clinical trials (Cerundolo, 2004 #11; Figdor, 2004 #10; Whiteside, 2004 #12). The method of antigen delivery to human DCs corresponds to the efficiency of antigen presentation and the resulting immune response. The loading methods for DCs can be categorized in the following manner: loading with peptides, proteins (receptor, lysosome, recombinant bacterial toxin, or peptide-mediated), whole tumor cells, tumor-derived exosomes, tumor derived RNA, tumor derived DNA (including viral vector mediated), and in vivo loading (Zhou, 2002 #13). Many attempts have been made to exogenously load peptides or tumor antigens onto the surface of human DCs. Unfortunately, the resulting MHC class I molecule/peptide complexes may only be presented on the cell surface for several hours, limiting the potential vaccine efficacy (Waeckerle-Men, 2005 #14). It therefore appears that this obstacle can be circumvented through the development of nanoparticles, which can efficiently deliver the antigenic peptides into the antigen presenting cells (APC).

The carriers, in addition to those disclosed above, can include glucose, lactose, mannose, gum acacia, gelatin, mannitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition auxiliary, stabilizing, thickening or coloring agents and perfumes can be used, for example a stabilizing dry agent such as triulose (see, for example, U.S. Pat. No. 5,314,695, which is incorporated herein by reference).

The route of administration of a composition containing the peptides of the invention will depend, in part, on the chemical structure of the molecule. As used herein, the terms “administration” or “administering” are defined to include an act of providing a compound or pharmaceutical composition of the invention to a subject in need of treatment. Polypeptides and polynucleotides, for example, are not particularly useful when administered orally because they can be degraded in the digestive tract. However, methods for chemically modifying polynucleotides and polypeptides, for example, to render them less susceptible to degradation by endogenous nucleases or proteases, respectively, or more absorbable through the alimentary tract are well known (see, for example, Blondelle et al., Trends Anal. Chem. 14:83-92, 1995; Ecker and Crook, BioTechnology, 13:351-360, 1995). For example, a peptide of the invention can be prepared using D-amino acids, or can contain one or more domains based on peptidomimetics, which are organic molecules that mimic the structure of peptide domain; or based on a peptoid such as a vinylogous peptoid. The peptides of the invention can further be administered in a form that releases the peptide at the desired position in the body (e.g., the stomach), or by injection into a blood vessel such that the peptide circulates to the target cells (e.g., cancer cells).

Exemplary routes of administration include, but are not limited to, orally or parenterally, such as intravenously, intramuscularly, subcutaneously, intraperitoneally, intrarectally, intracisternally or, if appropriate, by passive or facilitated absorption through the skin using, for example, a skin patch or transdermal iontophoresis, respectively. Furthermore, the pharmaceutical composition can be administered by injection, intubation, orally or topically, the latter of which can be passive, for example, by direct application of an ointment, or active, for example, using a nasal spray or inhalant, in which case one component of the composition is an appropriate propellant. As mentioned above, the pharmaceutical composition also can be administered to the site of a tumor, for example, intravenously or intra-arterially into a blood vessel supplying the tumor.

The total amount of a peptide or vaccine to be administered in practicing a method of the invention can be administered to a subject as a single dose, either as a bolus or by infusion over a relatively short period of time, or can be administered using a fractionated treatment protocol, in which multiple doses are administered over a prolonged period of time. One skilled in the art would know that the amount of peptide or synthetic vaccine to treat cancer in a subject depends on many factors including the age and general health of the subject as well as the route of administration and the number of treatments to be administered. In view of these factors, the skilled artisan would adjust the particular dose as necessary. In general, the formulation of the pharmaceutical composition and the routes and frequency of administration are determined, initially, using Phase I and Phase II clinical trials.

In general, a suitable daily dose of a compound or composition of the invention will be that amount of the compound or composition that is the lowest dose effective to produce a therapeutic effect. Such an effective dose will generally depend upon the factors described above. As used herein, the term “therapeutically effective amount” or “effective amount” means the amount of a compound or pharmaceutical composition that will elicit the biological or medical response of a tissue, system, animal or human that is being sought by the researcher, veterinarian, medical doctor or other clinician.

Additionally, the compositions and methods of the invention can be used in conjunction with other standard cancer therapies, e.g., surgery, chemotherapy and radiation.

In another aspect, the invention provides kits for performing the methods of the invention that include one or more nanoparticles of the invention. In one embodiment, the kit further includes one or more therapeutic agents for use with the nanoparticle of the invention. In another embodiment, the kit includes instructions for practicing the methods of the invention

The following examples are provided to further illustrate the advantages and features of the present invention, but are not intended to limit the scope of the invention. While they are typical of those that might be used, other procedures, methodologies, or techniques known to those skilled in the art may alternatively be used.

### EXAMPLE 1

### Enhancing the Stability, Immunogenicity, and Antigen Presentation of PRAME-Derived Synthetic Peptides

Most attempts to treat cancer patients with TAA-derived synthetic peptides have not been successful. The following is therefore further research aimed at enhancing the stability and immunogenicity of the peptides used for vaccination of patients with cancer is essential.

When biologically active peptides are used clinically in their natural form, their biologic effects are often rapidly lost in vivo due to rapid elimination of the active form of the peptide. Since the skin is an enzymatically active organ, in vaccinations that utilize subcutaneous injections, peptides may be degraded by skin peptidases prior to effecting a significant immunological response. Thus, it is critical to design stable peptide formulations for vaccination of patients with cancer. The natural HLA-A2.1 restricted PRAME peptides were modified by N-terminal acetylation and/or C-terminal amidation. Examples of modifications to the native HLA-A2.1 restricted PRAME peptides are shown (Tables 2-5):

TABLE 2

Terminal modifications of the
PRAME-derived peptide PRAME₁₀₀₋₁₀₈

| | | Modifications | |
|---|---|---|---|
| Peptide Name | Peptide Sequence | N-Terminus | C-Terminus |

| | | | |
|---|---|---|---|
| PRAME₁₀₀₋₁₀₈ | ¹⁰⁰VLDGLDVLL¹⁰⁸ | — | — |
| | (SEQ ID NO: 2) | | |
| N- PRAME₁₀₀₋₁₀₈ | Ac- VLDGLDVLL | Acetyl | — |
| | (SEQ ID NO: 6) | | |
| C- PRAME₁₀₀₋₁₀₈ | VLDGLDVLL -amide | — | Amide |
| | (SEQ ID NO: 7) | | |
| Cap- PRAME₁₀₀₋₁₀₈ | Ac- VLDGLDVLL -amide | Acetyl | Amide |
| | (SEQ ID NO: 8) | | |

TABLE 3

Terminal modifications of the
PRAME-derived peptide PRAME₁₄₂₋₁₅₁

| | | Modifications | |
|---|---|---|---|
| Peptide Name | Peptide Sequence | N-Terminus | C-Terminus |

| | | | |
|---|---|---|---|
| PRAME₁₄₂₋₁₅₁ | ¹⁴²SLYSFPEPEA¹⁵¹ | — | — |
| | (SEQ ID NO: 3) | | |
| N- PRAME₁₄₂₋₁₅₁ | Ac- SLYSFPEPEA | Acetyl | — |
| | (SEQ ID NO: 9) | | |
| C- PRAME₁₄₂₋₁₅₁ | SLYSFPEPEA -amide | — | Amide |
| | (SEQ ID NO: 10) | | |
| ap- PRAME₁₄₂₋₁₅₁ | Ac- SLYSFPEPEA -amide | Acetyl | Amide |
| | (SEQ ID NO: 11) | | |

TABLE 4

Terminal modifications of the
PRAME-derived peptide PRAME₃₀₀₋₃₀₉

| | | Modifications | |
|---|---|---|---|
| Peptide Name | Peptide Sequence | N-Terminus | C-Terminus |

| | | | |
|---|---|---|---|
| PRAME₃₀₀₋₃₀₉ | ³⁰⁰ALYVDSLFFL³⁰⁹ | — | — |
| | (SEQ ID NO: 4) | | |
| N- PRAME₃₀₀₋₃₀₉ | Ac- ALYVDSLFFL | Acetyl | — |
| | (SEQ ID NO: 12) | | |
| C- PRAME₃₀₀₋₃₀₉ | ALYVDSLFFL -amide | — | Amide |
| | (SEQ ID NO: 13) | | |
| Cap- PRAME₃₀₀₋₃₀₉ | Ac- ALYVDSLFFL -amide | Acetyl | Amide |
| | (SEQ ID NO: 14) | | |

TABLE 5

Terminal modifications of the
PRAME-derived peptide PRAME₄₂₅₋₄₃₃

| | | Modifications | |
|---|---|---|---|
| Peptide Name | Peptide Sequence | N-Terminus | C-Terminus |

| | | | |
|---|---|---|---|
| PRAME₄₂₅₋₅₃₃ | ⁴²⁵SLLQHLIGL⁴³³ | — | — |
| | (SEQ ID NO: 5) | | |
| N- PRAME₄₂₅₋₄₃₃ | Ac- SLLQHLIGL | Acetyl | — |
| | (SEQ ID NO: 15) | | |
| C- PRAME₄₂₅₋₄₃₃ | SLLQHLIGL -amide | — | Amide |
| | (SEQ ID NO: 16) | | |
| Cap- PRAME₄₂₅₋₄₃₃ | Ac- SLLQHLIGL -amide | Acetyl | Amide |
| | (SEQ ID NO: 17) | | |

Amino Acid Substitutions at HLA-A2.1 Binding Anchor Positions to Enhance MHC Class I Binding Affinity of the PRAME Peptides (Fixed Anchor Analogs):

Upon stimulation with natural peptides, tumor-reactive CTL have been induced in vitro from peripheral blood lymphocytes of some patients with cancer. However, tumor-specific CTL could only be induced in a limited number of patients, and numerous re-stimulations were required to generate anti-tumor reactivity. These findings prompted this section of the current invention aimed at enhancing the immunogenicity of peptides derived from PRAME.

As an example, the following anchor amino acid substitutions to the native HLA-A2.1 restricted PRAME peptides were created (Tables 6-9):

TABLE 6

Substitutions at the HLA-A2.1 binding anchor
positions of the peptide PRAME₁₀₀₋₁₀₈

| | | |
|---|---|---|
| Peptide Name | Peptide Sequence | Substitutions |

| | | |
|---|---|---|
| PRAME₁₀₀₋₁₀₈ | ¹⁰⁰VLDGLDVLL¹⁰⁸ | — |
| | (SEQ ID NO: 2) | |
| PRAME₁₀₀₋₁₀₈ -1F | FLDGLDVLL | F for V at position 1 |
| | (SEQ ID NO: 18) | |
| PRAME₁₀₀₋₁₀₈ -3W | VLWGLDVLL | W for D at position 3 |
| | (SEQ ID NO: 19) | |
| PRAME₁₀₀₋₁₀₈ -9V | VLDGLDVLV | V for L at position 9 |
| | (SEQ ID NO: 20) | |
| PRAME₁₀₀₋₁₀₈ -1F/3W/9V | FLWGLDVLV | all of the above |
| | (SEQ ID NO: 21) | |

TABLE 7

Substitutions at the HLA-A2.1 binding anchor
positions of the peptide PRAME₁₄₂₋₁₅₁

| | | |
|---|---|---|
| Peptide Name | Peptide Sequence | Substitutions |

| | | |
|---|---|---|
| PRAME₁₄₂₋₁₅₁ | ¹⁴²SLYSFPEPEA¹⁵¹ | — |
| | (SEQ ID NO: 3) | |
| PRAME₁₄₂₋₁₅₁ -1F | FLYSFPEPEA | F for S at position 1 |
| | (SEQ ID NO: 22) | |
| PRAME₁₄₂₋₁₅₁ -3W | SLWSFPEPEA | W for Y at position 3 |
| | (SEQ ID NO: 23) | |
| PRAME₁₄₂₋₁₅₁ -10V | SLYSFPEPEV | V for A at position 10 |
| | (SEQ ID NO: 24) | |
| PRAME₁₄₂₋₁₅₁ -1F/3W/10V | FLWSFPEPEV | all of the above |
| | (SEQ ID NO: 25) | |

TABLE 8

Substitutions at the HLA-A2.1 binding anchor
positions of the peptide PRAME₃₀₀₋₃₀₉

| | | |
|---|---|---|
| Peptide Name | Peptide Sequence | Substitutions |

| | | |
|---|---|---|
| PRAME₃₀₀₋₃₀₉ | ³⁰⁰ALYVDSLFFL³⁰⁹ | — |
| | (SEQ ID NO: 4) | |
| PRAME₃₀₀₋₃₀₉ -3W | ALFVDSLFFL | F for Y at position 3 |
| | (SEQ ID NO: 26) | |
| PRAME₃₀₀₋₃₀₉ -10V | ALYVDSLFFV | V for L at position 10 |
| | (SEQ ID NO: 27) | |
| PRAME₃₀₀₋₃₀₉ -3F/10V | ALFVDSLFFV | all of the above |
| | (SEQ ID NO: 28) | |

TABLE 9

Substitutions at the HLA-A2.1 binding anchor
positions of the peptide PRAME₄₂₅₋₄₃₃

| | | |
|---|---|---|
| Peptide Name | Peptide Sequence | Substitutions |

| | | |
|---|---|---|
| PRAME₄₂₅₋₄₃₃ | ⁴²⁵SLLQHLIGL⁴³³ | — |
| | (SEQ ID NO: 5) | |
| PRAME₄₂₅₋₄₃₃ -1F | FLLQHLIGL | F for S at position 1 |
| | (SEQ ID NO: 29) | |
| PRAME₄₂₅₋₄₃₃ -3W | SLWQHLIGL | W for L at position 3 |
| | (SEQ ID NO: 30) | |
| PRAME₄₂₅₋₄₃₃ -9V | SLLQHLIGV | V for L at position 9 |
| | (SEQ ID NO: 31) | |
| PRAME₄₂₅₋₄₃₃ -1F/3W/9V | FLWQHIIGV | all of the above |
| | (SEQ ID NO: 32) | |

Amino Acid Substitutions at NON-Anchor Positions to Enhance the T Cell Receptor Binding Affinity for the Peptide-MHC Complex (Heteroclitic Analogs)

Certain peptide analogs that carry amino acid substitutions at residues other than the main MHC anchors (heteroclitic analogs) have shown a significantly increased potency, and are surprisingly much more antigenic than wild-type peptides. These analogs may provide considerable benefit in vaccine development, as they induce stronger T cell responses than the native epitope, and have been shown to be associated with increased affinity of the epitope/MHC complex for the T cell receptor (TCR) molecule. Important advantages of the heteroclitic analogs related to their clinical application include their ability to break/overcome tolerance by reversing a state of T cell energy and/or recruiting new T cell specificities, and the significantly smaller amounts of heteroclitic analogs that is needed for treatment.

The scheme that used for selection of the single amino acid substitutions includes rank coefficient scores for PAM250, hydrophobicity, and side chain volume. The Dayhoff PAM250 score (hyper text transfer protocol address prowl.rockefeller.edu/aainfo/pam250.htm) is a commonly used protein alignment scoring matrix which measures the percentage of acceptable point mutations within a defined time frame.

The following NON-anchor amino acid substitutions were made to the native HLA-A2.1 restricted PRAME peptides (Tables 10-13):

TABLE 10

Substitutions at NON-anchor positions
of the peptide PRAME₁₀₀₋₁₀₈

| | | |
|---|---|---|
| Peptide Name | Peptide Sequence | Substitutions |

| | | |
|---|---|---|
| PRAME₁₀₀₋₁₀₈ | ¹⁰⁰VLDGLDVLL¹⁰⁸ | — |
| | (SEQ ID NO: 2) | |
| PRAME₁₀₀₋₁₀₈ - 3K | VLKGLDVLL | K for D at |
| | (SEQ ID NO: 33) | position 3 |
| PRAME₁₀₀₋₁₀₈ - 5H | VLDGHDVLL | H for L at |
| | (SEQ ID NO: 34) | position 5 |
| PRAME₁₀₀₋₁₀₈ - 7P | VLDGLDPLL | P for V at |
| | (SEQ ID NO: 35) | position 7 |

TABLE 11

Substitutions at NON-anchor positions
of the peptide PRAME₁₄₂₋₁₅₁

| | | |
|---|---|---|
| Peptide Name | Peptide Sequence | Substitutions |

| | | |
|---|---|---|
| PRAME₁₄₂₋₅₁ | ¹⁴² SLYSFPEPEA¹⁵¹ | — |
| | (SEQ ID NO: 3) | |
| PRAME₁₄₂₋₁₅₁ - 3K | SLKSFPEPEA | K for Y at |
| | (SEQ ID NO: 36) | position 3 |
| PRAME₁₄₂₋₁₅₁ - 5H | SLYSHPEPEA | H for F at |
| | (SEQ ID NO: 37) | position 5 |
| PRAME₁₄₂₋₁₅₁ - 7P | SLYSFPPPEA | P for B at |
| | (SEQ ID NO: 38) | position 7 |

TABLE 12

Substitutions at NON-anchor positions
of the peptide PRAME₃₀₀₋₃₀₉

| | | |
|---|---|---|
| Peptide Name | Peptide Sequence | Substitutions |

| | | |
|---|---|---|
| PRAME₃₀₀₋₃₀₉ | ³⁰⁰ALYVDSLFFL³⁰⁹ | — |
| | (SEQ ID NO: 4) | |
| PRAME₃₀₀₋₃₀₉ - 3K | ALKVDSLFFL | K for Y at |
| | (SEQ ID NO: 39) | position 3 |
| PRAME₃₀₀₋₃₀₉ - 5H | ALYVHSLFFL | H for D at |
| | (SEQ ID NO: 40) | position 5 |
| PRAME₃₀₀₋₃₀₉ - 7P | ALYVDSPFFL | P for L at |
| | (SEQ ID NO: 41) | position 7 |

TABLE 13

Substitutions at NON-anchor positions
of the peptide PRAME₄₂₅₋₄₃₃

| | | |
|---|---|---|
| Peptide Name | Peptide Sequence | Substitutions |

| | | |
|---|---|---|
| PRAME₄₂₅₋₄₃₃ | ⁴²⁵SLLQHLIGL⁴³³ | — |
| | (SEQ ID NO: 5) | |
| PRAME₄₂₅₋₄₃₃ - 3K | SLKQHLIGL | K for L at |
| | (SEQ ID NO: 42) | position 3 |
| PRAME₄₂₅₋₄₃₃ - 7P | SLLQHLPGL | P for I at |
| | (SEQ ID NO: 43) | position 7 |

Enhancing the Immunogenicity of the Peptides with Insertion Signal Sequences

The transport of antigenic peptides from the cytosol to the endoplasmic reticulum (ER) is a limiting step in processing and presentation of class I-restricted antigens. Bypassing this step by direct targeting of the antigen to the ER can result in more effective generation of CTL. This could amount to a more potent CTL induction and anti-tumor immunity against cancer. A variety of fusion peptides composed of natural or modified PRAME peptides and endoplasmic reticulum insertion signal sequences were designed. The following signal sequences were utilized to improve the antigen presentation: a) one from early region 3 of the adenovirus type 2—ES (MRYMILGLLALAAVCSA) (SEQ ID NO: 68), b) one from IFN-beta—IS (MTNKCLLQIALLLCFSTTALS) (SEQ ID NO: 69), and c) several artificial sequences, generated according to the structure and the distribution frequency of the amino acids in the natural signal sequences. Examples of synthetic peptide constructs utilizing the PRAME epitopes are shown (Tables 14-17).

TABLE 14
Synthetic peptide constructs utilizing the epitope PRAME₁₀₀₋₁₀₈

| | |
|---|---|
| Designation | Peptide Sequence |

| | |
|---|---|
| 1. PRAME - PRAME₁₀₀₋₁₀₈ | VLDGLDVLL |
| | (SEQ ID NO: 2) |
| 2. ES-PRAME | M R Y M I L G L L A L A A V C S A VLDGLDVLL |
| | (SEQ ID NO: 44) |
| 3. PRAME-ES | VLDGLDVLL M R Y M I L G L L A L A A V C S A |
| | (SEQ ID NO: 45) |
| 4. IS-PRAME | M T N K C L L Q I A L L L C F S T T A L S VLDGLDVLL |
| | (SEQ ID NO: 46) |
| 5. PRAME-IS | VLDGLDVLL M T N K C L L Q I A L L L C F S T T A L S |
| | (SEQ ID NO: 47) |
| 6. PRAME-IN-ES | M R VLDGLDVLL A A V C S A |
| | (SEQ ID NO: 48) |
| 7. PRAME-IN-AF | M A VLDGLDVLL A A A A A G |
| | (SEQ ID NO: 49) |

Synthetic peptide constructs:
1. Peptide antigen PRAME₁₀₀₋₁₀₈
2. Adenoviral signal sequence ES attached to the amino-terminus of PRAME₁₀₀₋₁₀₈
3. Adenoviral signal sequence ES attached to the carboxy-terminus of PRAME₁₀₀₋₁₀₈
4. Interferon signal sequence IS attached to the amino-terminus of PRAME₁₀₀₋₁₀₈
5. Interferon signal sequence IS attached to the carboxy-terminus of PRAME₁₀₀₋₁₀₈
6. Peptide antigen PRAME₁₀₀₋₁₀₈ replacing the hydrophobic portion of ES
7. Peptide antigen PRAME₁₀₀₋₁₀₈ incorporated into an artificial signal sequence - AF

TABLE 15
Synthetic peptide constructs utilizing the epitope PRAME₁₄₂₋₁₅₁

| | |
|---|---|
| Designation | Peptide Sequence |

| | |
|---|---|
| 1. PRAME - PRAME₁₄₂₋₁₅₁ | SLYSFPEPEA |
| | (SEQ ID NO: 3) |
| 2. ES-PRAME | M R Y M I L G L L A L A A V C S A SLYSFPEPEA |
| | (SEQ ID NO: 50) |
| 3. PRAME-ES | SLYSFPEPEA M R Y M I L G L L A L A A V C S A |
| | (SEQ ID NO: 51) |
| 4. IS-PRAME | M T N K C L L Q I A L L L C F S T T A L S SLYSFPEPEA |
| | (SEQ ID NO: 52) |
| 5. PRAME-IS | SLYSFPEPEA M T N K C L L Q I A L L L C F S T T A L S |
| | (SEQ ID NO: 53) |
| 6. PRAME-IN-ES | M R SLYSFPEPEA A A V C S A |
| | (SEQ ID NO: 54) |
| 7. PRAME-IN-AF | M A SLYSFPEPEA A A A A A G |
| | (SEQ ID NO: 55) |

Synthetic peptide constructs:
1. Peptide antigen PRAME₁₄₂₋₁₅₁
2. Adenoviral signal sequence ES attached to the amino-terminus of PRAME₁₄₂₋₁₅₁
3. Adenoviral signal sequence ES attached to the carboxy-terminus of PRAME₁₄₂₋₁₅₁
4. Interferon signal sequence IS attached to the amino-terminus of PRAME₁₄₂₋₁₅₁
5. Interferon signal sequence IS attached to the carboxy-terminus of PRAME₁₄₂₋₁₅₁
6. Peptide antigen PRAME₁₄₂₋₁₅₁ replacing the hydrophobic portion of ES
7. Peptide antigen PRAME₁₄₂₋₁₅₁ incorporated into an artificial signal sequence - AF

TABLE 16
Synthetic peptide constructs utilizing the epitope PRAME₃₀₀₋₃₀₉

| | |
|---|---|
| Designation | Peptide Sequence |

| | |
|---|---|
| 1. PRAME - PRAME₃₀₀₋₃₀₉ | ALYVDSLFFL |
| | (SEQ ID NO: 4) |
| 2. ES-PRAME | M R Y M I L G L L A L A A V C S A ALYVDSLFFL |
| | (SEQ ID NO: 56) |
| 3. PRAME-ES | ALYVDSLFFL M R Y M I L G L L A L A A V C S A |
| | (SEQ ID NO: 57) |
| 4. IS-PRAME | M T N K C L L Q I A L L L C F S T T A L S ALYVDSLFFL |
| | (SEQ ID NO: 58) |
| 5. PRAME-IS | ALYVDSLFFL M T N K C L L Q I A L L L C F S T T A L S |
| | (SEQ ID NO: 59) |
| 6. PRAME-IN-ES | M R ALYVDSLFFL A A V C S A |
| | (SEQ ID NO: 60) |
| 7. PRAME-IN-AF | M A ALYVDSLFFL A A A A A G |
| | (SEQ ID NO: 61) |

Synthetic peptide constructs:
1. Peptide antigen PRAME₃₀₀₋₃₀₉
2. Adenoviral signal sequence ES attached to the amino-terminus of PRAME₃₀₀₋₃₀₉
3. Adenoviral signal sequence ES attached to the carboxy-terminus of PRAME₃₀₀₋₃₀₉
4. Interferon signal sequence IS attached to the amino-terminus of PRAME₃₀₀₋₃₀₉
5. Interferon signal sequence IS attached to the carboxy-terminus of PRAME₃₀₀₋₃₀₉
6. Peptide antigen PRAME₃₀₀₋₃₀₉ replacing the hydrophobic portion of ES
7. Peptide antigen PRAME₃₀₀₋₃₀₉ incorporated into an artificial signal sequence - AF

TABLE 17
Synthetic peptide constructs utilizing the epitope PRAME425-433

| | |
|---|---|
| Designation | Peptide Sequence |

| | |
|---|---|
| 1. PRAME - PRAME₄₂₅₋₄₃₃ | SLLQHLIGL |
| | (SEQ ID NO: 5) |
| 2. ES-PRAME | M R Y M I L G L L A L A A V C S A SLLQHLIGL |
| | (SEQ ID NO: 62) |
| 3. PRAME-ES | SLLQHLIGL M R Y M I L G L L A L A A V C S A |
| | (SEQ ID NO: 63) |
| 4. IS-PRAME | M T N K C L L Q I A L L L C F S T T A L S SLLQHLIGL |
| | (SEQ ID NO: 64) |
| 5. PRAME-IS | SLLQHLIGL M T N K C L L Q I A L L L C F S T T A L S |
| | (SEQ ID NO: 65) |
| 6. PRAME-IN-ES | M R SLLQHLIGL A A V C S A |
| | (SEQ ID NO: 66) |
| 7. PRAME-IN-AF | M A SLLQHLIGL A A A A A G |
| | (SEQ ID NO: 67) |

Synthetic peptide constructs:
1. Peptide antigen PRAME₄₂₅₋₄₃₃
2. Adenoviral signal sequence ES attached to the amino-terminus of PRAME₄₂₅₋₄₃₃
3. Adenoviral signal sequence ES attached to the carboxy-terminus of PRAME₄₂₅₋₄₃₃
4. Interferon signal sequence IS attached to the amino-terminus of PRAME₄₂₅₋₄₃₃
5. Interferon signal sequence IS attached to the carboxy-terminus of PRAME₄₂₅₋₄₃₃
6. Peptide antigen PRAME₄₂₅₋₄₃₃ replacing the hydrophobic portion of ES
7. Peptide antigen PRAME₄₂₅₋₄₃₃ incorporated into an artificial signal sequence - AF

Since the hydrophobicity of the fusion peptides is higher than that of the minimal peptide, a set of control fusion peptides with signal sequences situated on the carboxy-terminus of the minimal peptides were designed. Since signal sequences do not contain specific amino acid residues other than a hydrophobic region of about eight residues, modified peptides were designed by replacing this region with the hydrophobic PRAME-derived peptides.

### EXAMPLE 2

### Identification of HLA-A2.1-Restricted Immunogenic Peptides, Derived from the Antigen OFA/iLRP

OFA/iLRP-derived peptide sequences were identified that are immunogenic and can induce CTL both in healthy volunteers as well as in patients with cancer. The antigen-recognition activity of CTL is intimately linked with recognition of MHC (HLA in humans) molecules. In this invention the focus was on the HLA-A2 allele, which is the most common HLA molecule expressed by the general population in the United States. About 95% of HLA-A2+ individuals express the HLA-A2.1 subtype. For this reason, the identification of immunogenic peptides restricted by the HLA-A2.1 allele would not only serve as a proof of principle, but would also be applicable to a large portion of the patient population. The following modern methods were utilized for identification of immunogenic peptide sequences:

Manual step-wise approach to identify peptide sequences based on the known binding motifs for the HLA-A2.1 molecule. The majority of peptides bound to MHC class I molecules have a restricted size of 9±1 amino acids and require free N- and C-terminal ends. In addition to a specific size, different class I molecules appear to require a specific combination of usually two main anchor residues within their peptide ligands. In the case of the human allele HLA-A2.1, these anchor residues have been described as leucine (L) at position 2, and L or valine (V) at the C-terminal end. More recently, it was found that a “canonical” A2.1 motif could be defined as L or M (methionine) at position 2 and L, V, or I (isoleucine) at position 9. Using this approach, several 9 amino acid-long (9^{mer}) peptides have been identified within the OFA/iLRP protein sequence (Table 18):

TABLE 18

HLA-A21-restricted peptides, identified
within the OFA/iLRP sequence

| ANCHOR | ANCHOR | ANCHOR | ANCHOR |
|---|---|---|---|
| POSITION | POSITION | POSITION | POSITION |
| L at position 2 | L at position 2 | L at position 2 | M at position 2 |
| V at position 9 | L at position 9 | I at position 9 | V, L or I at |
| | | | position 9 |
| ⁷VLQMKEEDV¹⁵ | ⁵⁰NLKRTWEKL⁵⁸ | ⁵⁷KLLLAARAI⁶⁵ | NONE |
| (SEQ ID NO: 71) | (SEQ ID NO: 72) | (SEQ ID NO: 73) | |
| ⁵⁸LLLAARAIV⁶⁶ | ¹⁴⁶ALCNTDSPL¹⁵⁴ | ¹⁵³PLRYVDIAI¹⁶¹ | |
| (SEQ ID NO: 74) | (SEQ ID NO: 75) | (SEQ ID NO: 76) | |

A combination of three computer algorithms for peptide identification. The predictive algorithm, “BIMAS” ranks potential MHC binders according to the predictive half-time disassociation of peptide/MHC complexes. The second algorithm, “SYFPEITHI” ranks the peptides according to a score that takes into account the presence of primary and secondary MHC-binding anchor residues. The third algorithm, “PAProC”, predicts the proteasomal cleavages of the tumor antigens, which is a very important step in the generation of class I-restricted antigenic peptides.

The amino acid sequence of OFA/iLRP was analyzed using the “BIMAS” and the “SYFPEITHI” predictive algorithms for the existence of 9-amino acid peptides predicted to bind to HLA-A2.1. The focus was on peptides of 9 amino acids because it has been reported that HLA-A2.1 favor binding peptides of this size as compared with peptides of 8 or 10 residues. The analysis resulted in several candidate peptides for HLA-A2.1-restricted CTL epitopes. These epitopes were then analyzed with the third algorithm, “PAProC”, to verify the proteasome-mediated generation of the peptides. It was recently found that the COOH terminus of CTL epitopes requires exact cleavage by the proteasome, whereas NH2-terminal extensions of the epitope can be trimmed by putative aminopeptidase activity mainly in the ER, or in the cytosol. Therefore, the focus was on identifying peptides with the highest cleavage strength at the COOH terminus. Using all three algorithms, the search was narrowed to the following four peptides: OFA/iLRP₅₈ (LLLAARAIV) (SEQ ID NO: 74), OFA/iLRP₇ (VLQMKEEDV) (SEQ ID NO: 71), OFA/iLRP₅₇ (KLLLAARAI) (SEQ ID NO: 73), and OFA/iLRP₁₄₆ (ALCNTDSPL) (SEQ ID NO: 75). These four natural peptides were used to design synthetic vaccines with modified amino-acid residues to improve their stability, immunogenicity and antigen presentation.

### EXAMPLE 3

### Enhancing Stability, Immunogenicity, and Antigen Presentation of OFA/iLRP-Derived Synthetic Peptides

Because most attempts to treat cancer patients with TAA-derived synthetic peptides were not successful, further research aimed at enhancing the stability and immunogenicity of the peptides used for vaccination of patients with cancer is essential. The following methods were utilized.

Terminal Modifications to Inhibit Proteolytic Degradation of the OFA/iLRP Peptides:

When biologically active peptides are used clinically in their natural form, their biologic effects are often rapidly lost in vivo due to rapid elimination of the active form of the peptide. Since the skin is an enzymatically active organ, in vaccinations that utilize subcutaneous injections, peptides may be degraded by skin peptidases prior to effecting a significant immunological response. Thus, it is critical to design stable peptide formulations for vaccination of patients with cancer. The natural HLA-A2.1 restricted OFA/iLRP peptides were modified by N-terminal acetylation and/or C-terminal amidation. An example of modifications to the native HLA-A2.1 restricted peptide OFA/iLRP₅₈₋₆₆ is shown (Table 19):

TABLE 19

Terminal modifications of the
OFA/iLRP-derived peptide OFA/iLRP₅₈₋₆₆

| | | Modifications | |
|---|---|---|---|
| Peptide Name | Peptide Sequence | N-terminus | C-terminus |

| | | | |
|---|---|---|---|
| OFA/iLRP₅₈₋₆₆ | ⁵⁸LLLAARAIV⁶⁶ | — | — |
| | (SEQ ID NO: 74) | | |
| N- OFA/iLRP₅₈₋₆₆ | Ac- LLLAARAIV | Acetyl | — |
| | (SEQ ID NO: 77) | | |
| C- OFA/iLRP₅₈₋₆₆ | LLLAARAIV-amide | — | Amide |
| | (SEQ ID NO: 79) | | |
| Cap- OFA/iLRP₅₈₋₆₆ | Ac- LLLAARAIV-amide | Acetyl | Amide |
| | (SEQ ID NO: 80) | | |

Amino Acid Substitutions at HLA-A2.1 Binding Anchor Positions to Enhance MHC Class I Binding Affinity of the OFA/iLRP Peptides (Fixed Anchor Analogs):

Upon stimulation with natural peptides, tumor-reactive CTL have been induced in vitro from peripheral blood lymphocytes of some patients with cancer. However, tumor-specific CTL could only be induced in a limited number of patients, and numerous re-stimulations were required to generate anti-tumor reactivity. These findings prompted this section of the current invention aimed at enhancing the immunogenicity of peptides derived from OFA/iLRP.

As an example, the following anchor amino acid substitutions were introduced to the native HLA-A2.1 restricted peptide OFA/iLRP₅₇₋₆₅ (Table 20):

TABLE 20

Substitutions at the HLA-A2.1 binding
anchor positions

| | Peptide | |
|---|---|---|
| Peptide Name | Sequence | Substitutions |
| OFA/iLRP₅₇₋₆₅ | ⁵⁷KLLLAARAI⁶⁵ | — |
| | (SEQ ID NO:73) | |
| OFA/iLRP₅₇₋₆₅- | FLLLAARAI | F for K at position 1 |
| 1F | (SEQ ID NO:81) | |
| OFA/iLRP₅₇₋₆₅- | KLWLAARAI | W for L at position 3 |
| 3W | (SEQ ID NO:82) | |
| OFA/iLRP₅₇₋₆₅- | KLLLAARAV | V for I at position 9 |
| 9V | (SEQ ID NO:83) | |
| OFA/iLRP₅₇₋₆₅- | FLWLAARAV | all of the above |
| 1F/3W/9V | (SEQ ID NO:84) | |

Amino Acid Substitutions at NON-Anchor Positions to Enhance the T Cell Receptor Binding Affinity for the Peptide-MHC Complex (Heteroclitic Analogs):

Certain peptide analogs that carry amino acid substitutions at residues other than the main MHC anchors (heteroclitic analogs) have shown a significantly increased potency, and are surprisingly much more antigenic than wild-type peptides. These analogs may provide considerable benefit in vaccine development, as they induce stronger T cell responses than the native epitope, and have been shown to be associated with increased affinity of the epitope/MHC complex for the T cell receptor (TCR) molecule. Important advantages of the heteroclitic analogs related to their clinical application include their ability to break/overcome tolerance by reversing a state of T cell anergy and/or recruiting new T cell specificities, and the significantly smaller amounts of heteroclitic analogs that is needed for treatment.

The scheme used for selection of the single amino acid substitutions includes rank coefficient scores for PAM250, hydrophobicity, and side chain volume. The Dayhoff PAM250 score (http://prowl.rockefeller.edu/aainfo/pam250.html) is a commonly used protein alignment scoring matrix which measures the percentage of acceptable point mutations within a defined time frame.

As an example, the following NON-anchor amino acid substitutions were made to the native HLA-A2.1 restricted peptide OFA/iLRP₇₋₁₅ (Table 21):

TABLE 21
Substitutions at NON-anchor positions

| | Peptide | |
|---|---|---|
| Peptide Name | Sequence | Substitutions |
| OFA/iLRP₇₋₁₅ | ⁷VLQMKEEDV¹⁵ | — |
| | (SEQ ID NO:71) | |
| OFA/iLRP₇₋₁₅- | VLKMKEEDV | K for Q at position 3 |
| 3K | (SEQ ID NO:85) | |
| OFA/iLRP₇₋₁₅- | VLQMHEEDV | H for K at position 5 |
| 5H | (SEQ ID NO:86) | |
| OFA/iLRP₇₋₁₅- | VLQMKEPDV | P for E at position 7 |
| 7P | (SEQ ID NO:87) | |

Enhancing the Immunogenicity of the Peptides with Insertion Signal Sequences:

A variety of fusion peptides composed of natural or modified OFA/iLRP peptides and endoplasmic reticulum insertion signal sequences were designed. The following signal sequences were utilized to improve the antigen presentation: a) one from early region 3 of the adenovirus type 2—ES (MRYMILGLLALAAVCSA) (SEQ ID NO: 68), b) one from IFN-beta—IS (MTNKCLLQIALLLCFSTTALS) (SEQ ID NO: 69), and c) several artificial sequences, generated according to the structure and the distribution frequency of the amino acids in the natural signal sequences. An example of synthetic peptide constructs utilizing the epitope OFA/iLRP₅₈₋₆₆ is shown (Table 22).

TABLE 22

Synthetic peptide constructs utilizing the
epitope OFA/iLRP₅₈₋₆₆

| | |
|---|---|
| Designation | Peptide Sequence |

| | |
|---|---|
| 1. OFA/iLRP-OFA/ | ⁵⁸LLLAARAIV⁶⁶ |
| iLRP₅₈₋₆₆ | (SEQ ID NO:74) |
| 2. ES-OFA/iLRP | M R Y M I L G L L A L A A V C S |
| | A LLLAARAIV |
| | (SEQ ID NO:89) |
| 3. OFA/iLRP-ES | LLLAARAIV M R Y M I L G L L A L |
| | A A V C S A |
| | (SEQ ID NO:90) |
| 4. IS-OFA/iLRP | M T N K C L L Q I A L L L C F S |
| | T T A L S LLLAARAIV |
| | (SEQ ID NO:91) |
| 5. OFA/iLRP-IS | LLLAARAIV M T N K C L L Q I A L |
| | L L C F S T T A L S |
| | (SEQ ID NO:92) |
| 6. OFA/iLRP-IN-ES | M R LLLAARAIV A A V C S A |
| | (SEQ ID NO:93) |
| 7. OFA/iLRP-IN-AF | M A LLLAARAIV A A A A A G |
| | (SEQ ID NO:94) |

Synthetic peptide constructs:
1. Peptide antigen OFA/iLRP₅₈₋₆₆
2. Adenoviral signal sequence ES attached to the amino-terminus of OFA/iLRP₅₈₋₆₆
3. Adenoviral signal sequence ES attached to the carboxy-terminus of OFA/iLRP₅₈₋₆₆
4. Interferon signal sequence IS attached to the amino-terminus of OFA/iLRP₅₈₋₆₆
5. Interferon signal sequence IS attached to the carboxy-terminus of OFA/iLRP₅₈₋₆₆
6. Peptide antigen OFA/iLRP₅₈₋₆₆ replacing the hydrophobic portion of ES
7. Peptide antigen OFA/iLRP₅₈₋₆₆ incorporated into an artificial signal sequence - AF

Since the hydrophobicity of the fusion peptides is higher than that of the minimal peptide, a set of control fusion peptides was designed with signal sequences situated on the carboxy-terminus of the minimal peptides. Since signal sequences do not contain specific amino acid residues other than a hydrophobic region of about eight residues, modified peptides were designed by replacing this region with the hydrophobic OFA/iLRP-derived peptides.

### EXAMPLE 4

### Identification of HLA-A2.1-Restricted Immunogenic Peptides Derived from the Antigen STEAP

By the present invention, STEAP-derived peptide sequences are identified that are immunogenic and can induce CTL both in healthy volunteers as well as in patients with cancer. The antigen-recognition activity of CTL is intimately linked with recognition of MHC (HLA in humans) molecules. The invention focuses on the HLA-A2 allele, which is the most common HLA molecule expressed by the general population in the United States. About 95% of HLA-A2+ individuals express the HLA-A2.1 subtype. For this reason, the identification of immunogenic peptides restricted by the HLA-A2.1 allele would not only serve as a proof of principle, but would also be applicable to a large portion of the patient population. The following modern methods were utilized for identification of immunogenic peptide sequences:

A manual step-wise approach was used to identify peptide sequences based on the known binding motifs for the HLA-A2.1 molecule. The majority of peptides bound to MHC class I molecules have a restricted size of 9±1 amino acids and require free N- and C-terminal ends. In addition to a specific size, different class I molecules appear to require a specific combination of usually two main anchor residues within their peptide ligands. In the case of the human allele HLA-A2.1, these anchor residues have been described as leucine (L) at position 2, and L or valine (V) at the C-terminal end. More recently, it was found that a “canonical” A2.1 motif could be defined as L or M (methionine) at position 2 and L, V, or I (isoleucine) at position 9. Using this approach several 9 amino acid-long (9^{mer}) peptides have been identified within the STEAP protein sequence (Table 23):

TABLE 23
HLA-A2.1-restricted peptides, identified within the STEAP sequence

| ANCHOR | ANCHOR | ANCHOR | ANCHOR |
|---|---|---|---|
| POSITION | POSITION | POSITION | POSITION |
| L at position 2 | L at position 2 | L at position 2 | M at position 2 |
| V at position 9 | L at position 9 | I at position 9 | V, L or I at position 9 |
| ⁸⁶FLYTLLREV⁹⁴ | ⁸³SLTFLYTLL⁹¹ | ⁷²HLPIKIAAI⁸⁰ | ³⁶SMLKRPVLL⁴⁴ |
| (SEQ ID NO:96) | (SEQ ID NO:97) | (SEQ ID NO:98) | (SEQ ID NO:99) |
| ¹⁶⁵GLLSFFFAV¹⁷³ | ⁹⁰LLREVIHPL⁹⁸ | ¹²⁷ALVYLPGVI¹³⁵ | ²⁹¹FMIAVFLPI²⁹⁹ |
| (SEQ ID NO:100) | (SEQ ID NO:101) | (SEQ ID NO:102) | (SEQ ID NO:103) |
| ¹⁹²LLNWAYQQV²⁰⁰ | ¹¹⁷VLPMVSITL¹²⁵ | ¹³⁰YLPGVIAAI¹³⁸ | |
| (SEQ ID NO:104) | (SEQ ID NO:105) | (SEQ ID NO:106) | |
| | ¹⁵⁸MLTRKQFGL¹⁶⁶ | ²²¹SLGIVGLAI²²⁹ | |
| | (SEQ ID NO:107) | (SEQ ID NO:108) | |
| | ¹⁶⁶LLSFFFAVL¹⁷⁴ | ²⁶³LLGTIHALI²⁷¹ | |
| | (SEQ ID NO:109) | (SEQ ID NO:110) | |
| | ²⁵⁶KLGIVSLLL²⁶⁴ | ³⁰⁹FLPCLRKKI³¹⁷ | |
| | (SEQ ID NO:111) | (SEQ ID NO:112) | |
| | ²⁶²LLLGTIHAL²⁷⁰ | ³¹²CLRKKILKI³²⁰ | |
| | (SEQ ID NO:113) | (SEQ ID NO:114) | |

A combination of three computer algorithms was utilized for peptide identification. The predictive algorithm, “BIMAS” ranks potential MHC binders according to the predictive half-time disassociation of peptide/MHC complexes. The second algorithm, “SYFPEITHI” ranks the peptides according to a score that takes into account the presence of primary and secondary MHC-binding anchor residues. The third algorithm, “PAProC”, predicts the proteasomal cleavages of the tumor antigens, which is a very important step in the generation of class I-restricted antigenic peptides.

The amino acid sequence of STEAP was analyzed by using the “BIMAS” and the “SYFPEITHI” predictive algorithms for the existence of 9-amino acid peptides predicted to bind to HLA-A2.1. Peptides of 9 amino acids were the focus because it has been reported that HLA-A2.1 favor binding peptides of this size as compared with peptides of 8 or 10 residues. The analysis resulted in several candidate peptides for HLA-A2.1-restricted CTL epitopes. These epitopes were then analyzed with the third algorithm, “PAProC”, to verify the proteasome-mediated generation of the peptides. It was recently found that the COOH terminus of CTL epitopes requires exact cleavage by the proteasome, whereas NH2-terminal extensions of the epitope can be trimmed by putative aminopeptidase activity mainly in the ER, or in the cytosol. Therefore, the focus was on identifying peptides with the highest cleavage strength at the COOH terminus. Using all three algorithms, the search was narrowed to the following five peptides: ¹³⁰YLPGVIAAI¹³⁸ (SEQ ID NO: 106), ¹⁶⁵GLLSFFFAV¹⁷³ (SEQ ID NO: 100), ¹⁶⁶LLSFFFAVL¹⁷⁴ (SEQ ID NO: 109), ¹⁹²LLNWAYQQV²⁰⁰ (SEQ ID NO: 104) and ³⁰²LIFKSILFL³¹⁰ (SEQ ID NO: 115). These five natural peptides were the starting point, however, more peptides were designed with modification of amino-acid residues to improve the stability, immunogenicity and antigen presentation of the peptides.

### EXAMPLE 5

### Enhancing the Stability, Immunogenicity, and Antigen Presentation of STEAP-Derived Synthetic Peptides

Terminal Modifications to Inhibit Proteolytic Degradation of the STEAP Peptides:

When biologically active peptides are used clinically in their natural form, their biologic effects are often rapidly lost in vivo due to rapid elimination of the active form of the peptide. Since the skin is an enzymatically active organ, in vaccinations that utilize subcutaneous injections, peptides may be degraded by skin peptidases prior to effecting a significant immunological response. Thus, it is critical to design stable peptide formulations for vaccination of patients with cancer. The natural HLA-A2.1 restricted STEAP peptides were modified by N-terminal acetylation and/or C-terminal amidation. An example of modifications to the native HLA-A2.1 restricted peptide STEAP₁₃₀₋₁₃₈ is shown (Table 24):

TABLE 24

Terminal modifications of the STEAP-derived
peptide STEAP₁₃₀₋₁₃₈

| | | Modifications | |
|---|---|---|---|
| Peptide Name | Peptide Sequence | N-Terminus | C-Terminus |

| | | | |
|---|---|---|---|
| STEAP₁₃₀₋₁₃₈ | ¹³⁰YLPGVIAAI¹³⁸ | — | — |
| | (SEQ ID NO:106) | | |
| N-STEAP₁₃₀₋₁₃₈ | Ac-YLPGVIAAI | Acetyl | — |
| | (SEQ ID NO:116) | | |
| C-STEAP₁₃₀₋₁₃₈ | YLPGVIAAI-amide | — | Amide |
| | (SEQ ID NO:117) | | |
| Cap-STEAP₁₃₀₋₁₃₈ | Ac-YLPGVIAAI-amide | Acetyl | Amide |
| | (SEQ ID NO:118) | | |

Amino Acid Substitutions at HLA-A2.1 Binding Anchor Positions to Enhance MHC Class I Binding Affinity of the STEAP Peptides (Fixed Anchor Analogs):

Upon stimulation with natural peptides, tumor-reactive CTL have been induced in vitro from peripheral blood lymphocytes of some patients with cancer. However, tumor-specific CTL could only be induced in a limited number of patients, and numerous restimulations were required to generate anti-tumor reactivity. These findings prompted this section of the current invention aimed at enhancing the immunogenicity of peptides derived from STEAP.

As an example, the following anchor amino acid substitutions were made to the native HLA-A2.1 restricted peptide STEAP₁₃₀₋₁₃₈ (Table 25):

TABLE 25

Substitutions at the HLA-A2.1 binding
anchor positions

| | Peptide | |
|---|---|---|
| Peptide Name | Sequence | Substitutions |
| STEAP₁₃₀₋₁₃₈ | ¹³⁰YLPGVIAAI¹³⁸ | — |
| | (SEQ ID NO:106) | |
| STEAP₁₃₀₋₁₃₈- | FLPGVIAAI | F for Y at position 1 |
| IF | (SEQ ID NO:119) | |
| STEAP₁₃₀₋₁₃₈- | YLWGVIAAI | W for P at position 3 |
| 3W | (SEQ ID NO:120) | |
| STEAP₁₃₀₋₁₃₈- | YLPGVIAAV | V for I at position 9 |
| 9V | (SEQ ID NO:121) | |
| STEAP₁₃₀₋₁₃₈- | FLWGVIAAV | all of the above |
| 1F/3W/9V | (SEQ ID NO:122) | |

Amino Acid Substitutions at NON-Anchor Positions to Enhance the T Cell Receptor Binding Affinity for the Peptide-MHC Complex (Heteroclitic Analogs):

Certain peptide analogs that carry amino acid substitutions at residues other than the main MHC anchors (heteroclitic analogs) have shown a significantly increased potency, and are surprisingly much more antigenic than wild-type peptides. These analogs may provide considerable benefit in vaccine development, as they induce stronger T cell responses than the native epitope, and have been shown to be associated with increased affinity of the epitope/MHC complex for the T cell receptor (TCR) molecule. Important advantages of the heteroclitic analogs related to their clinical application include their ability to break/overcome tolerance by reversing a state of T cell anergy and/or recruiting new T cell specificities, and the significantly smaller amounts of heteroclitic analogs that is needed for treatment.

The scheme that used for selection of the single amino acid substitutions includes rank coefficient scores for PAM250, hydrophobicity, and side chain volume. The Dayhoff PAM250 score (hyper text transfer protocol address prowl.rockefeller.edu/aainfo/pam250.htm) is a commonly used protein alignment scoring matrix which measures the percentage of acceptable point mutations within a defined time frame.

As an example, the following NON-anchor amino acid substitutions were made to the native HLA-A2.1 restricted peptide STEAP₁₃₀₋₁₃₈ (Table 26):

TABLE 26
Substitutions at NON-anchor positions

| | Peptide | |
|---|---|---|
| Peptide Name | Sequence | Substitutions |
| STEAP₁₃₀₋₁₃₈ | ¹³⁰YLPGVIAAI¹³⁸ | — |
| | (SEQ ID NO:106) | |
| STEAP₁₃₀₋₁₃₈- | YLKGVIAAI | K for P at position 3 |
| 3K | (SEQ ID NO:123) | |
| STEAP₁₃₀₋₁₃₈- | YLPGHIAAI | H for V at position 5 |
| 5H | (SEQ ID NO:124) | |
| STEAP₁₃₀₋₁₃₈- | YLPGVIPAI | P for A at position 7 |
| 7P | (SEQ ID NO:125) | |

Enhancing the Immunogenicity of the Peptides with Insertion Signal Sequences:

The transport of antigenic peptides from the cytosol to the endoplasmic reticulum (ER) is a limiting step in processing and presentation of class I-restricted antigens. Bypassing this step by direct targeting of the antigen to the ER can result in more effective generation of CTL. This could amount to a more potent CTL induction and anti-tumor immunity against prostate cancer and breast cancer. A variety of fusion peptides composed of natural or modified STEAP peptides and endoplasmic reticulum insertion signal sequences were designed. The following signal sequences were utilized to improve the antigen presentation: a) one from early region 3 of the adenovirus type 2—ES (MRYMILGLLALAAVCSA) (SEQ ID NO:68), b) one from IFN-beta—IS (MTNKCLLQIALLLCFSTTALS) (SEQ ID NO: 69), and c) several artificial sequences, generated according to the structure and the distribution frequency of the amino acids in the natural signal sequences. An example of synthetic peptide constructs utilizing the epitope STEAP₁₃₀₋₁₃₈ is shown (Table 27).

TABLE 27

Synthetic peptide constructs utilizing
the epitope STEAP₁₃₀₋₁₃₈

| | |
|---|---|
| Designation | Peptide Sequence |

| | |
|---|---|
| 1. STEAP-STEAP₁₃₀₋₁₃₈ | YLPGVIAAI |
| | (SEQ ID NO:106) |
| 2. ES-STEAP | M R Y M I L G L L A L A A V C |
| | S A YLPGVIAAI |
| | (SEQ ID NO:126) |
| 3. STEAP-ES | YLPGVIAAI M R Y M I L G L L A |
| | L A A V C S A |
| | (SEQ ID NO:127) |
| 4. IS-STEAP | M T N K C L L Q I A L L L C F |
| | S T T A L S YLPGVIAAI |
| | (SEQ ID NO:128) |
| 5. STEAP-IS | YLPGVIAAI M T N K C L L Q I A |
| | L L L C F S T T A L S |
| | (SEQ ID NO:129) |
| 6. STEAP-IN-ES | M R YLPGVIAAI A A V C S A |
| | (SEQ ID NO:130) |
| 7. STEAP-IN-AF | M A YLPGVIAAI A A A A A G |
| | (SEQ ID NO:131) |

Synthetic peptide constructs:
1. Peptide antigen STEAP₁₃₀₋₁₃₈
2. Adenoviral signal sequence ES attached to the amino-terminus of STEAP₁₃₀₋₁₃₈
3. Adenoviral signal sequence ES attached to the carboxy-terminus of STEAP₁₃₀₋₁₃₈
4. Interferon signal sequence IS attached to the amino-terminus of STEAP ₁₃₀₋₁₃₈
5. Interferon signal sequence IS attached to the carboxy-terminus of STEAP₁₃₀₋₁₃₈
6. Peptide antigen STEAP₁₃₀₋₁₃₈ replacing the hydrophobic portion of ES
7. Peptide antigen STEAP₁₃₀₋₁₃₈ incorporated into an artificial signal sequence - AF

Since the hydrophobicity of the fusion peptides is higher than that of the minimal peptide, a set of control fusion peptides were designed with signal sequences situated on the carboxy-terminus of the minimal peptides. Since signal sequences do not contain specific amino acid residues other than a hydrophobic region of about eight residues, modified peptides were designed by replacing this region with the hydrophobic STEAP-derived peptides.

### EXAMPLE 6

### Induction of Peptide-Specific (CTL) In Vitro

This example tested whether the STEAP-derived natural and modified peptides can induce CTL by in vitro immunization of blood cells from healthy donors and from patients with breast cancer with these peptides. Peripheral blood mononuclear cells (PBMC) were isolated from HLA-A2.1+ healthy volunteers and cancer patients by centrifugation on Ficoll-Hypaque gradients. PBMC were then plated in 24-well plates at 5×10⁵ cells/ml/well in RPMI-1640 supplemented with 10% human AB⁺ serum, L-glutamine and antibiotics (CM). Autologous PBMC were pulsed with 10 μg/ml STEAP peptide for 3 hours at 37° C. These PBMC (stimulators) were then irradiated at 3000 rads, washed once, and added to the responder cells at responder:stimulator ratios ranging between 1:1 and 1:4. The next day, 12 IU/ml IL-2 and 30 IU/ml IL-7 were added to the cultures. Lymphocytes were then re-stimulated weekly with peptide-pulsed adherent cells as follows: previously frozen autologous PBMC were thawed, washed, re-suspended at 4×10⁶ cells/ml in CM containing 10 μg/ml peptide, and plated in 24-well plates at 1 ml/well. Plates were incubated for 3 hours at 37° C. and the non-adherent cells were removed by a gentle wash with PBS. Fresh complete media containing 10 μg/ml peptide were added to the cells, and the plates were incubated again for 3 hours at 37° C. Responder cells were harvested, washed once and added to the peptide-pulsed adherent cells at a concentration of 5×10⁵ cells/ml (2 ml/well) in complete media. IL-2 and IL-7 were added to the cultures on the next day. The activity of these CTL was tested by a LDH-release cytotoxicity assays (Cytotox96 kit, Promega) after at least two rounds of peptide stimulation. K562 cells transfected with HLA-A2.1+ were pulsed with the STEAP peptides, and used as targets.

Most of the tested STEAP-derived peptides were able to induce peptide-specific CTL. The natural peptides STEAP₁₃₀₋₁₃₈, STEAP₁₆₆₋₁₇₄, and STEAP₁₉₂₋₂₀₀, as well as the modified peptides STEAP_{130-138-1F}, STEAP_{130-138-3W}, STEAP_{130-138-9V}, and STEAP_{130-138-1F/3W/9V} induced potent peptide-specific CTL (Table 28):

TABLE 28
Specific recognition of peptide-pulsed
target cells by STEAP-induced CTL

Percent LDH released from^{a}:

| | | K562-A2 pulsed with |
|---|---|---|
| CTL specific for: | K562-A2 | peptide^{b} |
| STEAP₁₃₀₋₁₃₈ | 1 | 63 |
| STEAP₁₃₀₋₁₃₈ - 1F | 4 | 75 |
| STEAP₁₃₀₋₁₃₈ - 3W | 4 | 63 |
| STEAP₁₃₀₋₁₃₈ - 9V | 1 | 53 |
| STEAP₁₃₀₋₁₃₈ - 1F/3W/9V | 1 | 74 |
| STEAP₁₆₆₋₁₇₄ | 3 | 43 |
| STEAP₁₉₂₋₂₀₀ | 8 | 56 |

^{a}Cytotoxicity was evaluated in a 4-hour LDH-release assay
^{b}K562-A2 cells were pulsed with corresponding STEAP peptide for 2 hours at 37° C. and used as targets

The peptide STEAP₁₉₂₋₂₀₀ induced peptide-specific CTL in three out of three patients with prostate cancer.

These findings suggest that most STEAP-derived peptides tested so far are immunogenic, implying that precursor CTL for STEAP are present in the peripheral adult repertoire.

### EXAMPLE 7

### Testing the Ability of the STEAP-Specific CTL to Recognize and Kill Prostate Cancer Cells in a Class I-Restricted and Antigen-Dependent Fashion

It was tested whether the STEAP-derived peptides can induce potent CTL capable of recognizing and killing prostate cancer cells in vitro. CTL lines selected for their ability to lyse peptide-pulsed target cells were used as effectors in LDH-release cytotoxicity assays against the cancer cell lines. The HLA-A2+ cancer cell line LnCAP was used, with the HLA-A2−negative cancer cell line DU145 as a control (Table 29).

TABLE 29
Specific recognition of prostate cancer cell lines
by CTL reactive against the STEAP-derived peptides

| | Percent LDH released from^{a}: | |
|---|---|---|
| CTL specific for: | LnCap (HLA-A2+) | DU145 (HLA-A2−) |
| STEAP₁₃₀₋₁₃₈ | 53 | −1 |
| STEAP₁₃₀₋₁₃₈ - 1F | 73 | −1 |
| STEAP₁₃₀₋₁₃₈ - 3W | 69 | −2 |
| STEAP₁₃₀₋₁₃₈ - 9V | 68 | −2 |
| STEAP₁₃₀₋₁₃₈ - 1F/3W/9V | 84 | 1 |
| STEAP₁₆₆₋₁₇₄ | 65 | 4 |
| STEAP₁₉₂₋₂₀₀ | 43 | 18 |

^{a}Cytotoxicity was evaluated in a 4-hour LDH-release assay

To determine if the lysis of the target cells is HLA-A2 restricted, blocking experiments were performed using the anti-HLA-A2 antibody BB7.2, which was added to the cancer cells prior to the addition of CTL. As an additional control the anti-HLA class II antibody IVA12 was used. With these experiments, it was confirmed that the STEAP-specific CTL can recognize and kill target cells in a class I-restricted fashion (Table 30).

TABLE 30
Class I-restricted specific recognition of target cells
by CTL reactive against the STEAP-derived peptides

| | Percent LDH released from^{a} | | | |
|---|---|---|---|---|
| | | K562-A2 | | |
| CTL specific for: | K562-A2 | pulsed^{b} | +BB7.2 | +IVA12^{c} |
| STEAP₁₃₀₋₁₃₈ | 1 | 63 | 2 | 37 |
| STEAP₁₃₀₋₁₃₈ -1F | 4 | 75 | 2 | 69 |
| STEAP₁₃₀₋₁₃₈ -3W | 4 | 63 | 5 | 64 |
| STEAP₁₃₀₋₁₃₈ -9V | 1 | 53 | 1 | 47 |
| STEAP₁₃₀₋₁₃₈ -1F/3W/9V | 1 | 74 | 1 | 75 |
| STEAP₁₆₆₋₁₇₄ | 3 | 43 | 11 | 47 |
| STEAP₁₉₂₋₂₀₀ | 8 | 56 | 7 | 30 |

^{a}Cytotoxicity was evaluated in a 4-hour LDH-release assay
^{b}K562-A2 cells were pulsed with corresponding STEAP peptide for 2 hours at 37° C. and used as targets
^{c}The blocking antibodies BB7.2 and IVA12 were added to the peptide-pulsed target cells before the 4-hour LDH-release assay

Collectively, these data indicate that the STEAP-derived peptides are naturally processed in prostate cancer cell lines in a class I-restricted fashion.

### EXAMPLE 8

### Identification of HLA-A2.1-Restricted Immunogenic Peptides Derived from the Antigen SURVIVIN

By the present invention, SURVIVIN-derived peptide sequences are identified that are immunogenic and can induce CTL, both in healthy volunteers as well as in patients with cancer. The antigen-recognition activity of CTL is intimately linked with recognition of MHC (HLA in humans) molecules. The invention focuses on the HLA-A2 allele, which is the most common HLA molecule expressed by the general population in the United States. About 95% of HLA-A2+ individuals express the HLA-A2.1 subtype. For this reason, the identification of immunogenic peptides restricted by the HLA-A2.1 allele would not only serve as a proof of principle, but would also be applicable to a large portion of the patient population. The following modern methods were utilized for identification of immunogenic peptide sequences.

A manual step-wise approach was used to identify peptide sequences based on the known binding motifs for the HLA-A2.1 molecule. The majority of peptides bound to MHC class I molecules have a restricted size of 9±1 amino acids and require free N- and C-terminal ends. In addition to a specific size, different class I molecules appear to require a specific combination of usually two main anchor residues within their peptide ligands. In the case of the human allele HLA-A2.1, these anchor residues have been described as leucine (L) at position 2, and L or valine (V) at the C-terminal end. More recently, it was found that a “canonical” A2.1 motif could be defined as L or M (methionine) at position 2 and L, V, or I (isoleucine) at position 9. Using this approach several 9 amino acid-long (9^{mer}) peptides have been identified within the SURVIVIN protein sequence (Table 31):

TABLE 31

HLA-A2.1-restricted peptides, identified
within the SURVIVIN sequence

| | |
|---|---|
| HLA-A*0201 nonamers | HLA-A*0201 decamers |

| | |
|---|---|
| ²⁰STFKNWPFL²⁸ | ⁵TLPPAWQPFL¹⁴ |
| (SEQ ID NO:160) | (SEQ ID NO:161) |
| ²³KNWPFLEGC³¹ | ¹²²KEFEETAKKV¹³¹ |
| (SEQ ID NO:162) | (SEQ ID NO:163) |
| ⁹⁶LTLGEFLKL¹⁰⁴ | ⁹⁵ELTLGEFLKL¹⁰⁴ |
| (SEQ ID NO:164) | (SEQ ID NO:165) |
| ⁶LPPAWQPFL¹⁴ | ¹⁹ISTFKNWPFL²⁸ |
| (SEQ ID NO:166) | (SEQ ID NO:167) |
| ³³CTPERMAEA⁴¹ | ⁴⁸TENEPDLAQC⁵⁷ |
| (SEQ ID NO:168) | (SEQ ID NO:169) |
| ⁴⁶CPTENEPDL⁵⁴ | ⁹³FEELTLGEFL¹⁰² |
| (SEQ ID NO:170) | (SEQ ID NO:171) |
| ¹³⁰KVRRAIEQL¹³⁸ | ⁸⁷LSVKKQFEEL⁹⁶ |
| (SEQ ID NO:172) | (SEQ ID NO:173) |
| ³⁷RMAEAGFIH⁴⁵ | ¹²⁹KKVRRAIEQL¹³⁸ |
| (SEQ ID NO:174) | (SEQ ID NO:175) |
| ⁸⁸SVKKQFEEL⁹⁶ | ¹³FLKDHRISTF²² |
| (SEQ ID NO:176) | (SEQ ID NO:177) |
| | ³²ACTPERMAEA⁴¹ |
| | (SEQ ID NO:178) |

A combination of three computer algorithms was utilized for peptide identification. The predictive algorithm, “BIMAS” ranks potential MHC binders according to the predictive half-time disassociation of peptide/MHC complexes. The second algorithm, “SYFPEITHI” ranks the peptides according to a score that takes into account the presence of primary and secondary MHC-binding anchor residues. The third algorithm, “PAProC”, predicts the proteasomal cleavages of the tumor antigens, which is a very important step in the generation of class I-restricted antigenic peptides.

The amino acid sequence of SURVIVIN was analyzed by using the “BIMAS” and the “SYFPEITHI” predictive algorithms for the existence of 9-amino acid peptides predicted to bind to HLA-A2.1. Peptides of 9 amino acids were the focus because it has been reported that HLA-A2.1 favor binding peptides of this size as compared with peptides of 8 or 10 residues. The analysis resulted in several candidate peptides for HLA-A2.1-restricted CTL epitopes. These epitopes were then analyzed with the third algorithm, “PAProC”, to verify the proteasome-mediated generation of the peptides. It was recently found that the COOH terminus of CTL epitopes requires exact cleavage by the proteasome, whereas NH2-terminal extensions of the epitope can be trimmed by putative aminopeptidase activity mainly in the ER, or in the cytosol. Therefore, the focus was on identifying peptides with the highest cleavage strength at the COOH terminus.

### EXAMPLE 9

### Enhancing the Stability, Immunogenicity, and Antigen Presentation of SURVIVIN-Derived Synthetic Peptides

Terminal Modifications to Inhibit Proteolytic Degradation of the SURVIVIN Peptides:

When biologically active peptides are used clinically in their natural form, their biologic effects are often rapidly lost in vivo due to rapid elimination of the active form of the peptide. Since the skin is an enzymatically active organ, in vaccinations that utilize subcutaneous injections, peptides may be degraded by skin peptidases prior to effecting a significant immunological response. Thus, it is critical to design stable peptide formulations for vaccination of patients with cancer. The natural HLA-A2.1 restricted SURVIVIN peptides were modified by N-terminal acetylation and/or C-terminal amidation. An example of modifications to the native HLA-A2.1 restricted peptide survivin₂₀₋₂₈ is shown (Table 32):

TABLE 32

Terminal modifications of the survivin-derived
peptide survivin₂₀₋₂₈

| | | Modifications | |
|---|---|---|---|
| Peptide Name | Peptide Sequence | N-Terminus | C-Terminus |

| | | | |
|---|---|---|---|
| survivin₂₀₋₂₈ | ²⁰STFKNWPFL²⁸ | — | — |
| | (SEQ ID NO:160) | | |
| N- survivin₂₀₋₂₈ | Ac-STFKNWPFL | Acetyl | — |
| | (SEQ ID NO:179) | | |
| C survivin₂₀₋₂₈ | STFKNWPFL-amide | — | Amide |
| | (SEQ ID NO:180) | | |
| Cap-survivin₂₀₋₂₈ | Ac-STFKNWPFL-amide | Acetyl | Amide |
| | (SEQ ID NO:181) | | |

Amino Acid Substitutions at HLA-A2.1 Binding Anchor Positions to Enhance MHC Class I Binding Affinity of the SURVIVIN Peptides (Fixed Anchor Analogs):

Upon stimulation with natural peptides, tumor-reactive CTL have been induced in vitro from peripheral blood lymphocytes of some patients with cancer. However, tumor-specific CTL could only be induced in a limited number of patients, and numerous restimulations were required to generate anti-tumor reactivity. These findings prompted this section of the current invention aimed at enhancing the immunogenicity of peptides derived from SURVIVIN.

As an example, the following anchor amino acid substitutions were made to the native HLA-A2.1 restricted peptide survivin₂₀₋₂₈ (Table 33):

TABLE 33

Substitutions at the HLA-A*0201 binding
anchor positions

| | | |
|---|---|---|
| Peptide Name | Peptide Sequence | Substitutions |

| | | |
|---|---|---|
| survivin₂₀₋₂₈g | ²⁰STFKNWPFL²⁸ | — |
| | (SEQ ID NO:160) | |
| survivin₂₀₋₂₈- | SLFKNWPFL | L for T at P2 |
| 2L | (SEQ ID NO:182) | |
| survivin₂₀₋₂₈- | ATFKNWPFL | A for S at P1 |
| 1A | (SEQ ID NO:183) | |
| survivin₂₀₋₂₈- | ALFKNWPFL | both |
| 2L/1A | (SEQ ID NO:184) | substitutions |

Amino Acid Substitutions at NON-Anchor Positions to Enhance the T Cell Receptor Binding Affinity for the Peptide-MHC Complex (Heteroclitic Analogs):

Certain peptide analogs that carry amino acid substitutions at residues other than the main MHC anchors (heteroclitic analogs) have shown a significantly increased potency, and are surprisingly much more antigenic than wild-type peptides. These analogs may provide considerable benefit in vaccine development, as they induce stronger T cell responses than the native epitope, and have been shown to be associated with increased affinity of the epitope/MHC complex for the T cell receptor (TCR) molecule. Important advantages of the heteroclitic analogs related to their clinical application include their ability to break/overcome tolerance by reversing a state of T cell anergy and/or recruiting new T cell specificities, and the significantly smaller amounts of heteroclitic analogs that is needed for treatment.

The scheme that used for selection of the single amino acid substitutions includes rank coefficient scores for PAM250, hydrophobicity, and side chain volume. The Dayhoff PAM250 score (hyper text transfer protocol address prowl.rockefeller.edu/aainfo/pam250.htm) is a commonly used protein alignment scoring matrix which measures the percentage of acceptable point mutations within a defined time frame.

As an example, the following NON-anchor amino acid substitutions were made to the native HLA-A2.1 restricted peptide survivin₂₀₋₂₈ (Table 34):

TABLE 34
Substitutions at NON-anchor positions

| | | |
|---|---|---|
| Peptide Name | Peptide Sequence | Substitutions |

| | | |
|---|---|---|
| survivin₂₀₋₂₈ | ²⁰STFKNWPFL²⁸ | — |
| | (SEQ ID NO:160) | |
| survivin₂₀₋₂₈ | STKKNWPFL | K for F at P3 |
| 3K | (SEQ ID NO:185) | |
| survivin₂₀₋₂₈ | STFKHWPFL | H for N at P5 |
| 5H | (SEQ ID NO:186) | |

Enhancing the Immunogenicity of the Peptides with Insertion Signal Sequences

The transport of antigenic peptides from the cytosol to the endoplasmic reticulum (ER) is a limiting step in processing and presentation of class I-restricted antigens. Bypassing this step by direct targeting of the antigen to the ER can result in more effective generation of CTL. This could amount to a more potent CTL induction and anti-tumor immunity against prostate cancer and breast cancer. A variety of fusion peptides composed of natural or modified STEAP peptides and endoplasmic reticulum insertion signal sequences were designed. The following signal sequences were utilized to improve the antigen presentation: a) one from early region 3 of the adenovirus type 2—ES (MRYMILGLLALAAVCSA) (SEQ ID NO:68), b) one from IFN-beta—IS (MTNKCLLQIALLLCFSTTALS) (SEQ ID NO: 69), and c) several artificial sequences, generated according to the structure and the distribution frequency of the amino acids in the natural signal sequences. An example of synthetic peptide constructs utilizing the epitope survivin₂₀₋₂₈ is shown (Table 35):

TABLE 35

Synthetic peptide constructs utilizing the
epitope survivin₂₀₋₂₈

| | |
|---|---|
| DESIGNATION | PEPTIDE SEQUENCE |

| | |
|---|---|
| survivin₂₀₋₂₈ | STFKNWPFL |
| | (SEQ ID NO:160) |
| ES-survivin₂₀₋₂₈ | M R Y M L L G L L A L A A V C S |
| | A STFKNWPFL |
| | (SEQ ID NO:187) |
| survivin₂₀₋₂₈-ES | STFKNWPFL M R Y M I L G L L A L |
| | A A V C S A |
| | (SEQ ID NO:188) |
| IS-survivin₂₀₋₂₈ | M T N K C L L Q I A L L L C F S |
| | T T A L S STFKNWPFL |
| | (SEQ ID NO:189) |
| survivin₂₀₋₂₈-IS | STFKNWPFL M T N K C L L Q I A L |
| | L L C F S T T A L S |
| | (SEQ ID NO:190) |
| survivin₂₀₋₂₈-IN-ES | M R STFKNWPFL A A V C S A |
| | (SEQ ID NO:191) |
| survivin₂₀₋₂₈-IN-AF | M A STFKNWPFL A A A A A G |
| | (SEQ ID NO:192) |

Synthetic peptide constructs:
1. Peptide antigen survivin
2. Adenoviral signal sequence ES attached to the amino-terminus of survivin₂₀₋₂₈
3. Adenoviral signal sequence ES attached to the carboxy-terminus of survivin₂₀₋₂₈
4. Interferon signal sequence IS attached to the amino-terminus of survivin₂₀₋₂₈
5. Interferon signal sequence IS attached to the carboxy-terminus of survivin₂₀₋₂₈
6. Peptide antigen survivin₂₀₋₂₈ replacing the hydrophobic portion of ES
7. Peptide antigen survivin₂₀₋₂₈ incorporated into an artificial signal sequence - AF

Since the hydrophobicity of the fusion peptides is higher than that of the minimal peptide, a set of control fusion peptides were designed with signal sequences situated on the carboxy-terminus of the minimal peptides. Since signal sequences do not contain specific amino acid residues other than a hydrophobic region of about eight residues, modified peptides were designed by replacing this region with the hydrophobic SURVIVIN-derived peptides.

### EXAMPLE 10

### Testing the Effectiveness of the Fusion Peptides with T2 Cells

To probe class I presentation of cells loaded with the fusion peptides and their counterpart minimal peptides CTL recognizing the HER2/neu-derived peptides were generated. In vitro peripheral blood mononuclear cells (PBMCs) were immunized from healthy donors with these peptides in the presence of interleukin 2 and interleukin 7 using the following technique:

PBMCs were separated by centrifugation on Ficoll-Hypaque gradients and plated in 24-well plates at 5×10⁵ cells/ml per well in RPMI medium 1640 supplemented with 10% human AB⁺ serum, L-glutamine, and antibiotics. Autologous PBMC (stimulators) were pulsed with the HER2/neu synthetic peptides (10 μg/ml) for 3 h at 37° C. Cells were then irradiated at 3,000 rads, washed once, and added to the responder cells at a responder to stimulator ratio ranging between 1:1 and 1:4. The next day, 12 units/ml IL-2 (Chiron) and 30 units/ml IL-7 (R & D Systems) were added to the cultures. Lymphocytes were re-stimulated weekly with peptide-pulsed autologous adherent cells as follows: First, autologous PBMC were incubated with HER2/neu peptide (10 μg/ml) for 3 h at 37° C. Nonadherent cells were then removed by a gentle wash and the adherent cells were incubated with fresh medium containing the HER2/neu peptide (10 μg/ml) for an additional 3 h at 37° C. Second, responder cells from a previous stimulation cycle were harvested, washed, and added to the peptide-pulsed adherent cells at a concentration of 5×10⁵ cells/ml (2 ml/well) in medium without peptide. Recombinant IL-2 and IL-7 were added to the cultures the next day.

The induction of CTL in human PBMC was monitored in a conventional ⁵¹Cr-labeling release assay. Briefly, peptide-pulsed TAP⁻/HLA-A2.1⁺ human T2 cells were incubated with 10 μg of HER2/neu peptides or the MART-1 control peptide for 90 min during labeling with ⁵¹Cr. After washing, the target cells were added to serially diluted effectors in 96-well microplates. After a 6-h incubation at 37° C., supernatants were harvested and counted in a gamma counter. Results are expressed as the percentage of specific lysis and determined as follows: [(experimental cpm−spontaneous cpm)/(maximum cpm−spontaneous cpm)]×100. (Table 36).

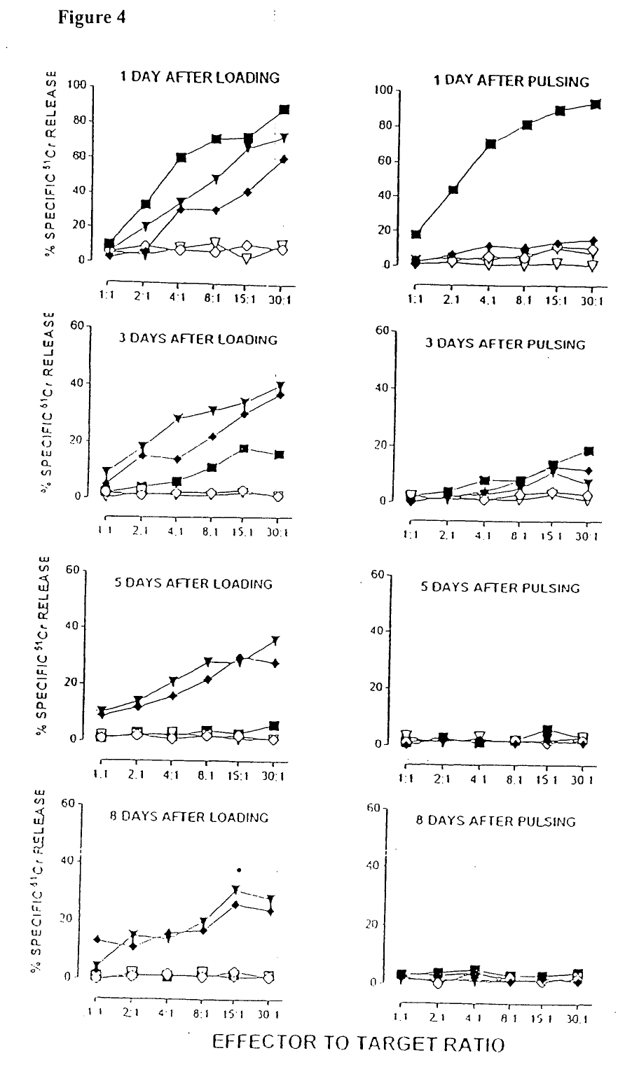

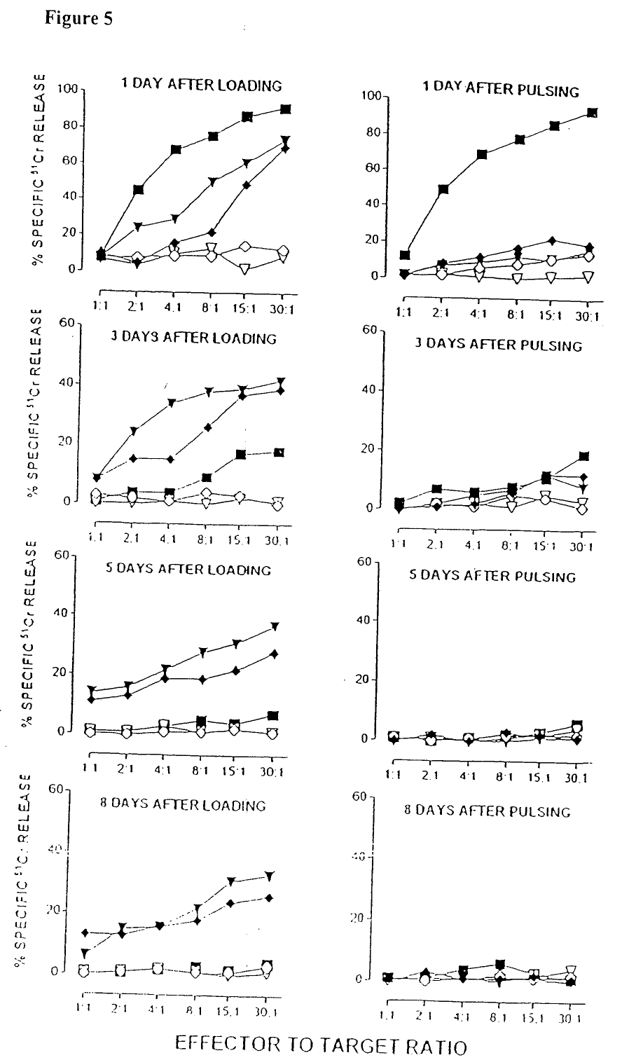

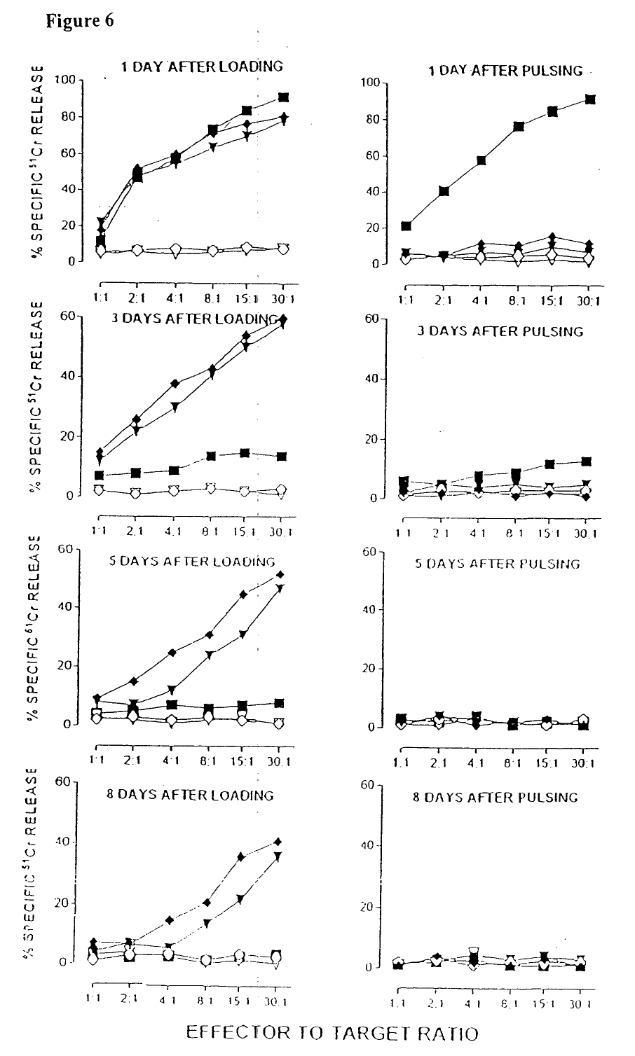

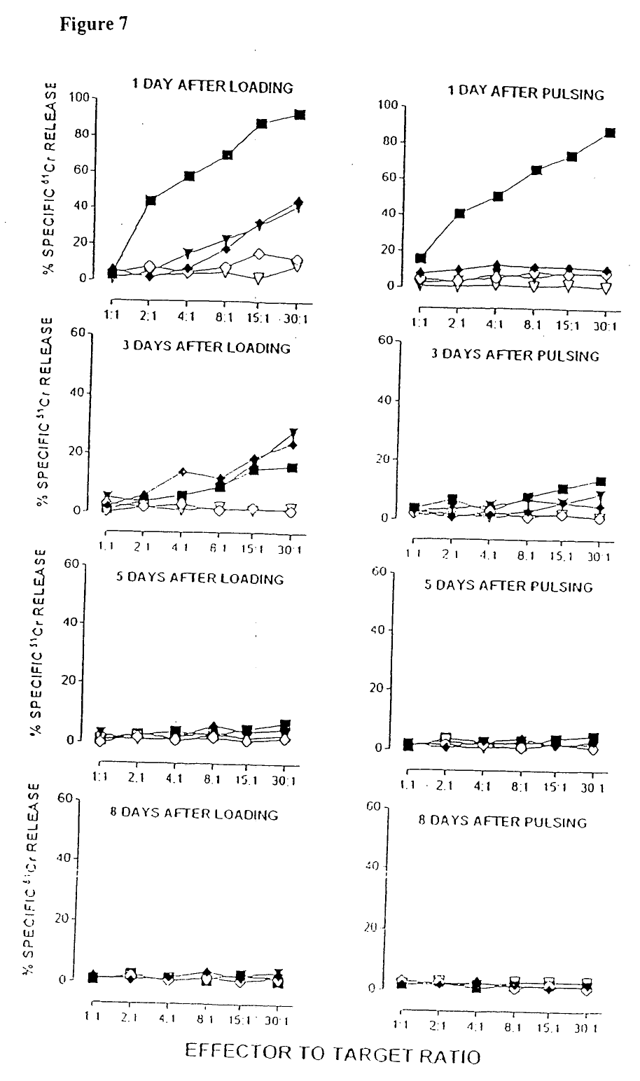

Peptide-loaded or pulsed T2 cells were tested for their ability to present HER2/neu peptides at different periods of time after loading or pulsing. T2 cells loaded with most of the constructs composed of signal sequence at the amino-terminus of HER2/neu peptides were recognized by CTL up to eight days after loading (FIGS. 4-6, left column). In contrast, constructs with carboxy-terminal position of the signal sequence were not efficient, even when ⁵¹Cr-release assays were performed immediately after loading. This recognition was not due to surface binding of these constructs since pulsing of T2 cells with any of the constructs was not efficient (FIGS. 4-7, right column). Loading or pulsing with the minimal HER2/neu peptides resulted in a significant recognition and lysis of T2 cells for only one day after loading or pulsing, followed by a rapid decrease of recognition on day 3 and complete lack of recognition on days 5 and 8 after loading or pulsing. This finding suggests that the recognition of T2 cells resulted from simple binding of the HER2/neu peptides to surface HLA molecules, from which it rapidly dissociated. T2 cells loaded with the constructs composed of signal sequence at the amino-terminus of the peptide HER2/neu₇₈₉₋₇₉₇ were recognized by CTL up to three days after loading (FIG. 7, left column).

TABLE 36
HER2/neu-derived HLA-A2 restricted peptides

| | | | |
|---|---|---|---|
| PEPTIDES | SEQUENCE | LOCATION | REFERENCE |

| | | | |
|---|---|---|---|
| HER2/neu₄₈₋₅₆ | HLYQGCQVV | EXTRACELLULAR | Disb, M. (Cancer Res. 54:1071-6,1994) |
| | (SEQ ID NO:132) | | |
| HER2/neu₃₆₉₋₃₇₇ | KIFGSLAFL | EXTRACELLULAR | Fisk, B. (J.Exp.Med. 181:2109-17, 1995) |
| | (SEQ ID NO:133) | | |
| HER2/neu₆₅₄₋₆₆₂ | IISAVVGIL | TRANSMEMBRANE | Peoples, G. (P.N.A.S 92:432-6, 1995) |
| | (SEQ ID NO:134) | | |
| HER2/neu₇₈₉₋₇₉₇ | CLTSTVQLV | INTRACELLULAR | Disis, M. (CancerRes. 54:1071-6,1994) |
| | (SEQ ID NO:135) | | |

TABLE 37
Synthetic peptide constructs with HER2/neu₄₈₋₅₆

| | |
|---|---|
| DESIGNATION | PEPTIDE SEQUENCE |

| | |
|---|---|
| HER-HER2/neu₄₈₋₅₆ | HLYQGCQVV |
| | (SEQ ID NO:132) |
| ES-HER₄₈₋₅₆ | MRYMILGLLALAAVCSA HLYQGCQVV |
| | (SEQ ID NO:136) |
| HER₄₈₋₅₆-ES | HLYQGCQVV MRYMILGLLALAAVCSA |
| | (SEQ ID NO:137) |
| IS-HER₄₈₋₅₆ | MTNKCLLQIALLLCFSTTALS HLYQGCQVV |
| | (SEQ ID NO:138) |
| HER₄₈₋₅₆-IS | HLYQGCQVV MTNKCLLQIALLLCFSTTALS |
| | (SEQ ID NO:139) |
| HER₄₈₋₅₆-IN-ES | MR HLYQGCQVV AAVCSA |
| | (SEQ ID NO:140) |
| HER₄₈₋₅₆-IN-AF | MA HLYQGCQVV AAAAAG |
| | (SEQ ID NO:141) |

Synthetic peptide constructs:
1. Peptide antigen HER2/neu₄₈₋₅₆
2. Adenoviral signal sequence ES attached to the atnino-terminus of HER2/neu₄₈₋₅₆
3. Adenoviral signal sequence ES attached to the carboxy-terminus of HER2/neu₄₈₋₅₆
4. Interferon signal sequence IS attached to the amino-terminus of HER2/neu₄₈₋₅₆
5. Interferon signal sequence IS attached to the carboxy-terminus of HER2/neu₄₈₋₅₆
6. Peptide antigen HER2/neu₄₈₋₅₆ replacing the hydrophobic portion of ES
7. Peptide antigen HER2/neu₄₈₋₅₆ incorporated into an artificial signal sequence - AF

TABLE 38

Synthetic peptide constructs with
HER2/neu₃₆₉₋₃₇₇

| | |
|---|---|
| DESIGNATION | PEPTIDE SEQUENCE |

| | |
|---|---|
| HER-HER2/neu₃₆₉₋₃₇₇ | KLFGSLAFL |
| | (SEQ ID NO:133) |
| ES-HER2₃₆₉₋₃₇₇ | MRYMILGLLALAAVCSA KIFGSLAFL |
| | (SEQ ID NO:142) |
| HER2₃₆₉₋₃₇₇-ES | KIFGSLAFL MRYMILGLLALAAVCSA |
| | (SEQ ID NO:143) |
| IS-HER2₃₆₉₋₃₇₇ | MTNKCLLQIALLLCFSTTALS KIFGSLAFL |
| | (SEQ ID NO:144) |
| HER2₃₆₉₋₃₇₇-IS | KIFGSLAFL MTNKCLLQIALLLCFSTTALS |
| | (SEQ ID NO:145) |
| HER2₃₆₉₋₃₇₇-IN-ES | M R KIFGSLAFL A A V C S A |
| | (SEQ ID NO:146) |
| HER2₃₆₉₋₃₇₇-IN-AF | M A KIFGSLAFL A A A A A G |
| | (SEQ ID NO:147) |

TABLE 39

Synthetic peptide constructs with
HER2/neu₆₅₄₋₆₂₂

| | |
|---|---|
| DESIGNATION | PEPTIDE SEQUENCE |

| | |
|---|---|
| HER-HER2/neu₆₅₄₋₆₂₂ | IISAVVGIL |
| | (SEQ ID NO:134) |
| ES-HER₆₅₄₋₆₂₂ | MRYMILGLLALAAVCSA IISAVVGIL |
| | (SEQ ID NO:148) |
| HER₆₅₄₋₆₂₂-ES | IISAVVGIL MRYMILGLLALAAVCSA |
| | (SEQ ID NO:149) |
| IS-HER₆₅₄₋₆₂₂ | MTNKCLLQIALLLCFSTTALS IISAVVGIL |
| | (SEQ ID NO:150) |
| HER₆₅₄₋₆₂₂-IS | IISAVVGIL MTNKCLLQIALLLCFSTTALS |
| | (SEQ ID NO:151) |
| HER₆₅₄₋₆₂₂-IN-ES | M R IISAVVGIL A A V C S A |
| | (SEQ ID NO:152) |
| HER₆₅₄₋₆₂₂-IN-AF | M A IISAVVGIL A A A A A G |
| | (SEQ ID NO:153) |

TABLE 40

Synthetic peptide constructs with
HER2/neu₇₈₉₋₇₉₇

| | |
|---|---|
| DESIGNATION | PEPTIDE SEQUENCE |

| | |
|---|---|
| HER-HER2/neu₇₈₉₋₇₉₇ | CLTSTVQLV |
| | (SEQ ID NO:135) |
| ES-HER₇₈₉₋₇₉₇ | MRYMILGLLALAAVCSA CLTSTVQLV |
| | (SEQ ID NO:154) |
| HER₇₈₉₋₇₉₇-ES | CLTSTVQLV MRYMILGLLALAAVCSA |
| | (SEQ ID NO:155) |
| IS-HER₇₈₉₋₇₉₇ | MTNKCLLQIALLLCFSTTALS CLTSTVQLV |
| | (SEQ ID NO:156) |
| HER₇₈₉₋₇₉₇-IS | CLTSTVQLV MTNKCLLQIALLLCFSTTA |
| | (SEQ ID NO:157) |
| HER₇₈₉₋₇₉₇-IN-ES | M R CLTSTVQLV A A V C S A |
| | (SEQ ID NO:158) |
| HER₇₈₉₋₇₉₇-IN-AF | M A CLTSTVQLV A A A A A G |
| | (SEQ ID NO:159) |

TABLE 41
⁵¹Cr-release assay using T2 cells pulsed with
HER2/neu-derived peptides as targets for CTL

| | E:T ratio | | | | | |
|---|---|---|---|---|---|---|
| | 50:1 | 25:1 | 12:1 | 6:1 | 3.1 | 1.5:1 |
| T2 | 1 | 2 | 1 | 0 | 1 | 0 |
| T2 pulsed with | 88 | 53 | 41 | 33 | 19 | 8 |
| HER2/neu48-56 | | | | | | |
| T2 pulsed with | 94 | 66 | 57 | 42 | 23 | 12 |
| HER2/neu₃₆₉₋₃₇₇ | | | | | | |
| T2 pulsed with | 91 | 71 | 59 | 38 | 26 | 13 |
| HER2/neu₆₅₄₋₆₆₂ | | | | | | |
| T2 pulsed with | 83 | 62 | 52 | 31 | 22 | 9 |
| HER2/neu₇₈₉₋₇₉₇ | | | | | | |

### EXAMPLE 11

### Signal Sequences Containing HER2/Neu Peptides

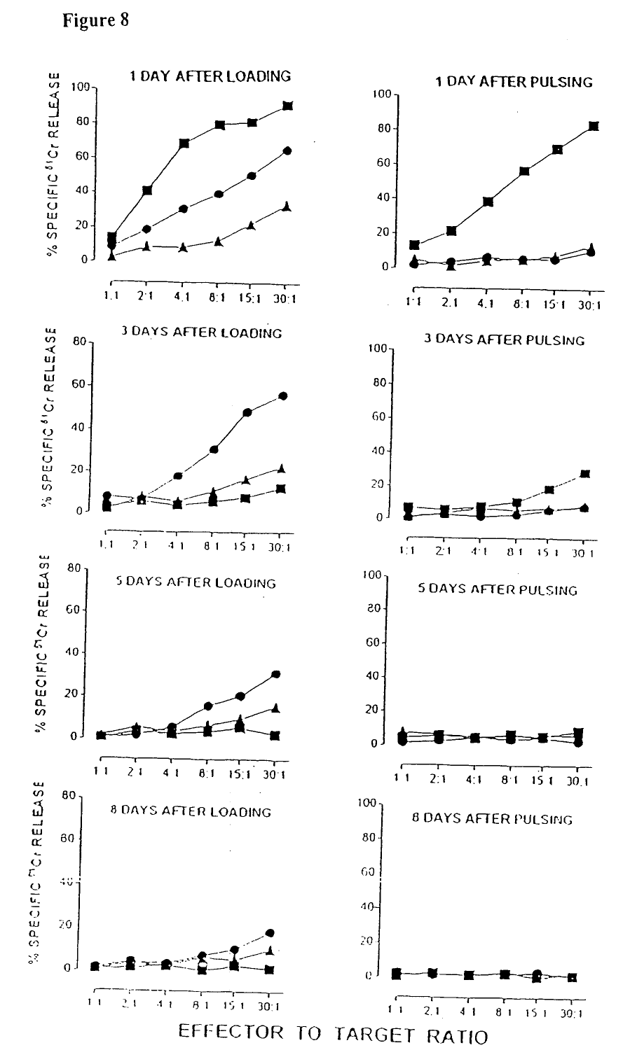

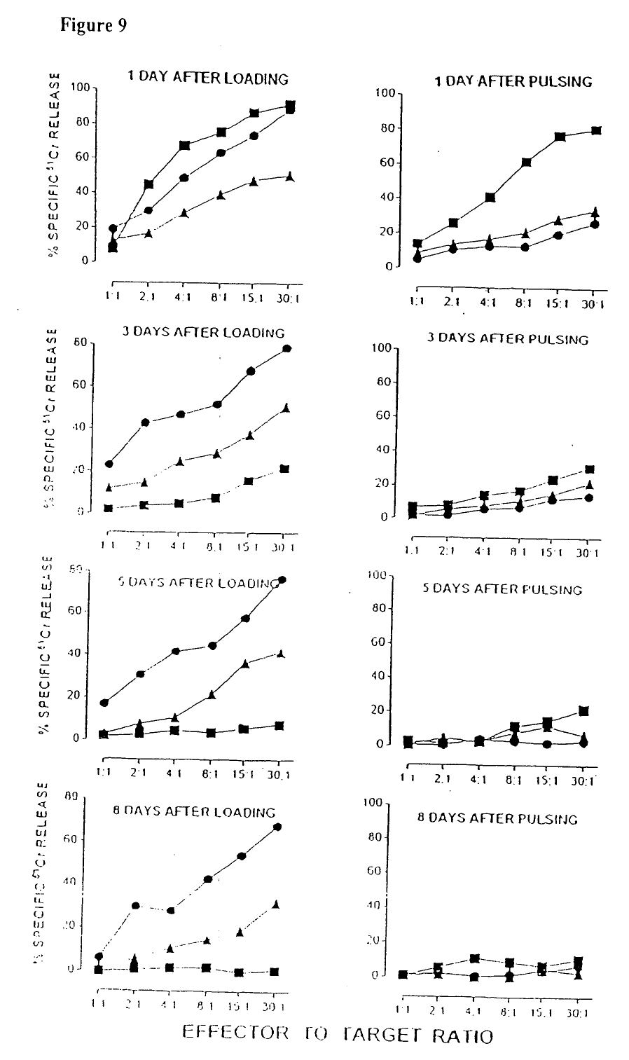

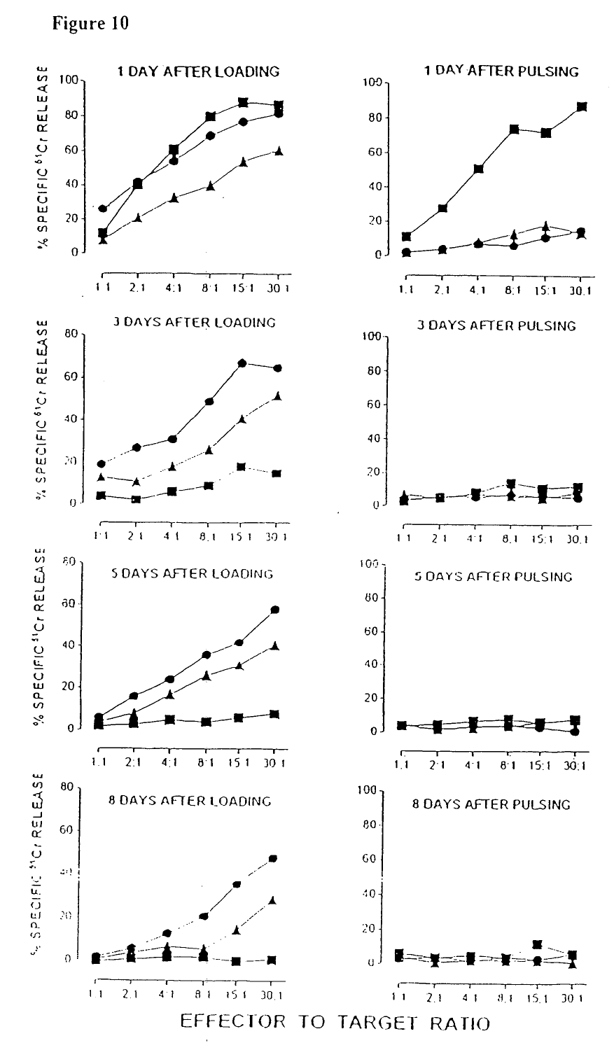

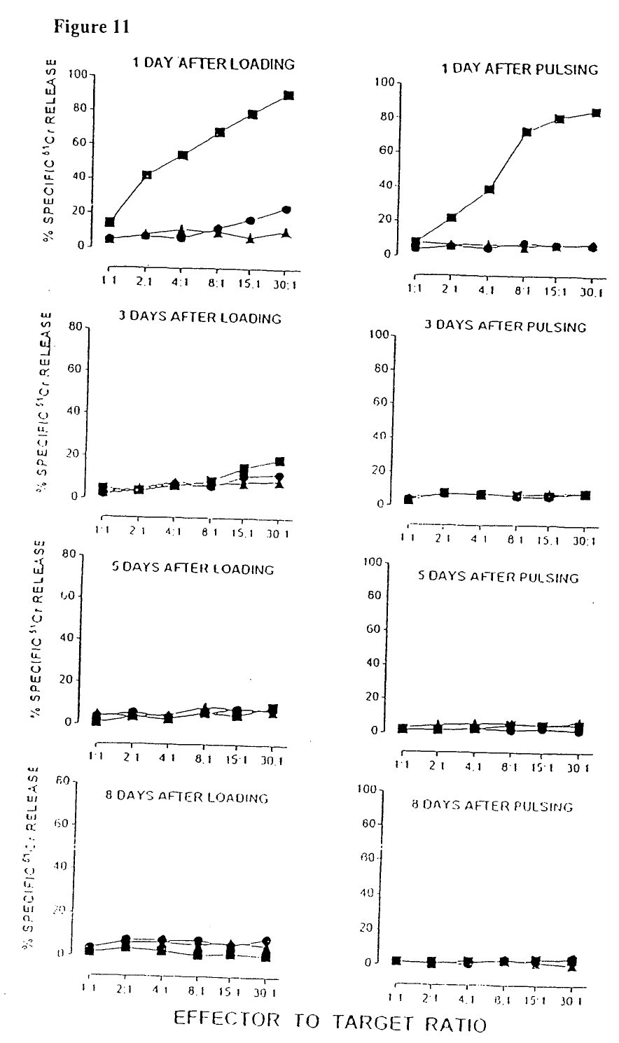

Since signal sequences do not contain specific amino acid residues other than a hydrophobic region of about eight residues, it was tested whether replacing this region with the hydrophobic HER2/neu peptides would result in a more efficient presentation of these epitopes (FIGS. 8-11). It was found that one of the two constructs of this type (HER-IN-AF) was the most efficient in facilitating the HER2/neu peptide presentation. Eight days after loading with the construct HER₃₆₉₋₃₇₇-IN-AF, T2 cells were still lysed with more than 60% specific ⁵¹Cr-release (FIG. 9). The constructs HER₄₈₋₅₆-IN-AF and HER₆₅₄₋₆₆₂-IN-AF were also effective (FIGS. 8 and 10). The second construct of this type (HER-IN-ES), although not as effective as HER-IN-AF, was able to facilitate the recognition of T2 cells (FIGS. 8-10). Pulsing of T2 cells with these constructs did not resulted in efficient presentation. Again, as in the first group of experiments, loading or pulsing with the minimal HER2/neu peptides resulted in recognition of T2 cells for only a short period of time.

In interferon gamma-release assays, 10⁵ HER2/neu-specific CTL were co-incubated with 10⁵ peptide-loaded T2 cells for 20 hours at 37° C. The concentrations of human interferon gamma in co-cultured supernatants were then determined by ELISA. The results of the ELISA experiments are shown in table 37 A-D. These findings are in parallel with the ⁵¹Cr-release experiments, and confirm that the most efficient constructs in facilitating the HER2/neu peptide presentation are the constructs of the type HER-IN-AF. As in the ⁵¹Cr-release experiments, the constructs with the peptides HER₃₆₉₋₃₇₇ and HER₆₅₄₋₆₆₂ were the most efficient, while the constructs with the peptide HER₇₈₉₋₇₉₇ were the least efficient, especially on days 5 and 8 after peptide loading.

TABLE 42
Release of IFNγ bv CTL after incubation with non-loaded or peptide-loaded T2 cells

| CTL elicited | | Stimulators in ELISA assays: T2 cells loaded with:^{a} | | | | | | |
|---|---|---|---|---|---|---|---|---|
| with: | — | HER | ES-HER | HER-ES | IS-HER | HER-IS | HER-IN-ES | HER-IN-AF |

A. Day 1 after peptide loading

| | | | | | | | | |
|---|---|---|---|---|---|---|---|---|
| HER2/neu₄₈₋₅₆ | 210^{b} | 2280 | 2894 | 418 | 3268 | 288 | 3368 | 3488 |
| HER2/neu₃₆₉₋₃₇₇ | 186 | 3120 | 3368 | 172 | 2120 | 212 | 2227 | 3288 |
| HER2/neu₆₅₄₋₆₆₂ | 121 | 2827 | 2667 | 144 | 2590 | 111 | 2929 | 3321 |
| HER2/neu₇₈₉₋ | 234 | 2924 | 1824 | 58 | 1717 | 69 | 246 | 296 |

B. Day 3 after peptide loading

| | | | | | | | | |
|---|---|---|---|---|---|---|---|---|
| HER2/neu₄₈₋₅₆ | 129^{b} | 488 | 1876 | 218 | 1264 | 148 | 1349 | 1229 |
| HER2/neu₃₆₉₋₃₇₇ | 143 | 127 | 2377 | 142 | 1818 | 112 | 2029 | 2401 |
| HER2/neu₆₅₄₋₆₆₂ | 111 | 429 | 2518 | 124 | 1990 | 99 | 2773 | 2981 |
| HER2/neu₇₈₉₋ | 215 | 317 | 526 | 78 | 315 | 78 | 312 | 327 |

C. Day 5 after peptide loading

| | | | | | | | | |
|---|---|---|---|---|---|---|---|---|
| HER2/neu₄₈₋₅₆ | 181^{b} | 134 | 953 | 99 | 943 | 155 | 988 | 1010 |
| HER2/neu₃₆₉₋₃₇₇ | 111 | 211 | 1073 | 121 | 1323 | 117 | 1663 | 1773 |
| HER2/neu₆₅₄₋₆₆₂ | 97 | 121 | 1245 | 137 | 1557 | 121 | 1699 | 1892 |
| HER2/neu₇₈₉₋ | 136 | 116 | 168 | 69 | 125 | 87 | 121 | 178 |

D. Day 8 after peptide loading

| | | | | | | | | |
|---|---|---|---|---|---|---|---|---|
| HER2/neu₄₈₋₅₆ | 116- | 177 | 589 | 101 | 614 | 121 | 228 | 718 |
| HER2/neu₃₆₉₋₃₇₇ | 93 | 89 | 592 | 118 | 545 | 103 | 690 | 878 |
| HER2/neu₆₅₄₋₆₆₂ | 115 | 167 | 581 | 83 | 615 | 76 | 671 | 881 |
| HER2/neu₇₈₉₋ | 88 | 91 | 110 | 98 | 104 | 59 | 107 | 117 |

^{a}CTL were coincubated with stimulator cells (non-loaded or peptide-loaded T2 cells) for 20 h. The concentration of IFNγ in coculture supernatants was then determined by ELISA.
^{b}IFNγ (pg/ml) - mean numbers of IFNγ release in triplicate wells with 10⁵ CTL/well.

### EXAMPLE 12

### Testing the Effectiveness of the Fusion Peptides with Breast Cancer Cells

It was tested whether the most effective signal sequence constructs, already selected in the experiments with the TAP-deficient T2 cells, can also improve HER2/neu antigen presentation in human breast cancer cells.

In this series of studies the HLA-A2+ human breast cancer cell line MCF-7 expressing high levels of HER2/neu and the cell line MDA-MB-231 expressing only basal levels of HER2/neu were used. HER/neu₃₆₉₋₃₇₇-specific CTL and HER2/neu₆₅₄₋₆₆₂-specific CTL failed to recognize the breast cancer cell line MDA-MB-231, although the same effectors specifically recognized T2 cells pulsed with HER2/neu₃₆₉₋₃₇₇, HER2/neu₆₅₄₋₆₆₂ and the cell line MCF7 expressing HER2/neu (Table 38). Thus, it was concluded that MDA-MB-231 cells do not express HER2/neu₃₆₉₋₃₇₇ and HER2/neu₆₅₄₋₆₆₂, and that this cell line was appropriate for the peptide-loading experiments described herein.

⁵¹Cr-release assays were used to test to see if the low HER2/neu-expressing breast cancer cells MDA-MB-231 can be recognized more efficiently by the HER2/neu-specific CTL after loading with the fusion peptides. Determination was also made by ELISA to see if the peptide-loaded breast cancer cells can induce release of interferon gamma by the HER2/neu-specific CTL.

The lysis of the tumor cells by the HER2/neu-specific CTL was monitored in a conventional ⁵¹Cr-labeling release assay. Briefly, peptide-loaded tumor cells were added to serially diluted effectors in 96-well microplates. After a 6-h incubation at 37° C., supernatants were harvested and counted in a gamma counter. Results are expressed as the percentage of specific lysis and determined as follows: [(experimental cpm−spontaneous cpm)/(maximum cpm−spontaneous cpm)]×100.

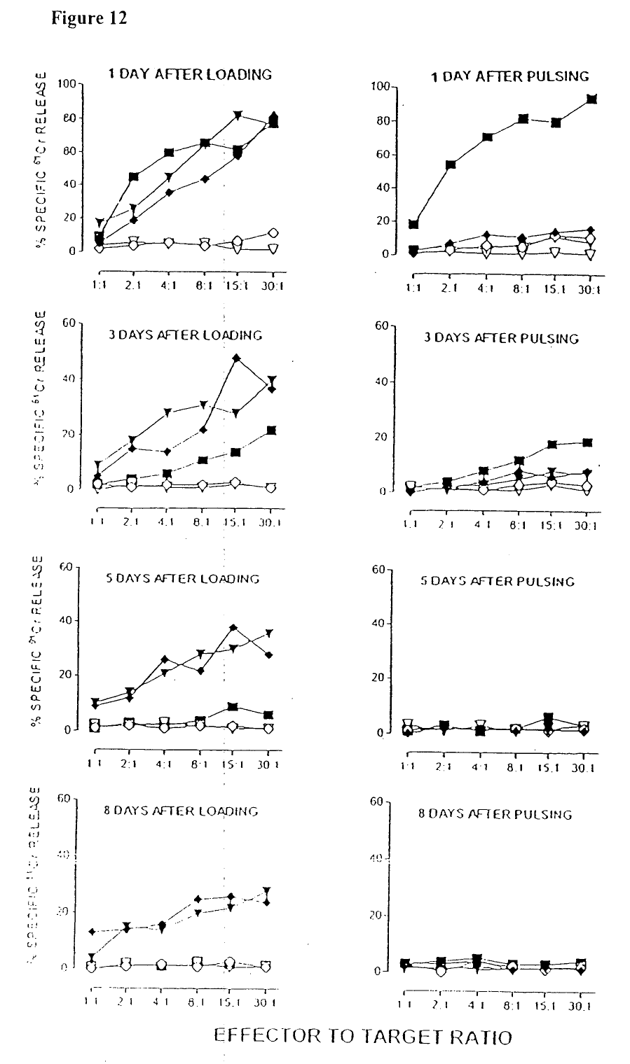

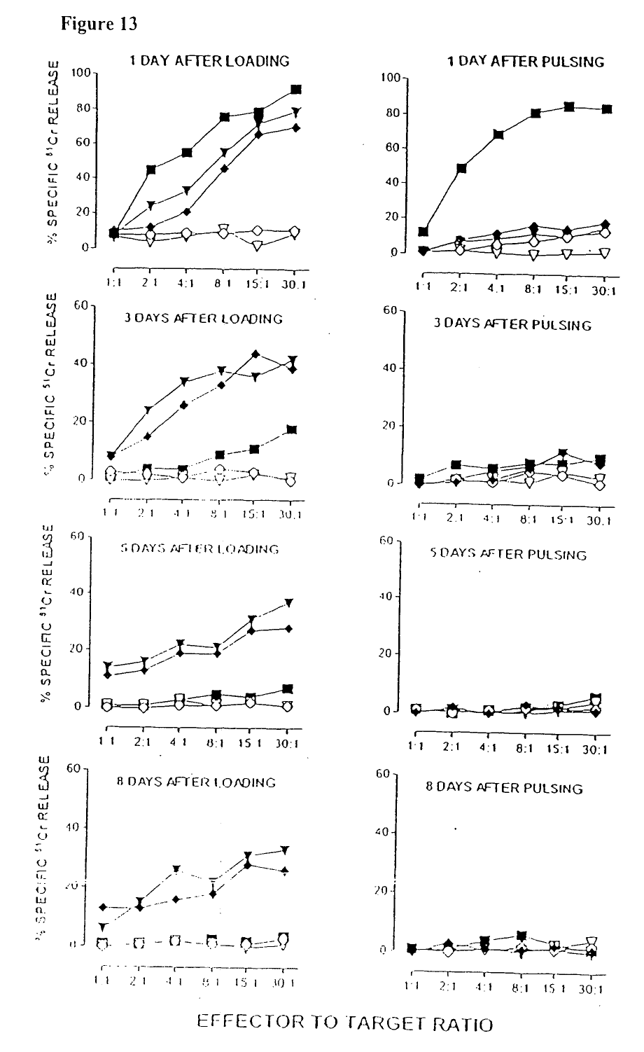

Peptide-loaded breast cancer cells MDA-MB-231 were tested for their ability to present HER2/neu peptides at different periods of time after loading or pulsing. Tumor cells loaded with the constructs composed of signal sequence at the amino-terminus of the peptides were recognized by CTL up to eight days after loading (FIGS. 12-13, left column). In contrast, constructs with carboxy-terminal position of the signal sequence were not efficient, even when ⁵¹Cr-release assays were performed immediately after loading. This recognition was not due to surface binding of these constructs since pulsing of the tumor cells with any of the constructs was not efficient (FIGS. 12-13, right column). Loading or pulsing with the minimal HER2/neu peptides resulted in a significant recognition and lysis of the tumor cells for only one day after loading or pulsing, followed by a rapid decrease of recognition on day 3 and complete lack of recognition on days 5 and 8 after loading or pulsing. This finding suggests that the recognition of the tumor cells resulted from simple binding of the HER2/neu peptides to surface HLA molecules, from which it rapidly dissociated.

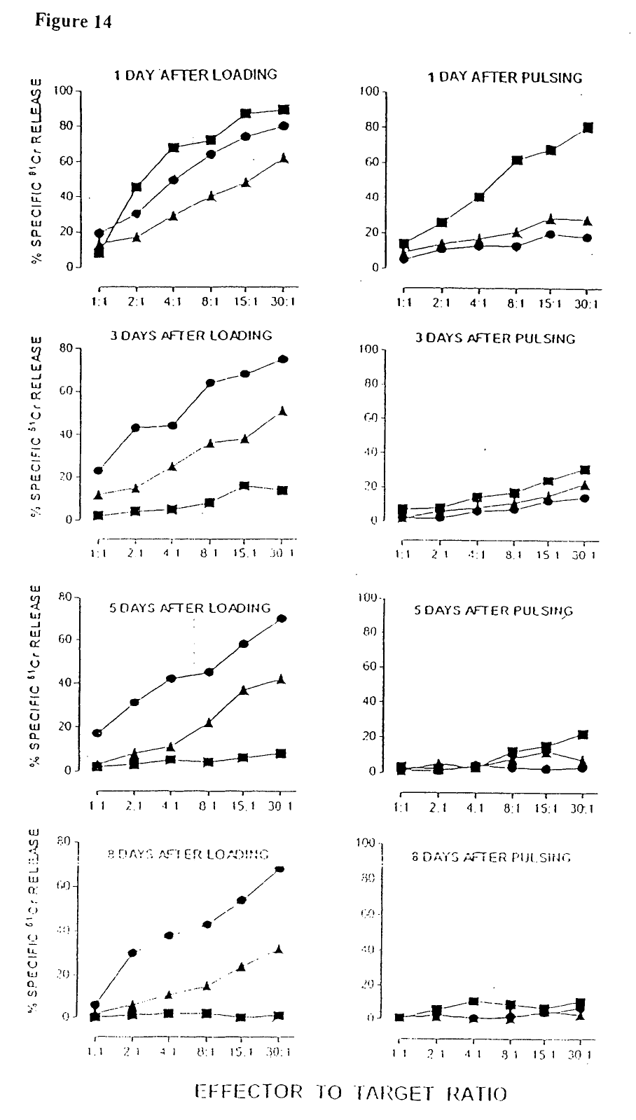

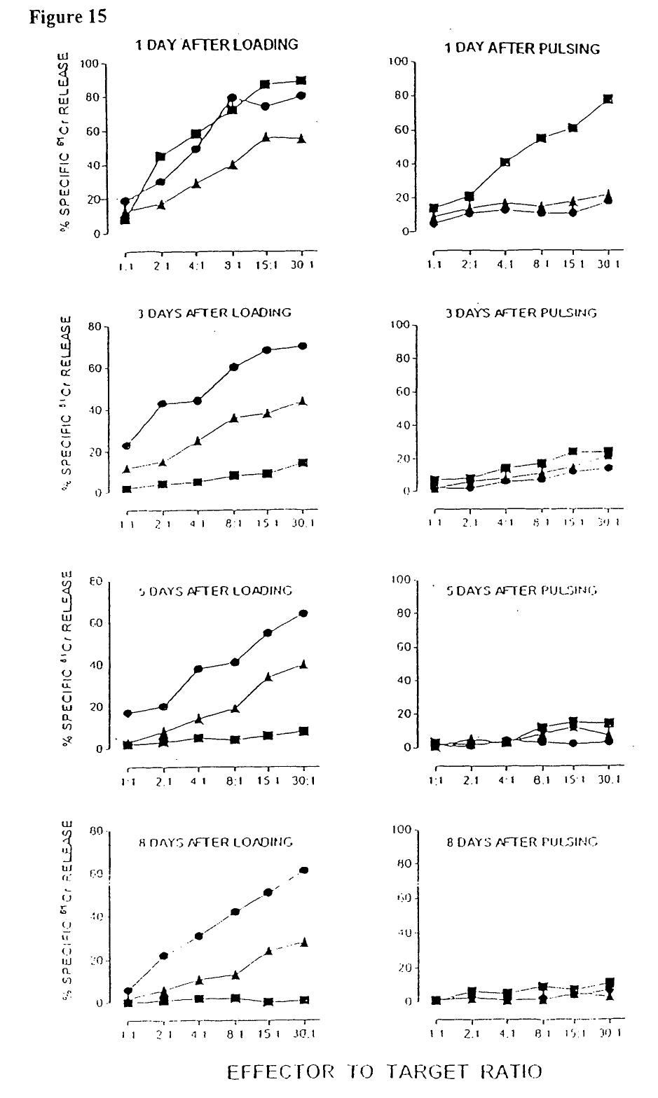

An experiment was also performed to test whether replacing the hydrophobic region of the signal sequences with the HER2/neu peptides would result in a more efficient presentation of these epitopes (FIGS. 14-15). The construct HER-IN-AF was found to be the most efficient in facilitating the HER2/neu peptide presentation. Eight days after loading with the construct HER₃₆₉₋₃₇₇-IN-AF, the tumor cells were still lysed (FIG. 14). The construct HER₆₅₄₋₆₆₂-IN-AF was also effective (FIG. 15). The second construct of this type (HER-IN-ES), although not as effective as HER-IN-AF, was able to facilitate the recognition of the tumor cells (FIGS. 14-15). Pulsing of the tumor cells with these constructs did not resulted in efficient presentation. Loading or pulsing with the minimal HER2/neu peptides resulted in recognition of the tumor cells for only a short period of time.

In interferon gamma release assays, 10⁵ HER2/neu-specific CTL were co-incubated with 10⁵ peptide-loaded tumor cells for 20 hours at 37° C. The concentration of human interferon gamma in co-cultured supernatants was then determined by ELISA. The results of the ELISA experiments are shown in Table 39 A-D. These findings are in parallel with the ⁵¹Cr-release experiments, and confirm that the most efficient constructs in facilitating the HER2/neu peptide presentation are the constructs of the type HER-IN-AF.

TABLE 43
Lack of recognition of breast cancer cell line MDA-MB-231 by
CTL reactive against HER2/neu₃₆₉₋₃₇₇ and HER2/neu₆₅₄₋₆₂₂

| | | | Percent ⁵¹Cr Released From^{a}: | | |
|---|---|---|---|---|---|
| Effectors | E:T | T2 | T2-pulsed^{b} | MCF7 | MDA-MB-231 |
| CTL₃₆₉₋₃₇₇ | 40:1 | 2 | 94 | 68 | 2 |
| | 20:1 | 1 | 73 | 33 | 1 |
| CTL_{654.662} | 40:1 | 1 | 71 | 58 | 2 |
| | 20:1 | 2 | 57 | 31 | 0 |

^{a}Cytotoxicity was evaluated in a 6-hour ⁵¹Cr-release assay.
^{b}T2 cells were pulsed with HER2/neu₃₆₉₋₃₇₇, or HER2/neu₆₅₄₋₆₂₂ at 1 μg/ml for 2 hours at 37° C., labeled with ⁵¹Cr and used as targets.

TABLE 44
Release of IFNγ by CTL after incubation with non-loaded
or peptide-loaded breast cancer cells MDA-MB-231

| CTL elicited | | Stimulators in ELISA assays: T2 cells loaded with:^{a} | | | | | | |
|---|---|---|---|---|---|---|---|---|
| with: | — | HER | ES-HER | HER-ES | IS-HER | HER-IS | HER-IN-ES | HER-IN-AF |

A. Day 1 after peptide loading

| | | | | | | | | |
|---|---|---|---|---|---|---|---|---|
| HER2/neu₃₆₉₋₃₇₇ | 177^{b} | 2820 | 3688 | 182 | 2126 | 224 | 2627 | 3381 |
| HER2/neu₆₅₄₋₆₂₂ | 153 | 2826 | 2767 | 188 | 2678 | 144 | 2727 | 3321 |

B. Day 3 after peptide loading

| | | | | | | | | |
|---|---|---|---|---|---|---|---|---|
| HER2/neu₃₆₉₋₃₇₇ | 138^{b} | 141 | 2417 | 187 | 1777 | 131 | 2187 | 2347 |
| HER2/neu₆₅₄₋₆₂₂ | 121 | 438 | 2622 | 138 | 1974 | 102 | 2666 | 2994 |

C. Day 5 after peptide loading

| | | | | | | | | |
|---|---|---|---|---|---|---|---|---|
| HER2/neu₃₆₉₋₃₇₇ | 122^{b} | 274 | 1278 | 137 | 1444 | 128 | 1778 | 1897 |
| HER2/neu₆₅₄₋₆₂₂ | 102 | 131 | 1445 | 135 | 1604 | 132 | 1708 | 1933 |

D. Day 8 after peptide loading

| | | | | | | | | |
|---|---|---|---|---|---|---|---|---|
| HER2/neu₃₆₉₋₃₇₇ | 102^{b} | 99 | 577 | 122 | 587 | 113 | 687 | 889 |
| HER2/neu₆₅₄₋₆₂₂ | 125 | 147 | 578 | 93 | 628 | 86 | 667 | 899 |

^{a}CTL were coincubated with stimulator cells (non-loaded or peptide-loaded MDA-MB-231 cells) for 20 h. The concentration of IFNg in coculture supernatants was then determined by ELISA.
^{b}IFNg (pg/ml) - mean numbers of IFNg release in triplicate wells with 10⁵ CTL/well.

### EXAMPLE 13

### Identification of the Mechanisms Involved in the Enhancement of Antigen Presentation by the Fusion Peptides

The goal of this set of experiments was to prove that the effective presentation of the loaded peptide constructs is a result of their efficient loading into the cytosol and not simple binding to the surface HLA molecules. The role of TAP in class I presentation in human cancer cells was also tested, along with a test of the efficiency of different signal peptides in cancer cells with different levels of TAP expression.

### EXAMPLE 14

### Probing the Mechanisms of Peptide Loading

To distinguish between loading of the peptides into the cytosol and simple binding of these peptides to the surface MHC molecules several approaches were used. First, β₂-microglobulin was removed from the surface of peptide-loaded tumor cells by acid stripping. It was found that acid-stripping solution with pH=3.5 was most efficient in decreasing the specific recognition of peptide-loaded cells. Second, pronase was used for complete enzymatic digestion of HLA molecules on the cell surface after loading in order to be able to detect the appearance of new internally formed HLA-peptide complexes on the cell surface, but not pulsing of the cells. Third, Brefeldin A (BFA), a metabolite of the fungus Eupenicillium brefeldianum, was used which specifically blocks protein transport from the ER to Golgi apparatus.

It was found that Brefeldin A specifically blocks the recognition of the peptide-loaded tumor cells by the HER2/neu-specific CTL. In contrast, the acid stripping and the treatment with pronase was not able to block antigen recognition for more than 24 hours (Table 40). These experiments confirmed that the antigenic peptides were introduced into the cytosol of the cells, resulting in a prolonged and more efficient antigen presentation.

TABLE 45
Mechanisms of peptide loading: Recognition of breast
cancer cells MDA-MB-231 by CTL reactive against
HER2/neu_{369•377} and HER2/neu₆₅₄₋₆₂₂

| | | Percent ⁵¹Cr Released From | | | |
|---|---|---|---|---|---|
| | | MDA-MB-231 cells treated with^{a}: | | | |
| | | | | | non- |
| Effectors | E:T | acid | pronase | brefeldin | treated |
| CTL₃₆₉₋₃₇₇ | 40:1 | 62 | 59 | 18 | 62 |
| | 20:1 | 41 | 33 | 7 | 39 |
| CTL₆₅₄₋₆₆₂ | 40:1 | 61 | 61 | 13 | 58 |
| | 20:1 | 28 | 37 | 6 | 30 |

^{a}Cytotoxicity was evaluated in a 6-hour ⁵¹Cr-release assay 3 days after peptide loading

### EXAMPLE 15

### Inducing a Functional Blockade of TAP by ICP47

Another aspect in these studies was to determine the mechanisms of enhancement of the antigen presentation by the fusion peptides in human tumor cell lines. Therefore, a new test system was developed utilizing the Herpes Simplex virus (HSV) protein ICP47. ICP47 is a cytoplasmic protein, which interferes with antigen presentation by physically associating with TAP within the cell and inhibiting peptide transport across the ER-membrane. By transfecting the ICP47 gene into several cancer cell lines a novel system for screening different fusion peptides for TAP-independent translocation of peptide antigens through the ER-membrane was generated.

The breast cancer cell line MCF7 was transfected with ICP47, and observed permanent block of the function of TAP, and therefore lack of recognition of these cells by the CTL, which normally recognize and kill them. To select the sequences most effective in translocation of antigenic peptides across the ER-membrane of the breast cancer cells, the ICP47-transfected cells were loaded with several fusion peptides with different signal sequences. The expression of these antigens was detected by ⁵¹Cr-release assays. It was found that only the most efficient peptide constructs—HER₃₆₉₋₃₇₇-IN-AF and HER₆₅₄₋₆₆₂-IN-AF—were able to restore the antigen presentation in the ICP47-transfected breast cancer cells (Table 41). This confirms that the signal sequence approach is very effective in improving antigen presentation, even in tumor cells with deficiency of antigen processing/presentation.

TABLE 46
Mechanisms of peptide loading: Recognition of
ICP47-transfected cells MCF7 by CTL reactive against
HER2/neu_{369•377} and HER2/neu₆₅₄₋₆₂₂

| | | | Percent ⁵¹Cr Released From | | | | |
|---|---|---|---|---|---|---|---|
| | | | MCF7 cells loaded with^{a}: | | | | |
| | | | MCF7- | ES- | IS- | HER- | HER- |
| Effectors | E:T | MCF7 | ICP47 | HER | HER | IN-ES | IN-AF |
| CTL₃₆₉₋₃₇₇ | 40:1 | 62 | 3 | 1 | 2 | 4 | 58 |
| | 20:1 | 29 | 3 | 2 | 3 | 3 | 24 |
| CTL₆₅₄₋₆₆₂ | 40:1 | 74 | 4 | 3 | 5 | 2 | 66 |
| | 20:1 | 31 | 2 | 2 | 3 | 2 | 22 |

^{a}Cytotoxicity was evaluated in a 6-hour ⁵¹Cr-release assay 3 days after peptide loading

### EXAMPLE 16

### Loading of Dendritic Cells with the Fusion Peptides

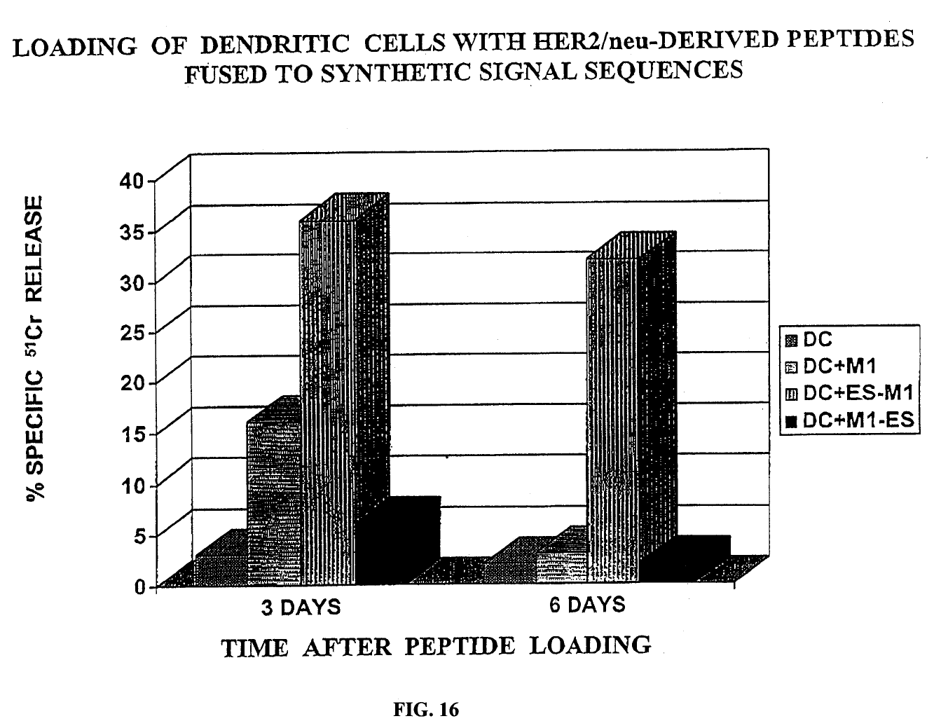

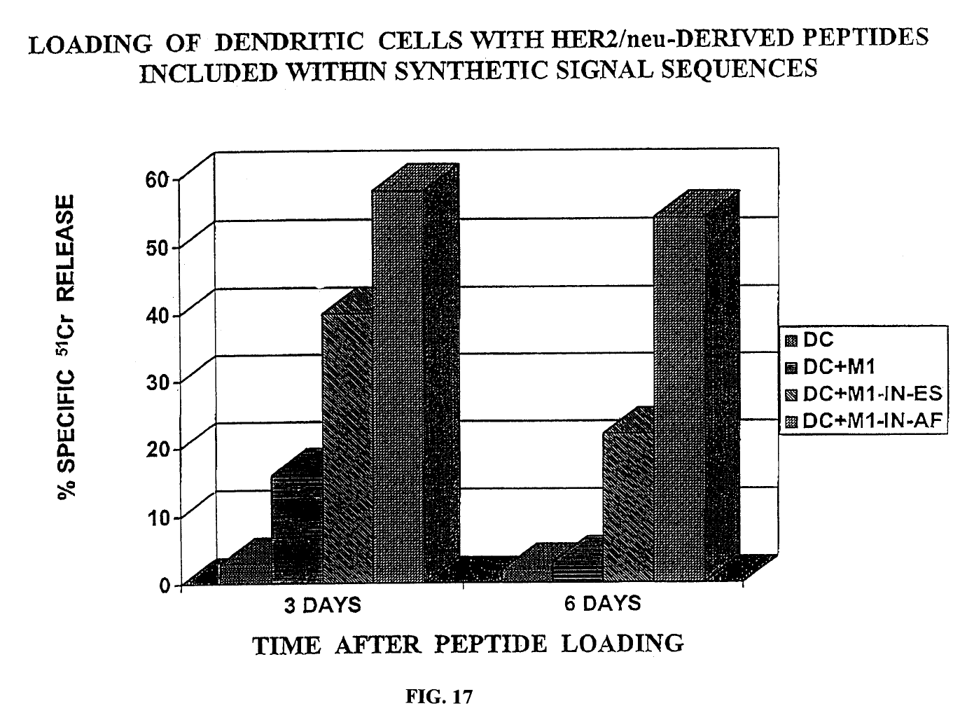

Human dendritic cells (DC) derived from healthy donors were utilized. The nonamer HER2/neu peptides were introduced alone, fused to, or included within, synthetic signal sequences into the cytosol of DC with a technology called “osmotic lysis of pinocytic vesicles.” With a standard ⁵¹Cr-release assay, the ability of HER2/neu-specific tumor infiltrating lymphocytes (TIL) to recognize peptide-loaded DC at various intervals after loading was tested. Significant lysis of DC loaded with a peptide construct composed of a signal sequence fused to the amino-terminus was observed, but not the carboxy-terminus of HER2/neu peptide (FIG. 16). Of all constructs tested, DC loaded with the HER2/neu peptide included within an artificial signal sequence were recognized most efficiently, for at least 6 days after loading (FIG. 17). DC loaded with the minimal peptide were only marginally recognized. In all of the experiments described herein, non-loaded DC were not recognized by the HER2/neu specific CTL. These studies suggest that with signal sequences combined with minimal antigenic peptides, it may be possible to enhance antigen-presentation and stimulation of cytotoxic T lymphocytes. This approach may facilitate the development of synthetic peptide vaccines for human cancer.

### EXAMPLE 17

### Nanoparticle-Based Synthetic Vaccines for Cancer and Infectious Diseases

A major obstacle affecting the activity of peptides that function intracellularly is the cytoplasmic delivery. Biomolecules usually enter cells via fluid-phase or receptor-mediated endocytosis, and are initially localized in the endosomal compartment. A high percentage of these biomolecules are subsequently sent to lysosomes, resulting in high levels of protein degradation and thus limiting antigen delivery. Accordingly the design and synthesis of specialized carriers that can enhance the intracellular delivery of biotherapeutics, in particular to overcome the important barrier of lysosomal trafficking, is important for vaccine development.

A new strategy will be implemented for the design and synthesis of polymeric nanoparticles that enhance the cytoplasmic delivery of the peptide vaccines into the antigen-presenting cells by disrupting the endosomal membrane at the acidic pH of the endosome. These acid-sensitive nanoparticles will be designed to disrupt endosomes and deliver protein antigens into the cytoplasm of antigen-presenting cells (APC) for class I antigen presentation. The nanoparticles will be chemically stable at pH 7.4, but will degrade into linear polymer chains and small molecules under mildly acidic conditions.

It is hypothesized that tumor/pathogen antigen-derived peptide vaccines encapsulated in acid-sensitive nanoparticles will induce potent and specific CTL responses against cancer and infectious diseases. This approach may provide a potential avenue for vaccine development using the cancer-associated antigens and pathogen-associated antigens described herein.

The development of nanoparticle-based vaccines is innovative and holds great promise. Like the biological systems, these nanoparticles combine targeting elements that direct cellular uptake, together with the sensing of pH changes within the endosome to activate membrane destabilization and cytosolic delivery. The intrinsic modular design of these nanoparticle-based vaccines makes it possible to customize the targeting and membrane destabilizing activities for a wide range of biotherapeutics and vaccine applications. These vaccines might be used directly to immunize patients with cancer or infectious diseases. In addition, they may be used to generate and expand in vitro CTL for adoptive transfer therapies.

### EXAMPLE 18

### Survivin-Based Synthetic Vaccines for Immunotherapy of Brain Tumors

Gliomas are among the most common tumors of the central nervous system (CNS). Even with conventional treatments, including surgery, radiation, and chemotherapy, the median survival time for patients with gliomas, is only one year. As these tumors are incurable, the aim of the current conventional treatments is to improve the neurological deficits and to increase survival while maintaining the best possible quality of life. It is has recently been discovered that with Gliomas, there is a significant trafficking of activated T cells through the CNS, and that T cells primed by tumor cells in the periphery can recirculate and reach the brain to mediate their anti-tumor effects.

A newly described inhibitor of apoptosis, survivin, has been found to induce in vitro survivin-specific effector T lymphocytes in healthy donors, as well as in patients with cancer. Most importantly, spontaneous T cell reactivity against survivin in patients with leukemia, melanoma and breast cancer has been observed. The over-expression of survivin in most gliomas and many other human tumors suggests a general role of apoptosis inhibition during tumor progression. Survivin may be an ideal target for the immunotherapy of gliomas because of its strong expression in most gliomas, little or no expression in adult tissues, and its essential role for the survival of the tumor cells.

The development strategy will be to (i) identify and obtain class I-restricted immunogenic survivin-derived peptides, (ii) generate in vitro survivin-specific CTL lines and clones from healthy volunteers and from patients with glioma, (iii) test the ability of the survivin-specific CTL to kill glioma tumor cells in vitro in a class I-restricted and survivin-dependent fashion, and (iv) enhance the stability and immunogenicity of the survivin-derived synthetic vaccines. Several survivin peptides have already been observed to expand precursor CTL in PBMC of healthy individuals and induce MHC class I-restricted, peptide-specific CTL responses. Therefore, it is hypothesized that survivin-derived peptides may be used for vaccination of HLA-A2.1 positive cancer patients.

The identification of immunogenic peptides derived from survivin, a widely expressed tumor antigen, is innovative and holds great promise. Identification of immunogenic survivin peptides will allow for the development of synthetic vaccines for patients with glioma. Furthermore, immunogenic survivin peptides will be used to generate and expand in vitro CTL for adoptive transfer therapies, or for dendritic cell-based immunotherapy.

### EXAMPLE 19

### Polymeric Nanoparticles for Vaccine Delivery

Cell lines—T2 cell [Salter, 1986 #21; Salter, 1986 #21] was purchased from ATCC (Manassas, Va.). Tumor-infiltrating lymphocytes (TILs) TIL1235 and TIL771 were kindly provided by Dr. John R Wunderlich (NIH/NCI, Bethesda, Md.). T2 cell line was maintained in RPMI 1640 supplemented with 10% FCS, 2 mM glutamine, 50 U/ml penicillin and 50 mg/ml streptomycin. TIL cell lines were maintained in RPMI medium supplemented with 10% human AB+ serum, antibiotics, and 6000 IU IL-2/ml.

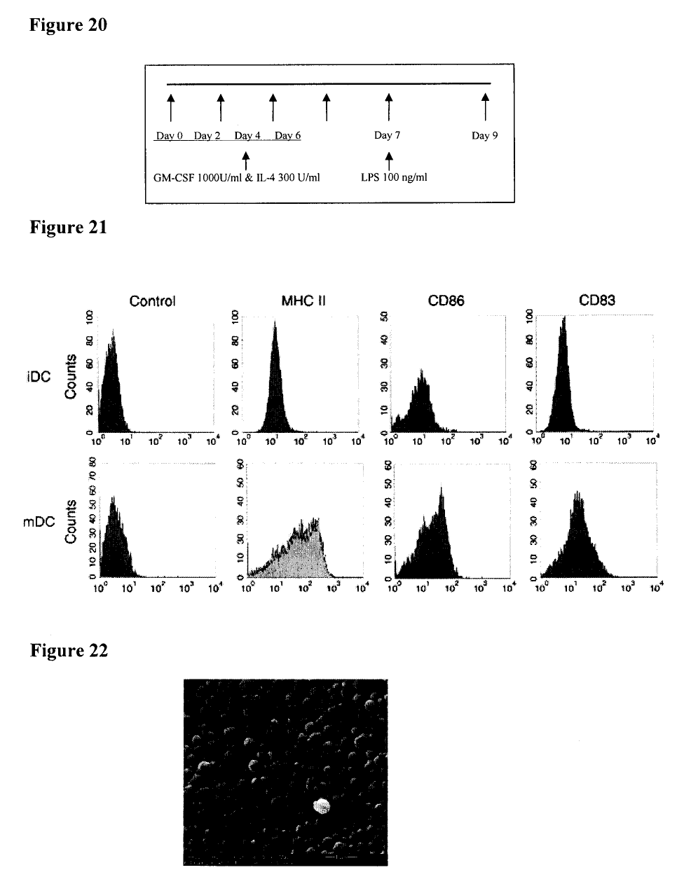

Generation of human Dendritic Cells—Peripheral blood mononuclear cells (PBMC) were isolated from healthy donors by Ficoll density-gradient centrifugation. HLA-A2-positive PBMC were included in these experiments. These PBMC were allowed to adhere in 6-well plates, or T75 tissue culture flasks, at a density of 6-8×10⁶ cells/ml for 1 h in a 37° C., 5% CO₂ humidified incubator, in RPMI 1640 medium with 1% heat inactivated human AB+ serum. The non-adherent lymphocytes were decanted and the adherent cells were washed gently with pre-warmed PBS for four times. The adherent cells were cultured in RPMI 1640 medium containing 10% human AB+ serum, 1000 U/ml GM-CSF and 300 U/ml IL-4. Subsequently, 1000 U/ml GM-CSF and 300 U/ml IL-4 were added on day 0, day 2, day 4 and day 6. Immature human DCs were collected and incubated with nanoparticles containing Mart-1 peptide and/or the fluorescence dye coumarin 6 for 1 h; 100 ng/ml of Lipopolysacchride (LPS) was added on day 7 without any cytokines (FIG. 20). Two days later, mature human DCs were harvested and tested by Fluorescence Activated Cell Sorting (FACS).

Synthetic peptides—The melanoma-associated peptide antigen Mart-1:27-35 with the sequence of AAGIGILTV (SEQ ID NO: 194) was used in this study. It was supplied by GenScript Corp. (Piscataway, N.J.). The identity of the Mart-1 peptide was determined by amino acid analysis.

Peptide and nanoparticle loading—Mart-1 peptide was dissolved in 100% DMSO at a concentration of 20 mg/ml. In this study the stock solution of peptide was diluted to a concentration of 0.5 μg/ml in RPMI 1640. Then, 2×10⁴ human DCs, in a volume of 80 μl, were loaded into a 96-well filtration plate (Millipore Corp, Bedford, Mass.). Twenty μl of Mart-1 peptide with a working concentration of 0.5 μg/ml was then added for peptide loading. The nanoparticle loading was performed by resuspending 2×10⁴ human DCs into the nanoparticle solution at concentration of 100 μg/ml. The DCs were then incubated with the nanoparticles for 1 hour at 37° C. and washed 3 times with warm RPMI medium before the in vitro experiments.

PLGA Polymer and Chemicals—PLGA (MW 23,000, copolymer ratio 50:50) was purchased from Birmingham Polymers, Inc. (Birmingham, Ala.). Albumin from rat serum (RSA), bovine serum albumin (BSA, Fraction V) and Poly(vinyl alcohol) (PVA, average MW 30,000-70,000) were purchased from Sigma-Aldrich (St. Louis, Mo.). Coumarin 6 was purchased from Polyscience, Inc. (Warrington, Pa.). All salts used in the preparation of buffers were from Fisher Scientific (Pittsburgh, Pa.). All aqueous solutions were prepared with distilled and deionized water (Water pro plus, Labconco, Kansas City, Mo.).

Antibodies and cytokines—Anti-human IFN-γ (mAb 1-D1K) was purchased from Mabtech Inc. (Mariemont, Ohio) for the ELISpot assay. FITC-anti-human HLA-A2, PE-anti-human HLA-DR, PE-anti-human CD83, FITC-anti-human CD80, FITC-anti-human CD86, FITC-mouse IgG1, PE mouse IgG1, and PE mouse IgG2a were all purchased from BD Pharmingen (San Diego, Calif.). GM-CSF (LEUKINE) was purchased from BERLEX Laboratories Inc. (Richmond, Calif.); Interleukin 4 (IL-4) was purchased from PeproTech Inc. (Rocky Hill, N.J.). IL-2 was purchased from Chiron Corp. (Emeryville, Calif.).

Nanoparticle formulation—Nanoparticles containing RSA, Mart-1 peptide and coumarin 6 were formulated using a double emulsion-solvent evaporation technique as described previously (Davda, 2002 #22). For optimizing the amount of Mart-1 peptide included in PLGA nanoparticles in each batch, 300 μg, 600 μg or 1 mg of Mart-1 peptide was loaded into the PLGA polymer, respectively when making Mart-1 nanoparticles. In brief, a solution of 30 mg PLGA polymer and 300 μg, 600 μg or 1 mg of Mart-1 peptide in 1 ml of chloroform was emulsified in 6 ml of 2% w/v aqueous solution of PVA to form an oil-in-water emulsion, respectively. An aqueous solution of RSA (60 mg/ml, 200 μl) was emulsified in a PLGA solution (30 mg in 1 ml chloroform) using a probe sonicator (55 W for 2 min) (Sonicator® XL, Misonix, N.Y.). The water-in-oil emulsion that formed was further emulsified into 6 ml of 2% w/v aqueous solution of PVA by sonication (55 W for 5 min). This formed a multiple water-in-oil-in-water emulsion. The multiple emulsions were stirred for 18 h at room temperature followed by 1 h in a desiccator under vacuum to remove the residual chloroform. Nanoparticles were recovered by ultracentrifugation (35,000 rpm for 30 min at 4° C., Optima™ LE-80K, Beckman, Palo Alto, Calif.), washed twice with distilled water to remove PVA, un-trapped RSA and coumarin 6, and then lyophilized (−80° C. and <10 μm mercury pressure, Sentry™, Virtis, Gardiner, N.Y.) for 48 h to obtain a dry powder. Dry lyophilized nanoparticle samples were stored in a dessicator at 4° C. and were reconstituted in a suitable medium (buffer or cell culture medium) prior to an experiment.

To determine cellular uptake of nanoparticles, the nanoparticle formulation contained a fluorescent dye (coumarin 6). The dye (100 μg of coumarin 6 in 100 μl chloroform) was added to the polymer solution prior to emulsification of one batch. The incorporated dye acts as a probe for nanoparticles and thus can be used to quantitatively determine the intracellular uptake of nanoparticles (Panyam, 2002 #23).

Particle size measurement—To determine the nanoparticle size and size distribution, the nanoparticle sample was subjected to particle size analysis using a scanning electronic microscope (FEI Company, Sunnyvale, Calif.).

Nanoparticles uptake study—Based on previously published data (Davda, 2002 #22), the nanoparticles containing Mart-1 peptide were used with a concentration of 100 μg/ml incubated for 1 h with human DCs. To study nanoparticle uptake, human imDCs were seeded on a 12-well plate with sterile cover slips at a DC number of 50,000 per well. The DCs were allowed to attach to the cover slips overnight in the presence of GM-CSF (1000 U/ml) and IL-4 (300 U/ml). The medium in the wells was replaced with freshly prepared nanoparticle suspension containing coumarin 6 and the plates were incubated for 1 h. The images were then taken under a fluorescent microscope.

On day 9 human mDCs, were phenotyped with the monoclonal markers HLA-DR, CD80, CD83, and CD86. Human DCs (5×10⁵) were incubated with antibodies at 4° C. for 30 min, then washed with PBS and supplemented with 1% BSA three times. The DC pellet was resuspended in 0.5 ml FACS buffer (PBS containing 0.5% BSA) and staining analysis was carried out by FACS (Becton Dickinson USA).

Enzyme-Linked Immunospot Assay (ELISpot)—The enumeration of cytokine secreting cells was carried out using commercial ELISpot-kits for IFN-γ (Mabtech, Mariemont, Ohio). On day 7 of culture immature human DCs were stimulated with lipopolysaccharide (LPS, Sigma-Aldrich), at 100 ng/ml for 48 h. Subsequently, these human DCs were washed and resuspended in RPMI 1640 medium. DCs (2×10⁴/well) were distributed in 10 wells of a nitrocellulose bottom ELISPOT plate (Millipore, Billerica, Mass.) that had previously been coated overnight with 10 μg/ml of monoclonal human anti-IFN-γ antibody (Mabtech, Stockholm, Sweden). Mart-1:27-35 peptide (AAGIGILTV (SEQ ID NO: 194), synthesized by GenScript Corp. Piscataway, N.J.) was added to the wells at a final concentration of 0.5 μg/ml. This peptide corresponds to HLA-A2-restricted CTL epitope. Next, responder cells (TIL1235) were added to the DC cultures at a ratio of 1:1 of DC to responder cells. Cells were cultured for 24 h at 37° C. ELISpot plates were developed using biotinylated anti human IFN-γ (2 μg/ml, avidin-bound biotinylated horseradish peroxidase (VectorLaboratories, Burlingame, Calif.) and AEC substrate for peroxidase (VectorLaboratories, Burlingame, Calif.). The plates were scanned and the spots were counted automatically using the image analysis system ELISpot reader (CTL Analyzers LLC, Cleveland Ohio). For optimization of the amount of Mart-1 in the nanoparticles, different nanoparticle batches containing various amount of Mart-1 were included in this experiment. The layout is as follows: (1) DCs+TIL1235; (2) DCs+Mart-1+TIL1235; (3) T2+Mart-1+TIL1235; (4) DCs+CNP+TIL1235 (CNP: control nanoparticle without any peptide); (5) DCs+NPs-Mart-1-300 μg+TIL1235; (6) DCs+NPs-Mart-1-600 μg+TIL 1235; (7) DCs+NPs-Mart-1-1 mg+TIL1235.

Statistical analysis—Results are expressed as the mean±SD. Statistical analysis was conducted by unpaired Student's t-tests, and was performed using Microsoft excel. A p value <0.05 was considered statistically significant.

Generation and characterization of human dendritic cells—PBMCs were isolated from buffy coats (San Diego blood bank, San Diego, Calif.) by using Ficoll-Hypaque density gradient centrifugation, and cultured in complete medium (RPMI 1640 containing 2 mM L-glutamine, 50 units/ml penicillin, 50 μg/ml streptomycin, sodium pyruvate 1 mM, and 10% heat-inactivated human AB serum). In his study, the human DCs were generated based on the following schedule (FIG. 20), and the DC markers were tested by FACS. Isolated PBMC with a cell density of 6-8×10⁶ cells/ml were cultured in RPMI 1640 containing 1% human AB serum, at 37° C. in the incubator for 1 h. The nonadherent cells were removed, and the adherent cells were cultured in RPMI 1640 complete medium containing 1000 U/ml GM-CSF and 300 U/ml IL-4. After rinsing with PBS four times on day 0, the same amount of GM-CSF and IL-4 was added on day 2, day 4 and day 6. LPS (100 ng/ml) was added to the culture medium on day 7 for maturation. Two days later (on day 9), the human mature DCs (mDCs) were harvested and tested by FACS (FIG. 21).

Characterization of PLGA nanoparticles containing peptide Mart-1:27-35—The PLGA nanoparticles made in this study, where analyzed using a scanning electron microscope (SEM). This demonstrated a size distribution of nanoparticles in size ranges: 181-282 nm. This fraction had a mean diameter of 215.46±48.6 nm (FIG. 22).

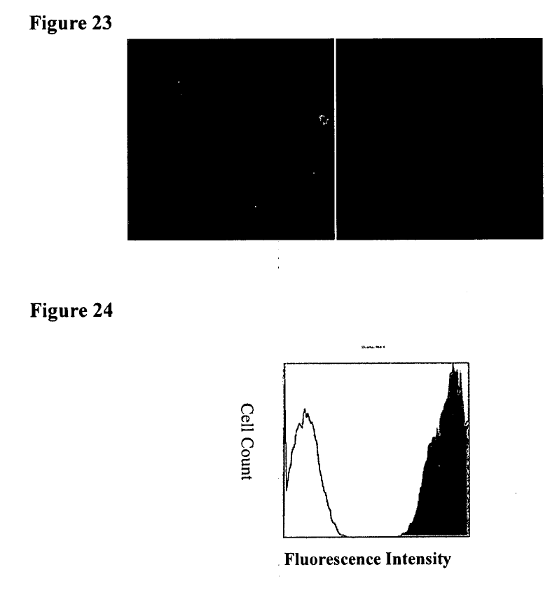

Internalization of PLGA nanoparticle in human DCs—Human imDCs were cultured in the complete RPMI 1640 medium containing GM-CSF and IL-4 for 7 days, and were then exposed to PLGA nanoparticles containing fluorescein (coumarin 6) and Mart-1:27-35 with a concentration of 100 μg/ml for 1 h. These human imDCs were harvested to test nanoparticle internalization by fluorescence microscopy (FIG. 23). In addition, human imDCs were incubated with nanoparticles containing coumarin 6 and Mart-1:27-35 peptide, and analyzed by FACS. The results showed that 100% of human DCs phagocytosed the nanoparticles after the incubation (FIG. 24).

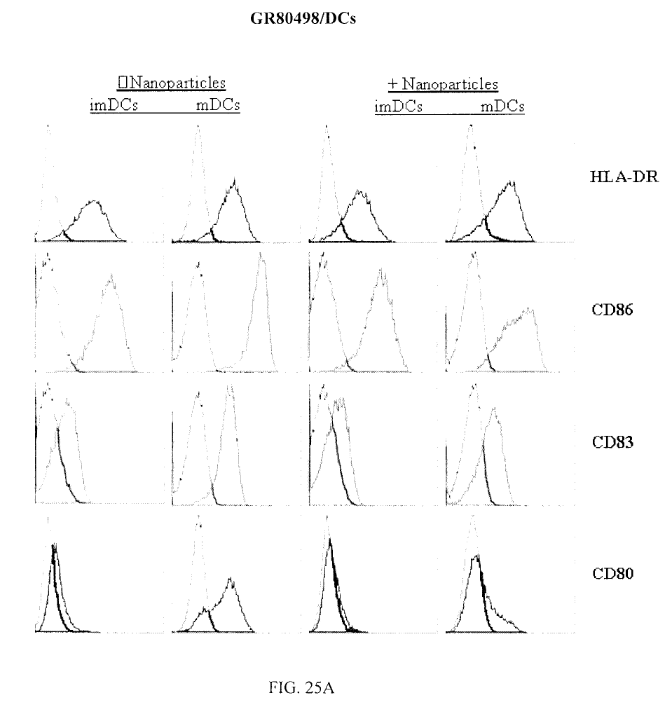

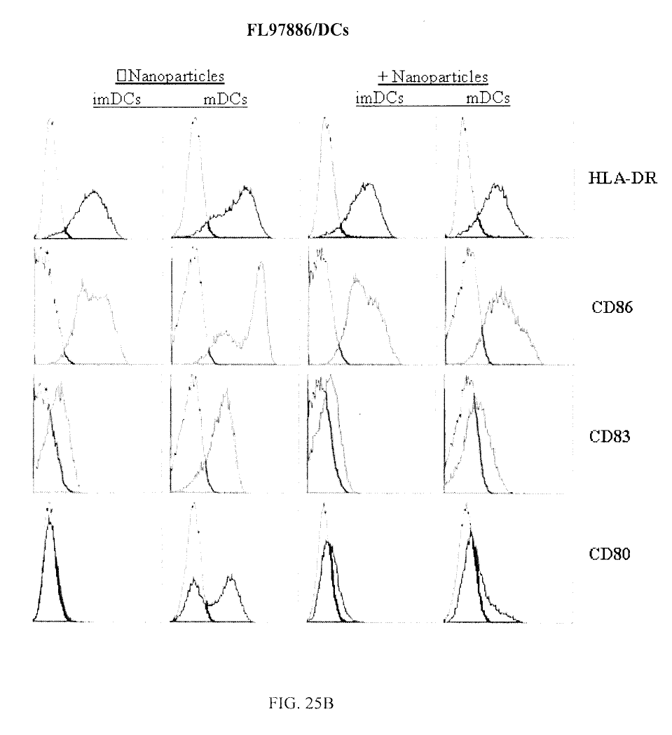

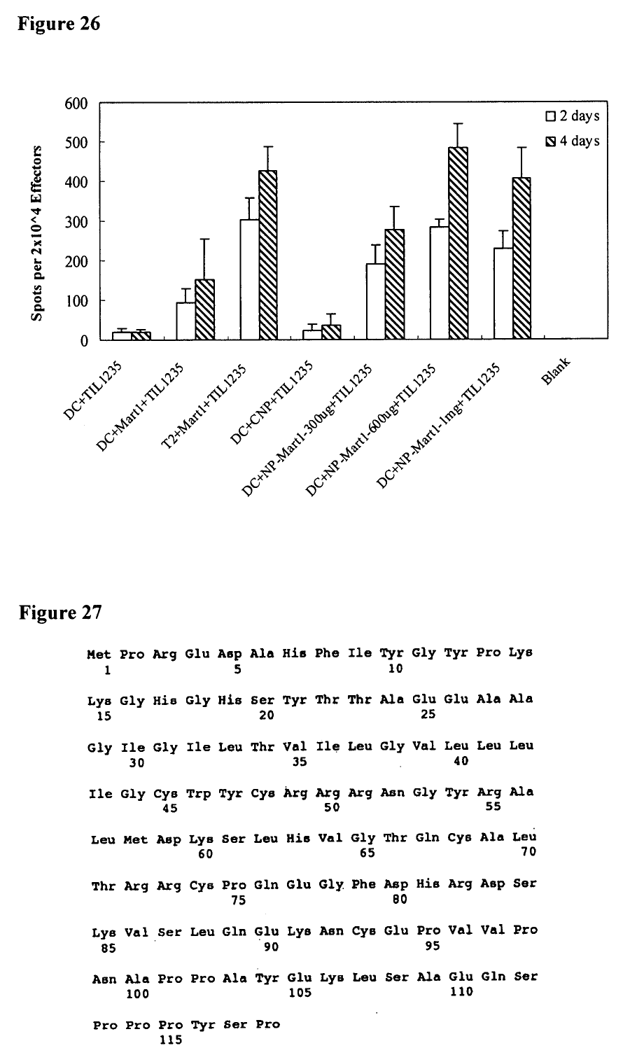

Effect of PLGA nanoparticle uptake on the character of the human DCs—This experiment sought to determine whether an incubation of human imDCs with PLGA nanoparticles would cause the maturation of DCs. The tested DC surface markers CD80, CD83, CD86 and HLA-DR were slightly increased after incubation with the PLGA nanoparticles. A similar study found that a maturation process was induced by the nanospheres as the maturation markers HLA-DR and CD86 were upregulated (Matsusaki, 2005 #34). Also, a similar type of biodegradable nanoparticles had some effect on the maturation of Human Cord Blood Derived Dendritic Cells (Diwan, 2003 #37). The FACS analysis was performed on a FACScan and was analyzed using the Cell Quest software. Human DCs were stained with the antibodies for 30 min at 4° C., washed, and resuspended in PBS, containing 0.5% BSA. Gates were set to exclude debris and nonviable cells. The following antibodies were used for FACS analysis: PE-anti-human HLA-DR, PE-anti-human CD83, FITC-anti-human CD80, FITC-anti-human CD86, FITC-mouse IgG1, PE-mouse IgG1, and PE-mouse IgG2a. FITC-mouse IgG1, PE mouse IgG1, and PE mouse IgG2a were used to determine the level of background staining (FIGS. 25A and 25B).

Prolonged and enhanced antigen presentation of human DCs containing nanoparticles-Mart-1:27-35 peptide determined by ELISpot—PLGA nanoparticles have the unique feature of slowly releasing antigen in a continuous, sustained fashion (Moynihan, 2001 #26). The ELISpot assay measured the IFN-γ release from the tumor infiltrating lymphocytes TIL1235, in response to Mart-1:27-35 peptide (AAGIGILTV) (SEQ ID NO: 194) being expressed by human DCs. On day 5 of the DC cultures, the DCs were incubated with the PLGA nanoparticles (100 μg/ml) containing Mart-1:27-35. To induce maturation, LPS was added at a concentration of 100 ng/ml on day 7. Also on day 7, the control DCs were incubated with the same amount of NPs containing Mart 1:27-35 peptide for 1 h, and the same amount of LPS was added. All DCs were then incubated for 2 days. All the DCs were collected on day 9 and the peptide presentation was tested by ELISpot assay. As a positive control, human DCs were incubated with the peptide Mart-1:27-35 on day 9, and compared with the DCs incubated with NPs containing the same peptide. T2 cells pulsed with Mart-1:27-35 was used as a general positive control. Human DCs containing nanoparticles without any peptide (CNP) and non-loaded DCs were included as negative controls. The spots in the wells representing DCs tested two days and four days after NP-loading were compared using t test. The p values were less than 0.005 (0.002613755, 5.75637E-07 and 7.98083E-06 for 300 μg batch, 600 μg batch and 1 mg batch respectively), which shows a significant difference between these groups. This finding also suggested that there was a controlled release of Mart-1:27-35 peptide inside the DCs for a prolonged antigen presentation. These experiments demonstrated that human DCs that phagocytosed nanoparticles containing Mart-1:27-35 peptide could present this peptide much more efficiently than the soluble Mart-1 peptide.

The nanoparticle batches prepared with different amounts of the Mart-1:27-35 peptide were also compared. The statistical analysis showed that 600 μg of Mart-1:27-35 peptide encapsulated in 30 mg of PLGA nanoparticles produced the most efficient nanoparticles. The p values were less than 0.005 (0.00424, 0.00000025, and 0.00000414 respectively) when the DCs loaded with NPs containing 300 μg, 600 μg and 1 mg of the peptide were compared with the DCs loaded directly with the soluble peptide on day 9. The difference in the peptide presentation between the DCs loaded with NP-Mart-1 for 4 days and 2 days was very significant. It indicated that the Mart-1:27-35 peptide inside human DCs was effectively presented to the responders (TIL1235) for at least four days.

Although the invention has been described with reference to the above examples, it will be understood that modifications and variations are encompassed within the spirit and scope of the invention. Accordingly, the invention is limited only by the following claims.

What is claimed is:

1. A nanoparticle containing an isolated class I restricted peptide, wherein the peptide is selected from the group consisting of SEQ ID NOs: 2 to 67, 71 to 94, 96 to 159 and 194.

2. The nanoparticle of claim 1, wherein the peptide is modified by N-terminal acetylation or C-terminal amidation.

3. The nanoparticle of claim 1, wherein a signal sequence is operably linked to the peptide.

4. The nanoparticle of claim 1, wherein the nanoparticle is formulated from Poly(D,L-lactide-co-glycolide) (PLGA).

5. A method of treating or preventing cancer in a subject comprising administering to a subject in need thereof a nanoparticle of claim 1, thereby treating or preventing cancer in the subject.

6. The method of claim 5, wherein the cancer is any cancer cell expressing the antigens PRAME, OFA/iLP, STEAP, SURVIVIN, or MART-1.

7. The method of claim 6, wherein the cancer is selected from the group consisting of lung cancer, breast cancer, prostate cancer, and a brain tumor.

8. The method of claim 5, further comprising administering a therapeutic agent in combination with the nanoparticle.

9. The method of claim 8, wherein the therapeutic agent is an anticancer agent or an antiviral agent.

10. A nanoparticle containing a fusion peptide, wherein the fusion peptide comprises a signal sequence and an antigen-derived peptide from an antigen expressed on the surface of a cancer cell or a virus-infected cell.

11. The nanoparticle of claim 10, wherein the antigen-derived peptide is selected from any one of SEQ ID NOs: 132-159.

12. The nanoparticle of claim 10, wherein the antigen-derived peptide is Mart-1:27-35 (SEQ ID NO: 194).

13. The nanoparticle of claim 10, wherein the cell is a cancer cell selected from the group consisting of a cancerous prostate cell, cancerous breast cell, cancerous lung cell, and a brain tumor cell.

14. A method of treating or preventing cancer in a subject comprising administering to a subject in need thereof of a nanoparticle of claim 18, thereby treating or preventing cancer in the subject.

15. The method of claim 14, wherein the cancer is prostate cancer, breast cancer, lung cancer, or a brain tumor.

16. The method of claim 14, further comprising administering a therapeutic agent in combination with the nanoparticle.

17. The method of claim 16, wherein the therapeutic agent is an anticancer agent or an antiviral agent.

18. A method of treating or preventing a viral disease in a subject comprising administering to a subject in need there of a nanoparticle of claim 18, thereby treating or preventing a viral disease in the subject.

19. The method of claim 18, further comprising administering a therapeutic agent in combination with the nanoparticle.

20. The method of claim 19, wherein the therapeutic agent is an anticancer agent or an antiviral agent.


SEQUENCE LISTING (194 sequences)

SEQ ID NO: 1 - protein, 509 aa, Homo sapiens
MERRRLWGSIQSRYISMSVWTSPRRLVELAGQSLLKDEALAIAALELLPRELFPPLFMAAFDGRHSQTLKAMVQAWPFTCLPLGVLMKGQHLHLETFKAVLDGLDVLLAQEVRPRRWKLQVLDLRKNSHQDFWTVWSGNRASLYSFPEPEAAQPMTKKRKVDGLSTEAEQPFIPVEVLVDLFLKEGACDELFSYLIEKVKRKKNVLRLCCKKLKIFAMPMQDIKMILKMVQLDSIEDLEVTCTWKLPTLAKFSPYLGQMINLRRLLLSHIHASSYISPEKEEQYIAQFTSQFLSLQCLQALYVDSLFFLRGRLDQLLRHVMNPLETLSITNCRLSEGDVMHLSQSPSVSQLSVLSLSGVMLTDVSPEPLQALLERASATLQDLVFDECGITDDQLLALLPSLSHCSQLTTLSFYGNSISISALQSLLQHLIGLSNLTHVLYPVPLESYEDIHGTLHLERLAYLHARLRELLCELGRPSMVWLSANPCPHCGDRTFYDPEPILCPCFMPN

SEQ ID NO: 2 - protein, 9 aa, Homo sapiens
VLDGLDVLL

SEQ ID NO: 3 - protein, 10 aa, Homo sapiens
SLYSFPEPEA

SEQ ID NO: 4 - protein, 10 aa, Homo sapiens
ALYVDSLFFL

SEQ ID NO: 5 - protein, 9 aa, Homo sapiens
SLLQHLIGL

SEQ ID NO: 6 - protein, 9 aa, Artificial sequence
  Synthetic construct
  MOD_RES (1)..(1): ACETYLATION
VLDGLDVLL

SEQ ID NO: 7 - protein, 9 aa, Artificial sequence
  Synthetic construct
  MOD_RES (9)..(9): AMIDATION
VLDGLDVLL

SEQ ID NO: 8 - protein, 9 aa, Artificial sequence
  Synthetic construct
  MOD_RES (1)..(1): ACETYLATION
  MOD_RES (1)..(1): AMIDATION
VLDGLDVLL

SEQ ID NO: 9 - protein, 10 aa, Artificial sequence
  Synthetic construct
  MOD_RES (1)..(1): ACETYLATION
SLYSFPEPEA

SEQ ID NO: 10 - protein, 10 aa, Artificial sequence
  Synthetic construct
  MOD_RES (10)..(10): AMIDATION
SLYSFPEPEA

SEQ ID NO: 11 - protein, 10 aa, Artificial sequence
  Synthetic construct
  MOD_RES (1)..(1): ACETYLATION
  MOD_RES (10)..(10): AMIDATION
SLYSFPEPEA

SEQ ID NO: 12 - protein, 10 aa, Artificial sequence
  Synthetic construct
  MOD_RES (1)..(1): ACETYLATION
ALYVDSLFFL

SEQ ID NO: 13 - protein, 10 aa, Artificial sequence
  Synthetic construct
  MOD_RES (10)..(10): AMIDATION
ALYVDSLFFL

SEQ ID NO: 14 - protein, 10 aa, Artificial sequence
  Synthetic construct
  MOD_RES (1)..(1): ACETYLATION
  MOD_RES (10)..(10): AMIDATION
ALYVDSLFFL

SEQ ID NO: 15 - protein, 9 aa, Artificial sequence
  Synthetic construct
  MOD_RES (1)..(1): ACETYLATION
SLLQHLIGL

SEQ ID NO: 16 - protein, 9 aa, Artificial sequence
  Synthetic construct
  MOD_RES (9)..(9): AMIDATION
SLLQHLIGL

SEQ ID NO: 17 - protein, 9 aa, Artificial sequence
  Synthetic construct
  MOD_RES (1)..(1): ACETYLATION
  MOD_RES (9)..(9): AMIDATION
SLLQHLIGL

SEQ ID NO: 18 - protein, 9 aa, Artificial sequence
  Synthetic construct
FLDGLDVLL

SEQ ID NO: 19 - protein, 9 aa, Artificial sequence
  Synthetic construct
VLWGLDVLL

SEQ ID NO: 20 - protein, 9 aa, Artificial sequence
  Synthetic construct
VLDGLDVLV

SEQ ID NO: 21 - protein, 9 aa, Artificial sequence
  Synthetic construct
FLWGLDVLV

SEQ ID NO: 22 - protein, 10 aa, Artificial sequence
  Synthetic construct
FLYSFPEPEA

SEQ ID NO: 23 - protein, 10 aa, Artificial sequence
  Synthetic construct
SLWSFPEPEA

SEQ ID NO: 24 - protein, 10 aa, Artificial sequence
  Synthetic construct
SLYSFPEPEV

SEQ ID NO: 25 - protein, 10 aa, Artificial sequence
  Synthetic construct
FLWSFPEPEV

SEQ ID NO: 26 - protein, 10 aa, Artificial sequence
  Synthetic construct
ALFVDSLFFL

SEQ ID NO: 27 - protein, 10 aa, Artificial sequence
  Synthetic construct
ALYVDSLFFV

SEQ ID NO: 28 - protein, 10 aa, Artificial sequence
  Synthetic construct
ALFVDSLFFV

SEQ ID NO: 29 - protein, 9 aa, Artificial sequence
  Synthetic construct
FLLQHLIGL

SEQ ID NO: 30 - protein, 9 aa, Artificial sequence
  Synthetic construct
SLWQHLIGL

SEQ ID NO: 31 - protein, 9 aa, Artificial sequence
  Synthetic construct
SLLQHLIGV

SEQ ID NO: 32 - protein, 9 aa, Artificial sequence
  Synthetic construct
FLWQHIIGV

SEQ ID NO: 33 - protein, 9 aa, Artificial sequence
  Synthetic construct
VLKGLDVLL

SEQ ID NO: 34 - protein, 9 aa, Artificial sequence
  Synthetic construct
VLDGHDVLL

SEQ ID NO: 35 - protein, 9 aa, Artificial sequence
  Synthetic construct
VLDGLDPLL

SEQ ID NO: 36 - protein, 10 aa, Artificial sequence
  Synthetic construct
SLKSFPEPEA

SEQ ID NO: 37 - protein, 10 aa, Artificial sequence
  Synthetic construct
SLYSHPEPEA

SEQ ID NO: 38 - protein, 10 aa, Artificial sequence
  Synthetic construct
SLYSFPPPEA

SEQ ID NO: 39 - protein, 10 aa, Artificial sequence
  Synthetic construct
ALKVDSLFFL

SEQ ID NO: 40 - protein, 10 aa, Artificial sequence
  Synthetic construct
ALYVHSLFFL

SEQ ID NO: 41 - protein, 10 aa, Artificial sequence
  Synthetic construct
ALYVDSPFFL

SEQ ID NO: 42 - protein, 9 aa, Artificial sequence
  Synthetic construct
SLKQHLIGL

SEQ ID NO: 43 - protein, 9 aa, Artificial sequence
  Synthetic construct
SLLQHLPGL

SEQ ID NO: 44 - protein, 26 aa, Artificial sequence
  Synthetic construct
MRYMILGLLALAAVCSAVLDGLDVLL

SEQ ID NO: 45 - protein, 26 aa, Artificial sequence
  Synthetic construct
VLDGLDVLLMRYMILGLLALAAVCSA

SEQ ID NO: 46 - protein, 30 aa, Artificial sequence
  Synthetic construct
MTNKCLLQIALLLCFSTTALSVLDGLDVLL

SEQ ID NO: 47 - protein, 30 aa, Artificial sequence
  Synthetic construct
VLDGLDVLLMTNKCLLQIALLLCFSTTALS

SEQ ID NO: 48 - protein, 17 aa, Artificial sequence
  Synthetic construct
MRVLDGLDVLLAAVCSA

SEQ ID NO: 49 - protein, 17 aa, Artificial sequence
  Synthetic construct
MAVLDGLDVLLAAAAAG

SEQ ID NO: 50 - protein, 27 aa, Artificial sequence
  Synthetic construct
MRYMILGLLALAAVCSASLYSFPEPEA

SEQ ID NO: 51 - protein, 27 aa, Artificial sequence
  Synthetic construct
SLYSFPEPEAMRYMILGLLALAAVCSA

SEQ ID NO: 52 - protein, 31 aa, Artificial sequence
  Synthetic construct
MTNKCLLQIALLLCFSTTALSSLYSFPEPEA

SEQ ID NO: 53 - protein, 31 aa, Artificial sequence
  Synthetic construct
SLYSFPEPEAMTNKCLLQIALLLCFSTTALS

SEQ ID NO: 54 - protein, 18 aa, Artificial sequence
  Synthetic construct
MRSLYSFPEPEAAAVCSA

SEQ ID NO: 55 - protein, 18 aa, Artificial sequence
  Synthetic construct
MASLYSFPEPEAAAAAAG

SEQ ID NO: 56 - protein, 27 aa, Artificial sequence
  Synthetic construct
MRYMILGLLALAAVCSAALYVDSLFFL

SEQ ID NO: 57 - protein, 27 aa, Artificial sequence
  Synthetic construct
ALYVDSLFFLMRYMILGLLALAAVCSA

SEQ ID NO: 58 - protein, 31 aa, Artificial sequence
  Synthetic construct
MTNKCLLQIALLLCFSTTALSALYVDSLFFL

SEQ ID NO: 59 - protein, 31 aa, Artificial sequence
  Synthetic construct
ALYVDSLFFLMTNKCLLQIALLLCFSTTALS

SEQ ID NO: 60 - protein, 18 aa, Artificial sequence
  Synthetic construct
MRALYVDSLFFLAAVCSA

SEQ ID NO: 61 - protein, 18 aa, Artificial sequence
  Synthetic construct
MAALYVDSLFFLAAAAAG

SEQ ID NO: 62 - protein, 26 aa, Artificial sequence
  Synthetic construct
MRYMILGLLALAAVCSASLLQHLIGL

SEQ ID NO: 63 - protein, 26 aa, Artificial sequence
  Synthetic construct
SLLQHLIGLMRYMILGLLALAAVCSA

SEQ ID NO: 64 - protein, 30 aa, Artificial sequence
  Synthetic construct
MTNKCLLQIALLLCFSTTALSSLLQHLIGL

SEQ ID NO: 65 - protein, 30 aa, Artificial sequence
  Synthetic construct
SLLQHLIGLMTNKCLLQIALLLCFSTTALS

SEQ ID NO: 66 - protein, 17 aa, Artificial sequence
  Synthetic construct
MRSLLQHLIGLAAVCSA

SEQ ID NO: 67 - protein, 17 aa, Artificial sequence
  Synthetic construct
MASLLQHLIGLAAAAAG

SEQ ID NO: 68 - protein, 17 aa, Artificial sequence
  Synthetic construct
MRYMILGLLALAAVCSA

SEQ ID NO: 69 - protein, 21 aa, Artificial sequence
  Synthetic construct
MTNKCLLQIALLLCFSTTALS

SEQ ID NO: 70 - protein, 295 aa, Homo sapiens
MSGALDVLQMKEEDVLKFLAAGTHLGGTNLDFQMEQYIYKRKSDGIYIINLKRTWEKLLLAARAIVAIENPADVSVISSRNTGQRAVLKFAAATGATPIAGRFTPGTFTNQIQAAFREPRLLVVTDPRADHQPLTEASYVNLPTIALCNTDSPLRYVDIAIPCNNKGAHSVGLMWWMLAREVLRMRGTISREHPWEVMPDLYFYRDPEEIEKEEQAAAEKAVTKEEFQGEWTAPAPEFTAAQPEVADWSEGVQVPSVPIQQFPTEDWSAQPATEDWSAAPTAQATEWVGATTEWS

SEQ ID NO: 71 - protein, 9 aa, Homo sapiens
VLQMKEEDV

SEQ ID NO: 72 - protein, 9 aa, Homo sapiens
NLKRTWEKL

SEQ ID NO: 73 - protein, 9 aa, Homo sapiens
KLLLAARAI

SEQ ID NO: 74 - protein, 9 aa, Homo sapiens
LLLAARAIV

SEQ ID NO: 75 - protein, 9 aa, Homo sapiens
ALCNTDSPL

SEQ ID NO: 76 - protein, 9 aa, Homo sapiens
PLRYVDIAI

SEQ ID NO: 77 - protein, 9 aa, Artificial sequence
  Synthetic construct
  MOD_RES (1)..(1): ACETYLATION
LLLAARAIV

SEQ ID NO: 78 - DNA, 1057 bp, Homo sapiens
gtcgacccacgcgtccgctacccggggacgggtccatacggcgttgttcttgattcccatcgtaacttaaagggaaacttacacaatgtccggagcccttgacgtcctgcagatgaaggaggaggatgtcctcaaattccttgctgcgggaacccacttaggtggcaccaaccttgactttcagatggagcagtacatctacaaaaggaaaagtgacggtatctacatcataaacctgaagaggacctgggagaagctgttgctcgcagctcgagctattgttgccatcgagaatcctgctgacgtcagcgtcatctcctccaggaacactggccagcgagctgtgctgaagtttgctgctgccacaggagccactccgatcgctggccgcttcacacctgggaccttcactaaccagatccaagcagccttcagggagccacggcttctagtggtgaccgatcccagggctgaccatcagccactcacagaggcctcttatgtcaacctgcccaccattgctctgtgtaacacagattctcccctgcgctatgtggacattgccatcccatgcaacaacaagggagctcactcagtgggtctgatgtggtggatgctggccagggaagtactccgcatgcgaggtactatctcccgtgagcacccctgggaggtcatgcctgatctttacttctacagagacccagaggagattgagaaggaggagcaggctgctgctgagaaggctgtgaccaaggaggaattccagggtgaatggaccgcaccagctcctgagttcactgctgctcagcctgaggtggccgactggtctgagggtgtgcaggttccctctgtgcccatccagcagttccccacggaagactggagtgcacagccagccactgaggattggtcagcagctcccacagcgcaggccactgagtgggttggagccaccactgagtggtcctgagctgctgtgcaggtgcctgagcaaagggaaaaaagatggaaggaaaataaagttgctaaaagctgaaaaaaaaaaaaaaaaaaa

SEQ ID NO: 79 - protein, 9 aa, Artificial sequence
  Synthetic construct
  MOD_RES (9)..(9): AMIDATION
LLLAARAIV

SEQ ID NO: 80 - protein, 9 aa, Artificial sequence
  Synthetic construct
  MOD_RES (1)..(1): ACETYLATION
  MOD_RES (9)..(9): AMIDATION
LLLAARAIV

SEQ ID NO: 81 - protein, 9 aa, Artificial sequence
  Synthetic construct
FLLLAARAI

SEQ ID NO: 82 - protein, 9 aa, Artificial sequence
  Synthetic construct
KLWLAARAI

SEQ ID NO: 83 - protein, 9 aa, Artificial sequence
  Synthetic construct
KLLLAARAV

SEQ ID NO: 84 - protein, 9 aa, Artificial sequence
  Synthetic construct
FLWLAARAV

SEQ ID NO: 85 - protein, 9 aa, Artificial sequence
  Synthetic construct
VLKMKEEDV

SEQ ID NO: 86 - protein, 9 aa, Artificial sequence
  Synthetic construct
VLQMHEEDV

SEQ ID NO: 87 - protein, 9 aa, Artificial sequence
  Synthetic construct
VLQMKEPDV

SEQ ID NO: 88 - protein, 118 aa, Homo sapiens
MPREDAHFIYGYPKKGHGHSYTTAEEAAGIGILTVILGVLLLIGCWYCRRRNGYRALMDKSLHVGTQCALTRRCPQEGFDHRDSKVSLQEKNCEPVVPNAPPAYEKLSAEQSPPPYSP

SEQ ID NO: 89 - protein, 26 aa, Artificial sequence
  Synthetic construct
MRYMILGLLALAAVCSALLLAARAIV

SEQ ID NO: 90 - protein, 26 aa, Artificial sequence
  Synthetic construct
LLLAARAIVMRYMILGLLALAAVCSA

SEQ ID NO: 91 - protein, 30 aa, Artificial sequence
  Synthetic construct
MTNKCLLQIALLLCFSTTALSLLLAARAIV

SEQ ID NO: 92 - protein, 30 aa, Artificial sequence
  Synthetic construct
LLLAARAIVMTNKCLLQIALLLCFSTTALS

SEQ ID NO: 93 - protein, 17 aa, Artificial sequence
  Synthetic construct
MRLLLAARAIVAAVCSA

SEQ ID NO: 94 - protein, 17 aa, Artificial sequence
  Synthetic construct
MALLLAARAIVAAAAAG

SEQ ID NO: 95 - protein, 339 aa, Homo sapiens
MESRKDITNQEELWKMKPRRNLEEDDYLHKDTGETSMLKRPVLLHLHQTAHADEFDCPSELQHTQELFPQWHLPIKIAAIIASLTFLYTLLREVIHPLATSHQQYFYKIPILVINKVLPMVSITLLALVYLPGVIAAIVQLHNGTKYKKFPHWLDKWMLTRKQFGLLSFFFAVLHAIYSLSYPMRRSYRYKLLNWAYQQVQQNKEDAWIEHDVWRMEIYVSLGIVGLAILALLAVTSIPSVSDSLTWREFHYIQSKLGIVSLLLGTIHALIFAWNKWIDIKQFVWYTPPTFMIAVFLPIVVLIFKSILFLPCLRKKILKIRHGWEDVTKINKTEICSQL

SEQ ID NO: 96 - protein, 9 aa, Homo sapiens
FLYTLLREV

SEQ ID NO: 97 - protein, 9 aa, Homo sapiens
SLTFLYTLL

SEQ ID NO: 98 - protein, 9 aa, Homo sapiens
HLPIKIAAI

SEQ ID NO: 99 - protein, 9 aa, Homo sapiens
SMLKRPVLL

SEQ ID NO: 100 - protein, 9 aa, Homo sapiens
GLLSFFFAV

SEQ ID NO: 101 - protein, 9 aa, Homo sapiens
LLREVIHPL

SEQ ID NO: 102 - protein, 9 aa, Homo sapiens
ALVYLPGVI

SEQ ID NO: 103 - protein, 9 aa, Homo sapiens
FMIAVFLPI

SEQ ID NO: 104 - protein, 9 aa, Homo sapiens
LLNWAYQQV

SEQ ID NO: 105 - protein, 9 aa, Homo sapiens
VLPMVSITL

SEQ ID NO: 106 - protein, 9 aa, Homo sapiens
YLPGVIAAI

SEQ ID NO: 107 - protein, 9 aa, Homo sapiens
MLTRKQFGL

SEQ ID NO: 108 - protein, 9 aa, Homo sapiens
SLGIVGLAI

SEQ ID NO: 109 - protein, 9 aa, Homo sapiens
LLSFFFAVL

SEQ ID NO: 110 - protein, 9 aa, Homo sapiens
LLGTIHALI

SEQ ID NO: 111 - protein, 9 aa, Homo sapiens
KLGIVSLLL

SEQ ID NO: 112 - protein, 9 aa, Homo sapiens
FLPCLRKKI

SEQ ID NO: 113 - protein, 9 aa, Homo sapiens
LLLGTIHAL

SEQ ID NO: 114 - protein, 9 aa, Homo sapiens
CLRKKILKI

SEQ ID NO: 115 - protein, 9 aa, Homo sapiens
LIFKSILFL

SEQ ID NO: 116 - protein, 9 aa, Artificial sequence
  Synthetic construct
  MOD_RES (1)..(1): ACETYLATION
YLPGVIAAI

SEQ ID NO: 117 - protein, 9 aa, Artificial sequence
  Synthetic construct
  MOD_RES (9)..(9): AMIDATION
YLPGVIAAI

SEQ ID NO: 118 - protein, 9 aa, Artificial sequence
  Synthetic construct
  MOD_RES (1)..(1): ACETYLATION
  MOD_RES (9)..(9): AMIDATION
YLPGVIAAI

SEQ ID NO: 119 - protein, 9 aa, Artificial sequence
  Synthetic construct
FLPGVIAAI

SEQ ID NO: 120 - protein, 9 aa, Artificial sequence
  Synthetic construct
YLWGVIAAI

SEQ ID NO: 121 - protein, 9 aa, Artificial sequence
  Synthetic construct
YLPGVIAAV

SEQ ID NO: 122 - protein, 9 aa, Artificial sequence
  Synthetic construct
FLWGVIAAV

SEQ ID NO: 123 - protein, 9 aa, Artificial sequence
  Synthetic construct
YLKGVIAAI

SEQ ID NO: 124 - protein, 9 aa, Artificial sequence
  Synthetic construct
YLPGHIAAI

SEQ ID NO: 125 - protein, 9 aa, Artificial sequence
  Synthetic construct
YLPGVIPAI

SEQ ID NO: 126 - protein, 26 aa, Artificial sequence
  Synthetic construct
MRYMILGLLALAAVCSAYLPGVIAAI

SEQ ID NO: 127 - protein, 26 aa, Artificial sequence
  Synthetic construct
YLPGVIAAIMRYMILGLLALAAVCSA

SEQ ID NO: 128 - protein, 30 aa, Artificial sequence
  Synthetic construct
MTNKCLLQIALLLCFSTTALSYLPGVIAAI

SEQ ID NO: 129 - protein, 30 aa, Artificial sequence
  Synthetic construct
YLPGVIAAIMTNKCLLQIALLLCFSTTALS

SEQ ID NO: 130 - protein, 17 aa, Artificial sequence
  Synthetic construct
MRYLPGVIAAIAAVCSA

SEQ ID NO: 131 - protein, 17 aa, Artificial sequence
  Synthetic construct
MAYLPGVIAAIAAAAAG

SEQ ID NO: 132 - protein, 9 aa, Homo sapiens
HLYQGCQVV

SEQ ID NO: 133 - protein, 9 aa, Homo sapiens
KIFGSLAFL

SEQ ID NO: 134 - protein, 9 aa, Homo sapiens
IISAVVGIL

SEQ ID NO: 135 - protein, 9 aa, Homo sapiens
CLTSTVQLV

SEQ ID NO: 136 - protein, 26 aa, Artificial sequence
  Synthetic construct
MRYMILGLLALAAVCSAHLYQGCQVV

SEQ ID NO: 137 - protein, 26 aa, Artificial sequence
  Synthetic construct
HLYQGCQVVMRYMILGLLALAAVCSA

SEQ ID NO: 138 - protein, 30 aa, Artificial sequence
  Synthetic construct
MTNKCLLQIALLLCFSTTALSHLYQGCQVV

SEQ ID NO: 139 - protein, 30 aa, Artificial sequence
  Synthetic construct
HLYQGCQVVMTNKCLLQIALLLCFSTTALS

SEQ ID NO: 140 - protein, 17 aa, Artificial sequence
  Synthetic construct
MRHLYQGCQVVAAVCSA

SEQ ID NO: 141 - protein, 17 aa, Artificial sequence
  Synthetic construct
MAHLYQGCQVVAAAAAG

SEQ ID NO: 142 - protein, 26 aa, Artificial sequence
  Synthetic construct
MRYMILGLLALAAVCSAKIFGSLAFL

SEQ ID NO: 143 - protein, 26 aa, Artificial sequence
  Synthetic construct
KIFGSLAFLMRYMILGLLALAAVCSA

SEQ ID NO: 144 - protein, 30 aa, Artificial sequence
  Synthetic construct
MTNKCLLQIALLLCFSTTALSKIFGSLAFL

SEQ ID NO: 145 - protein, 30 aa, Artificial sequence
  Synthetic construct
KIFGSLAFLMTNKCLLQIALLLCFSTTALS

SEQ ID NO: 146 - protein, 17 aa, Artificial sequence
  Synthetic construct
MRKIFGSLAFLAAVCSA

SEQ ID NO: 147 - protein, 17 aa, Artificial sequence
  Synthetic construct
MAKIFGSLAFLAAAAAG

SEQ ID NO: 148 - protein, 26 aa, Artificial sequence
  Synthetic construct
MRYMILGLLALAAVCSAIISAVVGIL

SEQ ID NO: 149 - protein, 26 aa, Artificial sequence
  Synthetic construct
IISAVVGILMRYMILGLLALAAVCSA

SEQ ID NO: 150 - protein, 30 aa, Artificial sequence
  Synthetic construct
MTNKCLLQIALLLCFSTTALSIISAVVGIL

SEQ ID NO: 151 - protein, 30 aa, Artificial sequence
  Synthetic construct
IISAVVGILMTNKCLLQIALLLCFSTTALS

SEQ ID NO: 152 - protein, 17 aa, Artificial sequence
  Synthetic construct
MRIISAVVGILAAVCSA

SEQ ID NO: 153 - protein, 17 aa, Artificial sequence
  Synthetic construct
MAIISAVVGILAAAAAG

SEQ ID NO: 154 - protein, 26 aa, Artificial sequence
  Synthetic construct
MRYMILGLLALAAVCSACLTSTVQLV

SEQ ID NO: 155 - protein, 26 aa, Artificial sequence
  Synthetic construct
CLTSTVQLVMRYMILGLLALAAVCSA

SEQ ID NO: 156 - protein, 30 aa, Artificial sequence
  Synthetic construct
MTNKCLLQIALLLCFSTTALSCLTSTVQLV

SEQ ID NO: 157 - protein, 28 aa, Artificial sequence
  Synthetic construct
CLTSTVQLVMTNKCLLQIALLLCFSTTA

SEQ ID NO: 158 - protein, 17 aa, Artificial sequence
  Synthetic construct
MRCLTSTVQLVAAVCSA

SEQ ID NO: 159 - protein, 17 aa, Artificial sequence
  Synthetic construct
MACLTSTVQLVAAAAAG

SEQ ID NO: 160 - protein, 9 aa, Homo sapiens
STFKNWPFL

SEQ ID NO: 161 - protein, 10 aa, Homo sapiens
TLPPAWQPFL

SEQ ID NO: 162 - protein, 9 aa, Homo sapiens
KNWPFLEGC

SEQ ID NO: 163 - protein, 10 aa, Homo sapiens
KEFEETAKKV

SEQ ID NO: 164 - protein, 9 aa, Homo sapiens
LTLGEFLKL

SEQ ID NO: 165 - protein, 10 aa, Homo sapiens
ELTLGEFLKL

SEQ ID NO: 166 - protein, 9 aa, Homo sapiens
LPPAWQPFL

SEQ ID NO: 167 - protein, 10 aa, Homo sapiens
ISTFKNWPFL

SEQ ID NO: 168 - protein, 9 aa, Homo sapiens
CTPERMAEA

SEQ ID NO: 169 - protein, 10 aa, Homo sapiens
TENEPDLAQC

SEQ ID NO: 170 - protein, 9 aa, Homo sapiens
CPTENEPDL

SEQ ID NO: 171 - protein, 10 aa, Homo sapiens
FEELTLGEFL

SEQ ID NO: 172 - protein, 9 aa, Homo sapiens
KVRRAIEQL

SEQ ID NO: 173 - protein, 10 aa, Homo sapiens
LSVKKQFEEL

SEQ ID NO: 174 - protein, 9 aa, Homo sapiens
RMAEAGFIH

SEQ ID NO: 175 - protein, 10 aa, Homo sapiens
KKVRRAIEQL

SEQ ID NO: 176 - protein, 9 aa, Homo sapiens
SVKKQFEEL

SEQ ID NO: 177 - protein, 10 aa, Homo sapiens
FLKDHRISTF

SEQ ID NO: 178 - protein, 10 aa, Homo sapiens
ACTPERMAEA

SEQ ID NO: 179 - protein, 9 aa, Artificial sequence
  Synthetic construct
  MOD_RES (1)..(1): ACETYLATION
STFKNWPFL

SEQ ID NO: 180 - protein, 9 aa, Artificial sequence
  Synthetic construct
  MOD_RES (9)..(9): AMIDATION
STFKNWPFL

SEQ ID NO: 181 - protein, 9 aa, Artificial sequence
  Synthetic construct
  MOD_RES (1)..(1): ACETYLATION
  MOD_RES (9)..(9): AMIDATION
STFKNWPFL

SEQ ID NO: 182 - protein, 9 aa, Artificial sequence
  Synthetic construct
SLFKNWPFL

SEQ ID NO: 183 - protein, 9 aa, Artificial sequence
  Synthetic construct
ATFKNWPFL

SEQ ID NO: 184 - protein, 9 aa, Artificial sequence
  Synthetic construct
ALFKNWPFL

SEQ ID NO: 185 - protein, 9 aa, Artificial sequence
  Synthetic construct
STKKNWPFL

SEQ ID NO: 186 - protein, 9 aa, Artificial sequence
  Synthetic construct
STFKHWPFL

SEQ ID NO: 187 - protein, 26 aa, Artificial sequence
  Synthetic construct
MRYMILGLLALAAVCSASTFKNWPFL

SEQ ID NO: 188 - protein, 26 aa, Artificial sequence
  Synthetic construct
STFKNWPFLMRYMILGLLALAAVCSA

SEQ ID NO: 189 - protein, 30 aa, Artificial sequence
  Synthetic construct
MTNKCLLQIALLLCFSTTALSSTFKNWPFL

SEQ ID NO: 190 - protein, 30 aa, Artificial sequence
  Synthetic construct
STFKNWPFLMTNKCLLQIALLLCFSTTALS

SEQ ID NO: 191 - protein, 17 aa, Artificial sequence
  Synthetic construct
MRSTFKNWPFLAAVCSA

SEQ ID NO: 192 - protein, 17 aa, Artificial sequence
  Synthetic construct
MASTFKNWPFLAAAAAG

SEQ ID NO: 193 - protein, 142 aa, Homo sapiens
MGAPTLPPAWQPFLKDHRISTFKNWPFLEGCACTPERMAEAGFIHCPTENEPDLAQCFFCFKELEGWEPDDDPIEEHKKHSSGCAFLSVKKQFEELTLGEFLKLDRERAKNKIAKETNNKKKEFEETAKKVRRAIEQLAAMD

SEQ ID NO: 194 - protein, 9 aa, Artificial sequence
  Synthetic construct
AAGIGILTV